(19) United States
(12) Patent Application Publication
(10) Pub. No.: US 2024/0360143 A1
(43) Pub. Date: Oct. 31, 2024

(54) [1,2,4]TRIAZOLO[4,3-A]PYRIMIDIN-7(8H)-ONE AS MITOCHONDRIAL PYRUVATE CARRIER INHIBITORS FOR USE IN THE TREATMENT OF CANCER

(71) Applicants: MPC THERAPEUTICS SA, GENÈVE (CH); UNIVERSITE DE GENEVE, GENEVA 4 (CH)

(72) Inventors: BENJAMIN PERRY, PREVESSIN MOENS (FR); JEAN-CLAUDE MARTINOU, VERSONNEX (FR)

(21) Appl. No.: 18/682,951

(22) PCT Filed: Aug. 12, 2022
(86) PCT No.: PCT/EP2022/072681

§ 371 Date: Feb. 12, 2024

(30) Foreign Application Priority Data
Aug. 13, 2021 (EP) 21191387.6

(51) Int. Cl. C07D 487/04; A61K 31/519; A61K 39/00; C12N 5/0783

(52) U.S. Cl. CPC C07D 487/04; A61K 31/519; A61K 39/4611; A61K 39/4622; A61K 39/4631; A61K 39/4644; C12N 5/0636

(57) ABSTRACT

The present invention is related to compounds, methods, compositions and uses that are able to inhibit mitochondrial pyruvate carrier (MPC) activity and which are useful for immunotherapy, in particular T-cell therapies, immune check point inhibitors or anti-cancer vaccine.

### FIELD OF THE INVENTION

The present invention relates to the field of immunotherapy, in particular adoptive cell transfer immunotherapy and vaccinotherapy. In particular, the invention relates to mitochondrial pyruvate carrier (MPC) inhibitors and the use thereof.

### BACKGROUND OF THE INVENTION

Adoptive Cell Transfer (ACT) immunotherapy is the transfer of T-cells into a patient to treat a disease such as cancer or viral infection. Chimeric Antigen Receptor (CAR) T-cell is a type of ACT which involves the transfer T cells (either a patient's own or a donor's) which are genetically engineered ex-vivo to express a chimeric antigen receptor targeting a specific tumor antigen. CAR T therapy is emerging as one of the most promising approaches for relapse and refractory hematological malignancies. Indeed, it has already helped extremely ill patients beat diffuse large B-cell lymphoma (DLBCL) and acute lymphoblastic leukemia (ALL) where conventional treatments such as chemotherapy have failed (Wang et al., 2017, Journal of Hematology and Oncology; 10 (1): 53). However, CAR T therapy has only shown a limited effect on solid tumors, which account for the vast majority of cancer cases (approximately 90% of cancer cases in the US). In particular, it has been shown that the lack of proliferation and persistence of T-cells after infusion into the patient is responsible to a great extent for the poor effect of CAR-T therapy in solid tumors. Moreover, even for hematological malignancies with complete remission rate as high as to 90%, the patients remain at the risk of relapse because of the poor persistence of CAR-T cells in vivo (Gauthier et al., 2017, Curr Res Transl Med, 65 (3): 93-102). Accordingly, there is an unmet need to develop CAR T cells that survive, expand and persist in vivo.

One strategy is to infuse T cells with an early memory phenotype. According to their differentiation phase, T cells display a unique phenotype with associated functionalities and properties. Recently, it is becoming clear that clinical response is linked to T cell differentiation status (Lécuroux et al., 2009, Blood, 113 (14): 3209 3217). Historically, ACTs were using terminally differentiated T “effector” cells due to their potent killing capacity. However, T “effector” cells have a poor ability to expand and persist in vivo. It has been shown in mouse tumor models that the infusion of T cells with a self-renewing, early memory phenotype confers a stronger and more sustained anti-tumor response (Klebanoff et al., 2011, Clin Cancer Res., 17, 5343-5352). Furthermore, it has been shown that the inhibition or stimulation of certain metabolic processes alone is sufficient to induce effector versus memory differentiation, with corresponding in vivo effects when the cells are re-infused in infected or tumor-bearing mice (Balmer et al., 2016, Immunity, 44, 1312-1324; Phan et al., 2016, Immunity 45, 1024-1037). However, current strategies dramatically inhibit T cell proliferation and therefore are not suitable for use as a supplement during the ex vivo manufacturing process of T cells for ACT immunotherapy. Thus, there is a need for improved methods to obtain T-cells with a memory phenotype.

Further, there is a need to improve adjuvants for vaccines. Indeed, one of the main goals of preventive and therapeutic vaccination in the context of pathogen infection and cancer is the establishment of a potent memory T cell pool. Current adjuvants for vaccination are either aluminum salts and emulsions, or more specific pattern-recognition receptor agonists, all designed to target and activate antigen-presenting cells (APC). This does not always result in an efficient activation of both humoral and cellular immunity, a prerequisite for successful virus neutralization and/or elimination (such as SARS-COV-2) (Pulendran et al., 2021, Nature Reviews Drug Discovery, 20,454-475).

Recently, it has been shown that targeting mitochondrial metabolism to boost T cell memory formation and metabolic fitness might represent an attractive strategy to improve cancer immunotherapy including CAR-T therapy (Li et al. 2020, Front Immunol., 11:1834).

Pyruvate is a metabolite involved in a number of biological processes and is particularly critical in cellular respiration. End-product of glycolysis in the cytosol, pyruvate needs to enter the mitochondria to fuel the Krebs cycle and to boost oxidative phosphorylation and ATP production. To enter mitochondria, pyruvate crosses the outer mitochondrial membrane to reach the intermembrane space, probably through the large, relatively non-specific, voltage-dependent anion channel, and it is then transported together with a proton across the inner mitochondrial membrane by the mitochondrial pyruvate carrier (MPC) (Papa et al., 1971, FEBS Lett., 12, 285 288). The existence of MPC was proposed on theoretical grounds several decades ago, although the molecular identification of the MPC complex was only achieved in 2012 (Herzig et al., 2012, Science, 337, 93 96). As the sole point of entry for pyruvate into the mitochondrial matrix, the MPC plays a crucial role in coordinating glycolytic and mitochondrial activities, and it provides a key decision point for modulating cellular energy production and metabolism.

It has been recently reported that the MPC plays a pivotal role in many physiological and pathological processes across the human lifespan, from embryonic development to aging associated neurodegeneration (Buchanan et al., 2020, Biomolecules, 10 (8): 1162; Zangari et al., 2020, Biomolecules, 10 (7): 1068). In particular, MPC is of importance in Cancer Cell Metabolism and Tumorigenesis (Ruiz Iglesias et al., 2021, Cancers 2021, 13, 148).

Especially, it has been shown that genetic inhibition of the MPC stimulates proliferation of stem cells in various tissues, including intestine and skin (Flores et al., 2021, Exp Dermatol., 30 (4): 448-456; Schell et al., 2017, Nat Cell Biol., 19 (9): 1027 1036).

Further, it has been shown that genetic inhibition of the MPC could be an interesting therapeutic strategy to treat metabolic diseases including type 2 diabetes (Hojlund et al., 2008, Endocrinol Metab Clin North Am., 37 (3): 713-3) and Non-Alcoholic Steato Hepatitis (NASH), an increasingly common liver disease (Harisson et al., 2020, J. Hepatol., 72, 613 626), in cancer and to potentiate existing cancer treatments including radiotherapy ((orbet et al., 2018, Nat. Commun., 9, 1208).

Therefore, in view of the recent developments of various strategies in cancer immunotherapy such as cancer vaccines, adoptive cellular immunotherapy, immune checkpoint blockade and oncolytic viruses but also the encountered limitations to their efficacy, there is a growing need of developing efficient anti-cancer therapies for solid tumor cancers, in particular for cancers prone for developing a resistance to immunotherapy which would potentiate cancer vaccine treatments. Further, there is the need for the developement of efficient MPC inhibitors in order to further understand the role MPC activity in various disorders.

### SUMMARY OF THE INVENTION

The present invention is directed towards the unexpected findings that pharmacological MPC inhibition with compounds of the invention during T cell culture for the preparation of ACT products can induce an increase in the proportion of activated T cells committed to acquire a memory phenotype and improved anti-tumoral activity. Therefore, the MPC inhibitors according to the invention are considered to be of high interest for specific immunotherapy of cancer, in particular in the adoptive T cell transfer approach (ACT), especially for CAR T therapy, and/or for cancer vaccines and/or vaccines against infectious diseases.

The present invention is directed to compositions and methods useful for inhibiting the activity of the MPC and therefore in particular for enhancing the efficacy and durability of cancer treatments including ACT, for stimulating the proliferation of stem cells in various tissues, including intestine, skin and brain, for decreasing inflammation and fibrosis of several organs including the liver, lungs, pancreas, muscles, to decrease the development of tumors and/or to potentiate existing cancer treatments including immune checkpoint inhibitors and radiotherapy.

A first aspect of the invention provides a compound of the invention for the prevention and/or treatment of a disease or disorder, wherein said disease or disorder is selected from a cancer, an auto-immune disease such as multiple sclerosis, a metabolic diseases such as type 2 diabetes, an hair loss disorder such as alopecia, a neurodegenerative disorder such as Parkinson or Alzheimer's disease, a fibrotic disease such as pulmonary fibrosis or non alcoholic steatohepatitis (NASH), a skin or tissue injury such a skin wound or a burn, and an acute pathology of the brain such as stroke or brain trauma.

Another aspect of the invention provides a compound of the invention for use in the regeneration of the skin or a tissue.

Another aspect of the invention provides a use of one or more compounds of the invention for the preparation of a pharmaceutical composition for the prevention and/or treatment of a disease or disorder, wherein said disease or disorder is selected from a cancer, an auto-immune disease such as multiple sclerosis, a metabolic diseases such as type 2 diabetes, an hair loss disorder such as alopecia, a neurodegenerative disorder such as Parkinson or Alzheimer's disease, a fibrotic disease such as pulmonary fibrosis or non alcoholic steatohepatitis (NASH), a skin or tissue injury such a skin wound or a burn, and an acute pathology of the brain such as stroke or brain trauma.

Another aspect of the invention relates to a pharmaceutical composition containing at least one compound according to the invention, as well as tautomers, geometrical isomers, optically active forms and pharmaceutically acceptable salts thereof combined with at least one anti-cancer immunotherapeutic agent such as CAR T-cells or an immune checkpoint inhibitor and at least one pharmaceutically acceptable carrier, diluent or excipient thereof.

Another aspect of the invention relates to a method for treating a subject suffering from a disease or disorder, wherein said disease or disorder is selected from a cancer, an auto-immune disease such as multiple sclerosis, a metabolic diseases such as type 2 diabetes, an hair loss disorder such as alopecia, a neurodegenerative disorder such as Parkinson or Alzheimer's disease, a fibrotic disease such as pulmonary fibrosis or non alcoholic steatohepatitis (NASH), a skin or tissue injury such a skin wound or a burn, and an acute pathology of the brain such as stroke or brain trauma, said method comprising administering an effective amount of one or more compound of the invention in a subject in need thereof.

Another aspect of the invention relates to MPC inhibitors described, their pharmaceutical formulations and their use as a medicament.

Another aspect of the invention relates to an in vitro method for generating and/or maintaining T-cells with a memory phenotype.

Other features and advantages of the invention will be apparent from the following detailed description.

### DESCRIPTION OF THE FIGURES

FIG. 1 represents the treatment protocol (A) and the effects of the in vitro treatment of mouse CD8 T cells with a compound inhibiting the mitochondrial pyruvate carrier (MPCi) on memory characteristics (B to C) as described in Example 3. (A) Schematic representation of the in vitro mouse CD8 T cell activation and treatment. (B—C) FACS analysis at day 7 showing the percentage of cells positive for the memory markers CD62L (B) and CD127 (C). * p<0.05, ** p>0.01, *** p>0.01. Data represents mean #s.e.m.

FIG. 2 represents the treatment protocol (A) and the antitumoral activity (B to S) of a compound of the invention (C45) in a mouse melanoma model as described in Example 4. (B—C) B16-OVA tumor growth (B) and weight (C) in mice upon transfer of Compound 45- or DMSO-treated cells, or in untreated mice. (D-G) Blood analysis day 9 post-ACT showing percentages of transferred cells (D), short-lived effector cells (E), memory precursor cells (F) and central memory cells (G); (H—K) Analysis of tumor infiltrating T cells, showing percentage of transferred cells (H), exhausted cells (I), terminally exhausted cells (J) and progenitor exhausted cells (K). (L-N) Analysis of the spleen showing percentage of transferred cells (L), central memory T cells (M) and T cells expressing TCF1 (N). (O—S) Single-cell suspension of tumors re-stimulated with Ovalbubin N4 peptide for 4 h. IFNγ (O), TNFα (P), IL2 (Q), Granzyme B (R) and CD107a(S) expression was measured by flow cytometry. ns: non significant, *p<0.05, ** p>0.01, *** p>0.001. Data represents mean #s.e.m.

FIG. 3 represents the treatment protocol (A) and the antitumoral activity (B to 0) of a compound of the invention (C45) in a mouse melanoma model as described in Example 4 (transferring 2×10⁶ DMSO- or Compound 45-treated OT1 T cells, instead of only 10⁵). (B—C) B16-OVA tumor growth (B) and weight (C) in mice upon transfer of Compound 45- or DMSO-treated cells, or in untreated mice. (D-G) Analysis of tumor infiltrating T cells, showing number of transferred cells (D), exhausted cells (E), terminally exhausted cells (F) and progenitor exhausted cells (G). (H-J) Analysis of the spleen showing number of transferred cells (H), central memory T cells (I) and T cells expressing TCF1 (J). (K—O) Single-cell suspension of tumors was re-stimulated with N4 peptide for 4 h. IFNγ (K), TNFα (L), IL2 (M), Granzyme B (N) and CD107a (O) expression was measured by flow cytometry. ns: non significant, *p<0.05, ** p>0.01, *** p>0.001. Data represents mean #s.e.m.

FIGS. 4 and 5 represent the experimental design (4A) and the effects (4B-F and 5A-K) of a compound of the invention during the production of murine CAR T cells improves their memory phenotype and antitumor function upon adoptive cell transfer therapy as described in Example 5. (4B) Tumor growth curve; (4C) Tumor weight at the time of dissection; (4D) Number of Her2-CAR T cells per μl of blood; (4E and F) Percentages of short-lived effector cells (4E) and memory precursor cells (4F) out of Her2-CAR T cells.ns: non significant, *p<0.05, ** p>0.01. Data represents mean+s.e.m. (5A) Number of Her2-CAR T cells per tumor-draining lymph node; (5B) Percentage of central memory T cells out of Her2-CAR T cells in the lymph node; (5C) Percentage of TCF1-positive cells out of Her2-CAR T cells in the lymph node; (5D) Number of Her2-CAR T cells per spleen; (5E) Percentage of central memory T cells out of Her2-CAR T cells in the spleen; (5F) Percentage of TCF1-positive cells out of Her2-CAR T cells in the spleen; (5G) Number of Her2-CAR T cells per mg of tumor. (5H) Percentage of TCF1-positive cells out of Her2-CAR T cells in the tumor; (5I and J) Percentages of progenitor-exhausted T cells (I) and terminally exhausted T cells (5J) out of Her2-CAR T cells in the tumor; (5K) Percentage of exhaustion marker (PD1 and TIM3)-expressing Her2-CAR T cells in the tumor. ns: non significant, *p<0.05, ** p>0.01, *** p<0.001. Data represents mean #s.e.m.

### DETAILED DESCRIPTION OF THE INVENTION

The expression “solid tumour cancer” includes glioblastoma, lung cancer (small cell and non-small cell), breast cancer, ovarian cancer, cervical cancer, uterus cancer, head and neck cancer, melanoma, hepatocellular carcinoma, colon cancer, rectal cancer, colorectal carcinoma, kidney cancer, prostate cancer, gastric cancer, bronchus cancer, pancreatic cancer, urinary bladder cancer, hepatic cancer and brain cancer, in particular glioblastoma.

The expression “liquid tumour cancer” includes lymphomas and leukemias.

The expression “immunotherapeutic agent” refers to agents which supporttheimmune system to fight a disease such as cancer. There are currently four major categories: T-cell therapies, immune checkpoint inhibitors, monoclonal antibodies and cancer vaccines.

As used herein, “treatment” and “treating” and the like generally mean obtaining a desired pharmacological and physiological effect. The term “treatment” as used herein covers any treatment of a disease in a mammal, particularly a human, and includes inhibiting the disease, i.e., arresting its development; or relieving the disease, i.e. causing regression of the disease and/or its symptoms or conditions such as tumor growth arrest or tumor regression.

The term “subject” as used herein refers to mammals. For examples, mammals contemplated by the present invention include human, primates, domesticated animals such as cattle, sheep, pigs, horses, laboratory rodents, dogs and the like.

The term “effective amount” as used herein refers to an amount of at least one particle or a pharmaceutical formulation thereof according to the invention that elicits the biological or medicinal response in a tissue, system, animal, or human that is being sought. In one embodiment, the effective amount is a “therapeutically effective amount” for the alleviation of the symptoms of the disease or condition being treated. Typically, an effective amount can be used to inhibit the growth of cancer cells, i.e. any slowing of the rate of cancer cell proliferation and/or migration, arrest of cancer cell proliferation and/or migration, or killing of cancer cells, such that the rate of cancer cell growth is reduced in comparison with the observed or predicted rate of growth of an untreated control cancer cell. The term “inhibits growth” can also refer to a reduction in size or disappearance of a cancer cell or tumor, as well as to a reduction in its metastatic potential. Preferably, such an inhibition at the cellular level may reduce the size, defer the growth, reduce the aggressiveness, or prevent or inhibit metastasis of a cancer in a patient. Those skilled in the art can readily determine, by any of a variety of suitable indicia, whether cancer cell growth is inhibited.

The term “efficacy” of a treatment according to the invention can be measured based on changes in the course of a disease in response to a use or a method according to the invention. The efficacy of a treatment of a cancer according to the invention can be measured by a reduction of tumour volume, and/or an increase of progression free survival time and/or increased health and well-being of the subject (e.g. repressing a cancer). Inhibition of cancer cell growth may be evidenced, for example, by arrest of cancer cells in a particular phase of the cell cycle, e.g., arrest at the G2/M phase of the cell cycle. Inhibition of cancer cell growth can also be evidenced using well known imaging methods such as magnetic resonance imaging, computerized axial tomography, PET, SPECT, photo-acoustic imaging, X-rays and fluorescence imaging/detection. Cancer cell growth can also be determined indirectly, for example by determining the levels of circulating carcino-embryonic antigen, prostate specific antigen or other cancer-specific antigens that are correlated with cancer cell growth.

In particular, efficacy of a combined treatment according to the invention can be assessed by reduction of tumour size, or disappearance of tumour or of any biomarker relevant for a cancer type. In the context of Adoptive Cell Transfer (ACT), efficacy can also be measured by assessing transferred T-cell infiltration into the tumour, transferred T-cell migration into lymph nodes or any changes in T-cell “profile” or “differentiation status”.

The term “profile” of a cell, in particular a T-cell or any Peripheral Blood Mononuclear Cell (PBMC), according to the invention can be measured based on changes in gene expression or cell surface markers in response and/or an increased basal oxygen consumption, maximal respiratory capacity and/or spare respiratory capacity compared to a control. to a use or a method according to the invention.

More specifically the term “memory phenotype”, as used herein, is defined as a cell state which resembles a memory T-cell at least in some aspects. The term “memory-like T-cell” is used herein interchangeably with the term “memory phenotype”. An important feature associated with a memory phenotype is the longevity of the cell. Longevity means that the cell or a progenitor survives long enough, e.g. without dividing or slowly dividing, in a subject to be able to elicit a therapeutic effect. In particular, a cell with a memory phenotype has stem cell-like properties. The longevity is preferably due to self-renewal which comprises proliferation. Self-renewal, as used herein, is not meant in a strict sense, but also includes the capacity to maintain a largely similar, although not necessarily identical, phenotype for a therapeutically relevant period of time. The self-renewal can be maintained for the entire life-time or even beyond, but it is sufficient, as used herein, if it is maintained long enough for the therapeutic purpose. A therapeutically relevant period of time means that the transferred cells or their progeny persist long enough in a subject to have a therapeutic effect. While a T-cell with a memory phenotype is living, and preferably proliferating, it typically maintains the capacity to differentiate into effector cells. The capacity to generate an effector T-cell is thus another important feature associated with a memory phenotype. A T-cell with a memory phenotype thus can produce a higher number of therapeutically active effector cells than effector cells themselves which rather tend to senesce and die early. Important functional features associated with a memory phenotype can be also measured relative to other cell populations. For example, longevity, self-renewal and/or the capacity to differentiate into effector cells may be compared to effector cells, terminally differentiated cells and/or senescent cells.

Suitable positive expression markers for the memory phenotype (memory markers), in particular of CD8+ T-cells, but also, at least partly, of CD4+ T-cells and/or B-cells, are, for example, CCR7, CD62L, CD27, CD28, CD127 and/or TCF1.

Another important feature associated with the memory phenotype is the ability of the cells to react with an increased amplitude of (re) activation to a reencounter of the antigen, as is observed, e.g., with memory T-cells.

Unless otherwise constrained by the definition of the individual substituent, the term “substituted” refers to groups substituted with from 1 to 5 substituents selected from the group consisting of “C₁-C₆ alkyl,” “C₂-C₆ alkenyl,” “C₂-C₆ alkynyl,” “C₃-C₈-cycloalkyl,” “heterocycloalkyl,” “C₁-C₆ alkyl aryl,” “C₁-C₆ alkyl heteroaryl,” “C₁-C₆ alkyl cycloalkyl,” “C₁-C₆ alkyl heterocycloalkyl,” “amino,” “alkyl amino,” “aryl amino”, “heteroaryl amino” and “aryl oxy”, “heteroaryl oxy”, “urea” “aminosulfonyl,” “ammonium,” “alkoxy,” “acyl”, “acyl amino,” “amino carbonyl,” “aryl,” “heteroaryl,” “sulfinyl,” “sulfonyl,” “sulphonamide”, “alkoxy,” “alkoxy carbonyl,” “carbamate,” “sulfanyl,” “halogen,” trihalomethyl, cyano, hydroxy, mercapto, nitro, and the like.

The term “pharmaceutically acceptable salts or complexes” refers to salts or complexes of the below-specified compounds of the invention. Examples of such salts include, but are not restricted, to base addition salts formed by reaction of compounds of the invention with organic or inorganic bases such as hydroxide, carbonate, bicarbonate or the like, of a metal cation such as those selected in the group consisting of alkali metals (sodium, potassium or lithium), alkaline earth metals (e.g. calcium or magnesium), or with an organic primary, secondary or tertiary alkyl amine. Other examples of such salts include, but are not restricted, to acid addition salts formed by reaction of compounds of the invention with organic or inorganic acids such as hydrochloric acid, hydrobromic acid, sulphuric acid, para-toluene sulfonic acid, 2-naphtalene sulfonic acid, camphosulfonic acid, benzene sulfonic acid, oxalic acid or the like.

Compounds of the invention also include isotopic isomers such as deuterated and C13 analogues of compounds of Formula (I).

“Pharmaceutically active derivative” refers to any compound that upon administration to the recipient is capable of providing directly or indirectly, the activity disclosed herein.

### Compounds According to the Invention

In one embodiment, the invention provides a compound of Formula (I)

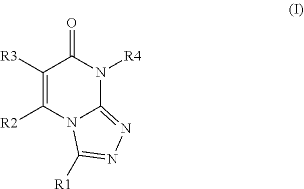

wherein R1 is a moiety R5-R6; R2 is selected from H, optionally substituted C₁-C₆ alkyl (such as optionally substituted methyl, optionally substituted ethyl, optionally substituted propyl such as trifluoro propyl, optionally substituted butyl, optionally substituted t-butyl), optionally substituted aryl, optionally substituted heteroaryl, optionally substituted aryl C₁-C₆ alkyl (optionally substituted aryl methyl such as optionally substituted phenyl methyl), optionally substituted heteroaryl C₁-C₆ alkyl, optionally substituted C₁-C₆ alkoxy such as optionally substituted C₁-C₆ alkoxy substituted C₁-C₆ alkyl, such as for example optionally substituted ethoxy (e.g. optionally substituted ethoxy benzyl such as optionally substituted ethoxy halogeno benzyl, i.e. 2-chlorobenzyl oxy ethyl or 3-chlorobenzyl oxy ethyl, 4-chlorobenzyl oxy ethyl or optionally substituted ethoxy methyl), optionally substituted methoxy (e.g. optionally substituted methoxy methyl), optionally substituted C₃-C₈ heterocycloalkyl and optionally substituted C₃-C₈ cycloalkyl such as optionally substituted cyclopropyl; R3 is selected from H and optionally substituted C₁-C₆ alkyl (e.g. optionally substituted methyl) and wherein at least one of R2 and R3 is not H; R4 is H; R5 is selected from a bond, S, SO₂, NR9 and O; R6 is a group —(CR10R11)ₙ—R7 wherein n is an integer between 0 and 2; R7 is selected from optionally substituted C₁-C₆ alkyl (e.g. optionally substituted methyl, optionally substituted propyl, optionally substituted t-butyl), optionally substituted C₂-C₆ alkenyl (e.g. 3-phenyl propylenyl), optionally substituted heterocycle, optionally substituted aryl such as optionally substituted phenyl (e.g. 4-nitrophenyl, 2-nitrophenyl, 3-nitrophenyl, 4-nitrophenyl, 2-fluorophenyl, 3-fluorophenyl, 2,5-difluorophenyl, 2,4-difluorophenyl, 3,5-difluorophenyl, 2,4-difluoro phenyl, 2,6-difluoro phenyl, 3-(difluoromethyl) phenyl, 2-(difluoromethyl) phenyl, 4-(difluoromethyl) phenyl, 3,4-difluoro phenyl, 2-chloro-6-fluorophenyl, 2-chlorophenyl, 2-chloro-5-fluoro phenyl, 3-chloro-5-fluoro phenyl, 4-chloro-3-fluoro phenyl, 3-chlorophenyl, 2,4-dichloro phenyl, 3,4-dichloro phenyl, 4-chlorophenyl, 5-fluoro-2-methylphenyl, 4-fluoro-2-benzonitrile, 2,5-dimethylphenyl, 3-methyl phenyl, 4-methyl phenyl, 3-(trifluoromethyl) phenyl, 4-(trifluoromethyl) phenyl, 5-fluoro-2-methoxy phenyl, 2-methoxy phenyl, 3-methoxy phenyl, 4-methoxy phenyl, 4-bromo phenyl), optionally substituted naphthalenyl (e.g. optionally substituted naphthalen-1-yl), optionally substituted heteroaryl such as optionally substituted furanyl (e.g. optionally substituted furan-3-yl such as furanyl, methoxy carbonyl furanyl), optionally substituted pyrazolyl, optionally substituted pyridinyl (e.g. optionally substituted pyridine-4-yl), optionally substituted benzofuranyl (e.g. 1-benzofuranyl), cyano and —C(O)—R8; R8 is selected from optionally substituted amino (e.g. optionally substituted 4-ethoxyphenyl amino), optionally substituted alkoxy (e.g. optionally substituted methoxy, optionally substituted ethoxy) and optionally substituted aryl (e.g. optionally substituted phenyl such as phenyl, 2-methoxy phenyl, 2-chloro-6-fluorophenyl); R9 is H or optionally substituted C₁-C₆ alkyl (e.g. optionally substituted methyl, optionally substituted ethyl); R10 and R11 are independently selected from H and optionally substituted C₁-C₆ alkyl (e.g. optionally substituted methyl, optionally substituted ethyl); as well as tautomers, geometrical isomers, optically active forms and pharmaceutically acceptable salts and pharmaceutically active derivative thereof for use in the prevention and/or treatment of a disease or disorder, wherein said disease or disorder is selected from a cancer, an auto-immune disease such as multiple sclerosis, a metabolic diseases such as type 2 diabetes, an hair loss disorder such as alopecia, a neurodegenerative disorder such as Parkinson or Alzheimer's disease, a fibrotic disease such as pulmonary fibrosis or non alcoholic steatohepatitis (NASH), a skin or tissue injury such a skin wound or a burn, and an acute pathology of the brain such as stroke or brain trauma or for use in the regeneration of the skin or a tissue.

According to a particular embodiment are provided isotopic isomers such as deuterated and C13 analogues of compounds of Formula (I).

According to a particular aspect, is provided a compound of Formula (I) wherein R1 is a moiety S—R6.

According to a particular aspect, is provided a compound of Formula (I) wherein R1 is a moiety NR9-R6.

According to a particular aspect, is provided a compound of Formula (I) wherein R1 is a moiety NH—R6.

According to a particular aspect, is provided a compound of Formula (I) wherein R1 is a moiety R6.

According to a particular aspect, is provided a compound of Formula (I) wherein R2 is optionally substituted C₁-C₆ alkyl.

According to a particular aspect, is provided a compound of Formula (I) wherein R2 is optionally substituted propyl.

According to a particular aspect, is provided a compound of Formula (I) wherein R3 is optionally substituted C₁-C₆ alkyl.

According to a particular aspect, is provided a compound of Formula (I) wherein R3 is optionally substituted methyl.

According to a particular aspect, is provided a compound of Formula (I) wherein R3 is H.

According to a particular aspect, is provided a compound of Formula (I) wherein n is 0.

According to a particular aspect, is provided a compound of Formula (I) wherein n is 1.

According to a particular aspect, is provided a compound of Formula (I) wherein n is 2.

According to a particular aspect, is provided a compound of Formula (I) wherein R7 is optionally substituted C₁-C₆ alkyl.

According to a particular aspect, is provided a compound of Formula (I) wherein R7 is optionally substituted C₂-C₆ alkenyl.

According to a particular aspect, is provided a compound of Formula (I) wherein R7 is an optionally substituted aryl.

According to a particular aspect, is provided a compound of Formula (I) wherein R7 is an optionally substituted phenyl.

According to a particular aspect, is provided a compound of Formula (I) wherein R7 is an optionally substituted heteroaryl.

According to a particular aspect, is provided a compound of Formula (I) wherein R7 is an optionally substituted furanyl.

According to a particular aspect, is provided a compound of Formula (I) wherein R7 is an optionally substituted pyrazolyl.

According to a particular aspect, is provided a compound of Formula (I) wherein R7 is an optionally substituted pyridinyl.

According to a particular aspect, is provided a compound of Formula (I) wherein R7 is cyano.

According to a particular aspect, is provided a compound of Formula (I) wherein R9 is H.

According to a particular aspect, is provided a compound of Formula (I) wherein R10 is H.

According to a particular aspect, is provided a compound of Formula (I) wherein R10 is optionally substituted C₁-C₆ alkyl.

According to a particular aspect, is provided a compound of Formula (I) wherein R11 is H.

According to a particular aspect, is provided a compound of Formula (I) wherein n is an integer selected from 0 and 1, R5 is S and R7 is an optionally substituted aryl (e.g. optionally substituted phenyl).

According to a particular aspect, is provided a compound of Formula (I) wherein said compound has an inhibitory activity against MPC.

In a further particular embodiment, compounds of the present invention include in particular those selected from the following group:
3-[(3,4-difluorobenzyl)sulfanyl]-5-propyl[1,2,4]triazolo[4,3-a]pyrimidin-7 (8H)-one (1);
3-[(2-chloro-5-fluorobenzyl)sulfanyl]-5-propyl[1,2,4]triazolo[4,3-a]pyrimidin-7 (8H)-one (2);
3-[(3,5-difluorobenzyl)sulfanyl]-5-propyl[1,2,4]triazolo[4,3-a]pyrimidin-7 (8H)-one (3);
3-{[3-(difluoromethyl)benzyl]sulfanyl}-5-propyl[1,2,4]triazolo[4,3-a]pyrimidin-7 (8H)-one (4);
3-{[2-(difluoromethyl)benzyl]sulfanyl}-5-propyl[1,2,4]triazolo[4,3-a]pyrimidin-7 (8H)-one (5);
3-{[4-(difluoromethyl)benzyl]sulfanyl}-5-propyl[1,2,4]triazolo[4,3-a]pyrimidin-7 (8H)-one (6);
3-{[(7-oxo-5-propyl-7,8-dihydro[1,2,4]triazolo[4,3-a]pyrimidin-3-yl)sulfanyl]methyl}benzonitrile (7);
4-fluoro-2-{[(7-oxo-5-propyl-7,8-dihydro[1,2,4]triazolo[4,3-a]pyrimidin-3-yl)sulfanyl]methyl}benzonitrile (8);
3-[(5-fluoro-2-methoxybenzyl)sulfanyl]-5-propyl[1,2,4]triazolo[4,3-a]pyrimidin-7 (8H)-one (9);
3-[(3-chloro-5-fluorobenzyl)sulfanyl]-5-propyl[1,2,4]triazolo[4,3-a]pyrimidin-7 (8H)-one (10);
3-[(4-chloro-3-fluorobenzyl)sulfanyl]-5-propyl[1,2,4]triazolo[4,3-a]pyrimidin-7 (8H)-one (11);
3-[(5-fluoro-2-methylbenzyl)sulfanyl]-5-propyl[1,2,4]triazolo[4,3-a]pyrimidin-7 (8H)-one (12);
3-[(4-fluoro-2-methylbenzyl)sulfanyl]-5-propyl[1,2,4]triazolo[4,3-a]pyrimidin-7 (8H)-one (13);
3-[(2-phenylethyl)sulfanyl]-5-propyl[1,2,4]triazolo[4,3-a]pyrimidin-7 (8H)-one (14);
3-[(furan-3-ylmethyl)sulfanyl]-5-propyl[1,2,4]triazolo[4,3-a]pyrimidin-7 (8H)-one (15);
3-[(2-methoxybenzyl)sulfanyl]-5-propyl[1,2,4]triazolo[4,3-a]pyrimidin-7 (8H)-one (16);
3-[(3-methoxybenzyl)sulfanyl]-5-propyl[1,2,4]triazolo[4,3-a]pyrimidin-7 (8H)-one (17);
4-{[(7-oxo-5-propyl-7,8-dihydro[1,2,4]triazolo[4,3-a]pyrimidin-3-yl)sulfanyl]methyl}benzonitrile (18);
3-[(1-benzofuran-5-ylmethyl)sulfanyl]-5-propyl[1,2,4]triazolo[4,3-a]pyrimidin-7 (8H)-one (19);
3-[(4-methoxybenzyl)sulfanyl]-5-propyl[1,2,4]triazolo[4,3-a]pyrimidin-7 (8H)-one (20);
3-[(2,5-difluorobenzyl)sulfanyl]-5-propyl[1,2,4]triazolo[4,3-a]pyrimidin-7 (8H)-one (21);
3-[(2,4-difluorobenzyl)sulfanyl]-5-propyl[1,2,4]triazolo[4,3-a]pyrimidin-7 (8H)-one (22);
3-[(2,6-difluorobenzyl)sulfanyl]-5-propyl[1,2,4]triazolo[4,3-a]pyrimidin-7 (8H)-one (23);
5-propyl-3-[(1H-pyrazol-4-ylmethyl)sulfanyl][1,2,4]triazolo[4,3-a]pyrimidin-7 (8H)-one (24);
3-[(2,6-dimethylbenzyl)sulfanyl]-5-propyl[1,2,4]triazolo[4,3-a]pyrimidin-7 (8H)-one (25);
3-[(2-chloro-6-fluorobenzyl)sulfanyl]-5-propyl[1,2,4]triazolo[4,3-a]pyrimidin-7 (8H)-one (26);
3-[(2,5-dimethylbenzyl)sulfanyl]-5-propyl[1,2,4]triazolo[4,3-a]pyrimidin-7 (8H)-one (27);
3-{[2-(2,4-difluorophenyl)ethyl]sulfanyl}-5-propyl[1,2,4]triazolo[4,3-a]pyrimidin-7 (8H)-one (28);
3-[(2,5-difluorobenzyl)sulfanyl]-5-methyl[1,2,4]triazolo[4,3-a]pyrimidin-7 (8H)-one (29);
5-[2-(benzyloxy)ethyl]-3-[(2,5-difluorobenzyl)sulfanyl][1,2,4]triazolo[4,3-a]pyrimidin-7 (8H)-one (30);
3-[(2,5-difluorobenzyl)sulfanyl]-5-(methoxymethyl) [1,2,4]triazolo[4,3-a]pyrimidin-7 (8H)-one (31);
5-{2-[(4-chlorobenzyl)oxy]ethyl}-3-[(2,5-difluorobenzyl)sulfanyl][1,2,4]triazolo[4,3-a]pyrimidin-7 (8H)-one (32);
5-{2-[(2-chlorobenzyl)oxy]ethyl}-3-[(2,5-difluorobenzyl)sulfanyl][1,2,4]triazolo[4,3-a]pyrimidin-7 (8H)-one (33);
5-{2-[(3-chlorobenzyl)oxy]ethyl}-3-[(2,5-difluorobenzyl)sulfanyl][1,2,4]triazolo[4,3-a]pyrimidin-7 (8H)-one (34);
3-[(2,5-difluorobenzyl)sulfanyl]-5-(2-methoxyethyl) [1,2,4]triazolo[4,3-a]pyrimidin-7 (8H)-one (35);
3-[(2-chloro-6-fluorobenzyl)sulfanyl]-5-methyl[1,2,4]triazolo[4,3-a]pyrimidin-7 (8H)-one (36);
3-[(2-chloro-6-fluorobenzyl)sulfanyl]-5-ethyl[1,2,4]triazolo[4,3-a]pyrimidin-7 (8H)-one (37);
5-butyl-3-[(2-chloro-6-fluorobenzyl)sulfanyl][1,2,4]triazolo[4,3-a]pyrimidin-7 (8H)-one (38);
3-[(2-chloro-6-fluorobenzyl)sulfanyl]-5-cyclopropyl[1,2,4]triazolo[4,3-a]pyrimidin-7 (8H)-one (39);
3-[(2-chloro-6-fluorobenzyl)sulfanyl]-5-(2-methylpropyl) [1,2,4]triazolo[4,3-a]pyrimidin-7 (8H)-one (40);
3-[(2-chloro-6-fluorobenzyl)sulfanyl]-5-(cyclopropylmethyl) [1,2,4]triazolo[4,3-a]pyrimidin-7 (8H)-one (41);
3-[(2-chloro-6-fluorobenzyl)sulfanyl]-5-(methoxymethyl) [1,2,4]triazolo[4,3-a]pyrimidin-7 (8H)-one (42);
5-[2-(benzyloxy)ethyl]-3-[(2-chloro-6-fluorobenzyl)sulfanyl][1,2,4]triazolo[4,3-a]pyrimidin-7 (1H)-one (43);
3-[(2-chloro-6-fluorobenzyl)sulfanyl]-5-(3,3,3-trifluoropropyl) [1,2,4]triazolo[4,3-a]pyrimidin-7 (8H)-one (44);
5-benzyl-3-[(2-chloro-6-fluorobenzyl)sulfanyl][1,2,4]triazolo[4,3-a]pyrimidin-7 (8H)-one (45);
4-{[(5-benzyl-7-oxo-7,8-dihydro[1,2,4]triazolo[4,3-a]pyrimidin-3-yl)sulfanyl]methyl}benzonitrile (46);
3-(phenylsulfanyl)-5-propyl[1,2,4]triazolo[4,3-a]pyrimidin-7 (8H)-one (47);
3-[(2-chloro-6-fluorobenzyl)sulfanyl]-6-methyl-5-propyl[1,2,4]triazolo[4,3-a]pyrimidin-7 (8H)-one (48);
3-[(2-chloro-6-fluorobenzyl)amino]-5-propyl[1,2,4]triazolo[4,3-a]pyrimidin-7 (8H)-one (49);
3-[(2-chloro-6-fluorobenzyl)sulfonyl]-5-propyl[1,2,4]triazolo[4,3-a]pyrimidin-7 (8H)-one (50);
3-(2-phenylethyl)-5-propyl[1,2,4]triazolo[4,3-a]pyrimidin-7 (8H)-one (51);
3-[(2-chlorobenzyl)sulfanyl]-5-methyl[1,2,4]triazolo[4,3-a]pyrimidin-7 (8H)-one (52);
5-methyl-3-(methylsulfanyl) [1,2,4]triazolo[4,3-a]pyrimidin-7 (8H)-one (53);
5-methyl-3-[(4-nitrobenzyl)sulfanyl][1,2,4]triazolo[4,3-a]pyrimidin-7 (8H)-one (54);
3-{[(2E)-3-phenylprop-2-en-1-yl]sulfanyl}-5-propyl[1,2,4]triazolo[4,3-a]pyrimidin-7 (8H)-one (55);
N-(4-ethoxyphenyl)-2-[(7-oxo-5-propyl-7,8-dihydro[1,2,4]triazolo[4,3-a]pyrimidin-3-yl) sulfanyl]acetamide (56);
methyl5-{[(7-oxo-5-propyl-7,8-dihydro[1,2,4]triazolo[4,3-a]pyrimidin-3-yl)sulfanyl]
methyl}furan-2-carboxylate (57);
ethyl 2-[(7-oxo-5-propyl-7,8-dihydro[1,2,4]triazolo[4,3-a]pyrimidin-3-yl)sulfanyl]propanoate (58);
ethyl2-[(7-oxo-5-propyl-7,8-dihydro[1,2,4]triazolo[4,3-a]pyrimidin-3-yl)sulfanyl]butanoate (59);
methyl2-[(7-oxo-5-propyl-7,8-dihydro[1,2,4]triazolo[4,3-a]pyrimidin-3-yl)sulfanyl]butanoate (60);
methyl2-[(7-oxo-5-propyl-7,8-dihydro[1,2,4]triazolo[4,3-a]pyrimidin-3-yl)sulfanyl]propanoate (61);
benzyl[(7-oxo-5-propyl-7,8-dihydro[1,2,4]triazolo[4,3-a]pyrimidin-3-yl)sulfanyl]acetate (62);
[(7-oxo-5-propyl-7,8-dihydro[1,2,4]triazolo[4,3-a]pyrimidin-3-yl)sulfanyl]acetonitrile (63);
3-[(2-oxo-2-phenylethyl)sulfanyl]-5-propyl[1,2,4]triazolo[4,3-a]pyrimidin-7 (8H)-one (64);
3-{[2-(4-methoxyphenyl)-2-oxoethyl]sulfanyl}-5-propyl[1,2,4]triazolo[4,3-a]pyrimidin-7 (8H)-one (65);
N-(5-chloro-2-methoxyphenyl)-2-[(7-oxo-5-propyl-7,8-dihydro[1,2,4]triazolo[4,3-a]pyrimidin-3-yl)sulfanyl]acetamide (66);
5,6-dimethyl-3-(propylsulfanyl) [1,2,4]triazolo[4,3-a]pyrimidin-7 (8H)-one (67);
5,6-dimethyl-3-[(3-methylbutyl)sulfanyl][1,2,4]triazolo[4,3-a]pyrimidin-7 (8H)-one (68);
5,6-dimethyl-3-[(4-nitrobenzyl)sulfanyl][1,2,4]triazolo[4,3-a]pyrimidin-7 (8H)-one (69);
5,6-dimethyl-3-[(3-methylbenzyl)sulfanyl][1,2,4]triazolo[4,3-a]pyrimidin-7 (8H)-one (70);
3-[(2,5-dimethylbenzyl)sulfanyl]-5,6-dimethyl[1,2,4]triazolo[4,3-a]pyrimidin-7 (8H)-one (71);
5,6-dimethyl-3-[(4-methylbenzyl)sulfanyl][1,2,4]triazolo[4,3-a]pyrimidin-7 (8H)-one (72);
5,6-dimethyl-3-[(3-nitrobenzyl)sulfanyl][1,2,4]triazolo[4,3-a]pyrimidin-7 (8H)-one (73);
3-[(4-chlorobenzyl)sulfanyl]-5,6-dimethyl[1,2,4]triazolo[4,3-a]pyrimidin-7 (8H)-one (74);
3-[(2-chlorobenzyl)sulfanyl]-5,6-dimethyl[1,2,4]triazolo[4,3-a]pyrimidin-7 (8H)-one (75);
5,6-dimethyl-3-{[3-(trifluoromethyl)benzyl]sulfanyl}[1,2,4]triazolo[4,3-a]pyrimidin-7 (8H)-one (76);
5,6-dimethyl-3-{[4-(trifluoromethyl)benzyl]sulfanyl}[1,2,4]triazolo[4,3-a]pyrimidin-7 (8H)-one (77);
3-[(2-fluorobenzyl)sulfanyl]-5,6-dimethyl[1,2,4]triazolo[4,3-a]pyrimidin-7 (8H)-one (78);
3-[(3-chlorobenzyl)sulfanyl]-5,6-dimethyl[1,2,4]triazolo[4,3-a]pyrimidin-7 (8H)-one (79);
3-[(2,4-dichlorobenzyl)sulfanyl]-5,6-dimethyl[1,2,4]triazolo[4,3-a]pyrimidin-7 (8H)-one (80);
3-[(3-chlorobenzyl)sulfanyl]-5-methyl[1,2,4]triazolo[4,3-a]pyrimidin-7 (8H)-one (81);
3-[(3-fluorobenzyl)sulfanyl]-5,6-dimethyl[1,2,4]triazolo[4,3-a]pyrimidin-7 (8H)-one (82);
3-[(2-chloro-6-fluorobenzyl)sulfanyl]-5,6-dimethyl[1,2,4]triazolo[4,3-a]pyrimidin-7 (8H)-one (83);
3-[(4-bromobenzyl)sulfanyl]-5,6-dimethyl[1,2,4]triazolo[4,3-a]pyrimidin-7 (8H)-one (84);
3-[(4-chlorobenzyl)sulfanyl]-5-methyl[1,2,4]triazolo[4,3-a]pyrimidin-7 (8H)-one (85);
3-(benzylsulfanyl)-5-propyl[1,2,4]triazolo[4,3-a]pyrimidin-7 (8H)-one (86);
3-[(2-methylbenzyl)sulfanyl]-5-propyl[1,2,4]triazolo[4,3-a]pyrimidin-7 (8H)-one (87);
3-[(3-methylbenzyl)sulfanyl]-5-propyl[1,2,4]triazolo[4,3-a]pyrimidin-7 (8H)-one (88);
3-[(4-methylbenzyl)sulfanyl]-5-propyl[1,2,4]triazolo[4,3-a]pyrimidin-7 (8H)-one (89);
3-[(naphthalen-1-ylmethyl)sulfanyl]-5-propyl[1,2,4]triazolo[4,3-a]pyrimidin-7 (8H)-one (90);
3-[(3-fluorobenzyl)sulfanyl]-5-propyl[1,2,4]triazolo[4,3-a]pyrimidin-7 (8H)-one (91);
3-[(4-fluorobenzyl)sulfanyl]-5-propyl[1,2,4]triazolo[4,3-a]pyrimidin-7 (8H)-one (92);
3-[(2-chlorobenzyl)sulfanyl]-5-propyl[1,2,4]triazolo[4,3-a]pyrimidin-7 (8H)-one (93);
3-[(3,4-dichlorobenzyl)sulfanyl]-5-propyl[1,2,4]triazolo[4,3-a]pyrimidin-7 (8H)-one (94);
3-[(2-fluorobenzyl)sulfanyl]-5-propyl[1,2,4]triazolo[4,3-a]pyrimidin-7 (8H)-one (95);
3-[(2,4-dichlorobenzyl)sulfanyl]-5-propyl[1,2,4]triazolo[4,3-a]pyrimidin-7 (8H)-one (96);
3-[(3-fluorobenzyl)sulfanyl]-5-methyl[1,2,4]triazolo[4,3-a]pyrimidin-7 (8H)-one (97);
3-[(2-fluorobenzyl)sulfanyl]-5-methyl[1,2,4]triazolo[4,3-a]pyrimidin-7 (8H)-one (98);
3-[(2,5-dimethylbenzyl)sulfanyl]-5-methyl[1,2,4]triazolo[4,3-a]pyrimidin-7 (8H)-one and (99);
3-[(3-chlorobenzyl)sulfanyl]-5-propyl[1,2,4]triazolo[4,3-a]pyrimidin-7 (8H)-one (100).

In another further particular embodiment, is provided a compound for use as a medicament wherein said compound is selected from compounds (1) to (100) as defined herein.

In another further particular embodiment, is provided a compound of the invention selected from the following group:
3-[(3,4-difluorobenzyl)sulfanyl]-5-propyl[1,2,4]triazolo[4,3-a]pyrimidin-7 (8H)-one (1);
3-[(2-chloro-5-fluorobenzyl)sulfanyl]-5-propyl[1,2,4]triazolo[4,3-a]pyrimidin-7 (8H)-one (2);
3-[(3,5-difluorobenzyl)sulfanyl]-5-propyl[1,2,4]triazolo[4,3-a]pyrimidin-7 (8H)-one (3);
3-{[3-(difluoromethyl)benzyl]sulfanyl}-5-propyl[1,2,4]triazolo[4,3-a]pyrimidin-7 (8H)-one (4);
3-{[2-(difluoromethyl)benzyl]sulfanyl}-5-propyl[1,2,4]triazolo[4,3-a]pyrimidin-7 (8H)-one (5);
3-{[4-(difluoromethyl)benzyl]sulfanyl}-5-propyl[1,2,4]triazolo[4,3-a]pyrimidin-7 (8H)-one (6);
3-{[(7-oxo-5-propyl-7,8-dihydro[1,2,4]triazolo[4,3-a]pyrimidin-3-yl)sulfanyl]methyl}benzonitrile (7);
4-fluoro-2-{[(7-oxo-5-propyl-7,8-dihydro[1,2,4]triazolo[4,3-a]pyrimidin-3-yl)sulfanyl]methyl}benzonitrile (8);
3-[(5-fluoro-2-methoxybenzyl)sulfanyl]-5-propyl[1,2,4]triazolo[4,3-a]pyrimidin-7 (8H)-one (9);
3-[(3-chloro-5-fluorobenzyl)sulfanyl]-5-propyl[1,2,4]triazolo[4,3-a]pyrimidin-7 (8H)-one (10);
3-[(4-chloro-3-fluorobenzyl)sulfanyl]-5-propyl[1,2,4]triazolo[4,3-a]pyrimidin-7 (8H)-one (11);
3-[(5-fluoro-2-methylbenzyl)sulfanyl]-5-propyl[1,2,4]triazolo[4,3-a]pyrimidin-7 (8H)-one (12);
3-[(4-fluoro-2-methylbenzyl)sulfanyl]-5-propyl[1,2,4]triazolo[4,3-a]pyrimidin-7 (8H)-one (13);
3-[(2-phenylethyl)sulfanyl]-5-propyl[1,2,4]triazolo[4,3-a]pyrimidin-7 (8H)-one (14);
3-[(furan-3-ylmethyl)sulfanyl]-5-propyl[1,2,4]triazolo[4,3-a]pyrimidin-7 (8H)-one (15);
3-[(2-methoxybenzyl)sulfanyl]-5-propyl[1,2,4]triazolo[4,3-a]pyrimidin-7 (8H)-one (16);
3-[(3-methoxybenzyl)sulfanyl]-5-propyl[1,2,4]triazolo[4,3-a]pyrimidin-7 (8H)-one (17);
4-{[(7-oxo-5-propyl-7,8-dihydro[1,2,4]triazolo[4,3-a]pyrimidin-3-yl)sulfanyl]methyl}benzonitrile (18);
3-[(1-benzofuran-5-ylmethyl)sulfanyl]-5-propyl[1,2,4]triazolo[4,3-a]pyrimidin-7 (8H)-one (19);
3-[(4-methoxybenzyl)sulfanyl]-5-propyl[1,2,4]triazolo[4,3-a]pyrimidin-7 (8H)-one (20);
3-[(2,5-difluorobenzyl)sulfanyl]-5-propyl[1,2,4]triazolo[4,3-a]pyrimidin-7 (8H)-one (21);
3-[(2,4-difluorobenzyl)sulfanyl]-5-propyl[1,2,4]triazolo[4,3-a]pyrimidin-7 (8H)-one (22);
3-[(2,6-difluorobenzyl)sulfanyl]-5-propyl[1,2,4]triazolo[4,3-a]pyrimidin-7 (8H)-one (23);
5-propyl-3-[(1H-pyrazol-4-ylmethyl)sulfanyl][1,2,4]triazolo[4,3-a]pyrimidin-7 (8H)-one (24);
3-[(2,6-dimethylbenzyl)sulfanyl]-5-propyl[1,2,4]triazolo[4,3-a]pyrimidin-7 (8H)-one (25);
3-[(2-chloro-6-fluorobenzyl)sulfanyl]-5-propyl[1,2,4]triazolo[4,3-a]pyrimidin-7 (8H)-one (26);
3-[(2,5-dimethylbenzyl)sulfanyl]-5-propyl[1,2,4]triazolo[4,3-a]pyrimidin-7 (8H)-one (27);
3-{[2-(2,4-difluorophenyl)ethyl]sulfanyl}-5-propyl[1,2,4]triazolo[4,3-a]pyrimidin-7 (8H)-one (28);
3-[(2,5-difluorobenzyl)sulfanyl]-5-methyl[1,2,4]triazolo[4,3-a]pyrimidin-7 (8H)-one (29);
5-[2-(benzyloxy)ethyl]-3-[(2,5-difluorobenzyl)sulfanyl][1,2,4]triazolo[4,3-a]pyrimidin-7 (8H)-one (30);
3-[(2,5-difluorobenzyl)sulfanyl]-5-(methoxymethyl) [1,2,4]triazolo[4,3-a]pyrimidin-7 (8H)-one (31);
5-{2-[(4-chlorobenzyl)oxy]ethyl}-3-[(2,5-difluorobenzyl)sulfanyl][1,2,4]triazolo[4,3-a]pyrimidin-7 (8H)-one (32);
5-{2-[(2-chlorobenzyl)oxy]ethyl}-3-[(2,5-difluorobenzyl)sulfanyl][1,2,4]triazolo[4,3-a]pyrimidin-7 (8H)-one (33);
5-{2-[(3-chlorobenzyl)oxy]ethyl}-3-[(2,5-difluorobenzyl)sulfanyl][1,2,4]triazolo[4,3-a]pyrimidin-7 (8H)-one (34);
3-[(2,5-difluorobenzyl)sulfanyl]-5-(2-methoxyethyl) [1,2,4]triazolo[4,3-a]pyrimidin-7 (8H)-one (35);
3-[(2-chloro-6-fluorobenzyl)sulfanyl]-5-methyl[1,2,4]triazolo[4,3-a]pyrimidin-7 (8H)-one (36);
3-[(2-chloro-6-fluorobenzyl)sulfanyl]-5-ethyl[1,2,4]triazolo[4,3-a]pyrimidin-7 (8H)-one (37);
5-butyl-3-[(2-chloro-6-fluorobenzyl)sulfanyl][1,2,4]triazolo[4,3-a]pyrimidin-7 (8H)-one (38);
3-[(2-chloro-6-fluorobenzyl)sulfanyl]-5-cyclopropyl[1,2,4]triazolo[4,3-a]pyrimidin-7 (8H)-one (39);
3-[(2-chloro-6-fluorobenzyl)sulfanyl]-5-(2-methylpropyl) [1,2,4]triazolo[4,3-a]pyrimidin-7 (8H)-one (40);
3-[(2-chloro-6-fluorobenzyl)sulfanyl]-5-(cyclopropylmethyl) [1,2,4]triazolo[4,3-a]pyrimidin-7 (8H)-one (41);
3-[(2-chloro-6-fluorobenzyl)sulfanyl]-5-(methoxymethyl) [1,2,4]triazolo[4,3-a]pyrimidin-7 (8H)-one (42);
5-[2-(benzyloxy)ethyl]-3-[(2-chloro-6-fluorobenzyl)sulfanyl][1,2,4]triazolo[4,3-a]pyrimidin-7 (1H)-one (43);
3-[(2-chloro-6-fluorobenzyl)sulfanyl]-5-(3,3,3-trifluoropropyl) [1,2,4]triazolo[4,3-a]pyrimidin-7 (8H)-one (44);
5-benzyl-3-[(2-chloro-6-fluorobenzyl)sulfanyl][1,2,4]triazolo[4,3-a]pyrimidin-7 (8H)-one (45);
4-{[(5-benzyl-7-oxo-7,8-dihydro[1,2,4]triazolo[4,3-a]pyrimidin-3-yl)sulfanyl]methyl}benzonitrile (46);
3-(phenylsulfanyl)-5-propyl[1,2,4]triazolo[4,3-a]pyrimidin-7 (8H)-one (47);
3-[(2-chloro-6-fluorobenzyl)sulfanyl]-6-methyl-5-propyl[1,2,4]triazolo[4,3-a]pyrimidin-7 (8H)-one (48);
3-[(2-chloro-6-fluorobenzyl)amino]-5-propyl[1,2,4]triazolo[4,3-a]pyrimidin-7 (8H)-one (49);
3-[(2-chloro-6-fluorobenzyl)sulfonyl]-5-propyl[1,2,4]triazolo[4,3-a]pyrimidin-7 (8H)-one (50); and
3-(2-phenylethyl)-5-propyl[1,2,4]triazolo[4,3-a]pyrimidin-7 (8H)-one (51).

According to a particular aspect, is provided a compound of the invention for use in combination with immunotherapy.

According to a particular aspect, is provided a compound of the invention for use in combination with T-cell Transfer therapies such as chimeric antigen receptor (CAR) T-cells, tumour infiltrating lymphocytes (TILs) or T cell receptor (TCR) therapies.

According to a further particular aspect, is provided a compound of the invention for use in combination with a cancer vaccine or with at least one immune checkpoint inhibitor.

According to a particular aspect, is provided a compound of the invention for use in combination with an immune checkpoint inhibitor.

According to a particular aspect, an immune checkpoint inhibitor is selected from a PD1 inhibitor, PD-L1 inhibitor, CTLA-4 inhibitor, VISTA inhibitor, CD155/TIGIT inhibitor, TIM-3 inhibitor.

According to a particular aspect, an immune checkpoint inhibitor according to the invention is a PD-1 inhibitor.

According to a particular aspect, is provided a compound of the invention for use in combination with an immunotherapeutic agent, while said immunotherapeutic agent is at least one monoclonal antibody such as rituximab and blinatumomab.

According to a particular aspect, is provided a compound of the invention for use in combination with an immunotherapeutic agent, wherein said immunotherapeutic agent is at least one cytokine such as interferon and aldesleukin.

According to another further particular aspect, is provided a compound of the invention for use in combination with an anti-cancer vaccine.

According to a particular aspect, an anti-cancer vaccine is selected from a DNA, RNA, peptide and a oncolytic virus vaccine.

According to a particular aspect, is provided a compound of the invention for use in combination with a cancer vaccine such as an oncolytic or anti Herpes simplex vaccine.

According to another further particular aspect, is provided a compound of the invention for use for the activation of both humoral and cellular immunity, a prerequisite for successful virus neutralization and/or elimination (such as SARS-COV-2).

According to a further aspect, is provided a compound of the invention for use as a vaccine adjuvant component, complementary to a classic adjuvant, to improve the longevity of both the cellular and humoral immune response upon vaccination.

According to another further particular aspect, is provided a compound of the invention for use in combination with chemotherapies such as alkylating agents, nitrosoureas, anti-metabolites, plant alkaloids and natural products, anti-tumor antibiotics, hormonal agents, biological response modifiers, depending on cancer type.

According to another further particular aspect, is provided a compound of the invention for use in combination with radiotherapies including intensity-modulated radiation therapy (IMRT), Volumetric modulated radiation therapy (VMAT) and Image-guided radiation therapy (IGRT).

### Synthesis of Compounds According to the Invention

Compounds of the invention can be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

The general synthetic approaches for obtaining compounds of Formula (I) is depicted in Schemes 1 and 2 below.

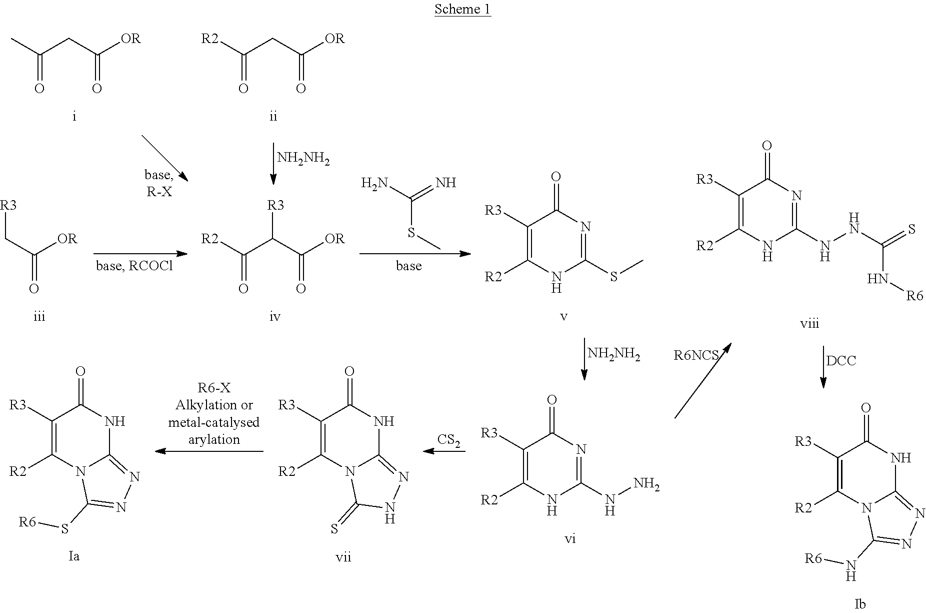

R2 and R3-functionalized β-ketoacetates (iv) can be accessed via a wide range of transformations from readily available starting materials, including but not limited to base catalyzed alkylation or electrophilic substitution of simple acetylacetates (i) or β-ketoacetates unsubstituted at the alpha position (ii), or via acetylation of acetates (iii). Base catalyzed condensation with S-methylisothiourea leads to intermediate 2-methylsulfanyl-1H-pyrimidin-4-ones (v), which undergo nucleophilic aromatic substitiion with hydrazine to give 2-hydrazino-1H-pyrimidin-4-ones (vi). Intermediates (vi) can undergo cyclization with carbondisulfide to 3-thioxo-2,8-dihydro-[1,2,4]triazolo[4,3-a]pyrimidin-7-ones (vii) which can then undergo a variety of transformations, including but not limited to base-mediated S-alkylation or metal-mediated (e.g. via copper-iodide)S-arylation to give final compounds of Formula (I), in particular of Formula (Ia). In case of preparation of isotopic isomers, isotopically labelled building blocks can be used in Scheme 1 such as C13 or C14 Carbon disulphide, N15 hydrazine etc.

Intermediate (vi) can undergo reaction with isothiocyanates to yield aminothioureas (viii) which in turn cyclize in the presence of DCC or similar peptide-coupling agents to yield final compound of Formula (I), in particular of Formula (Ib).

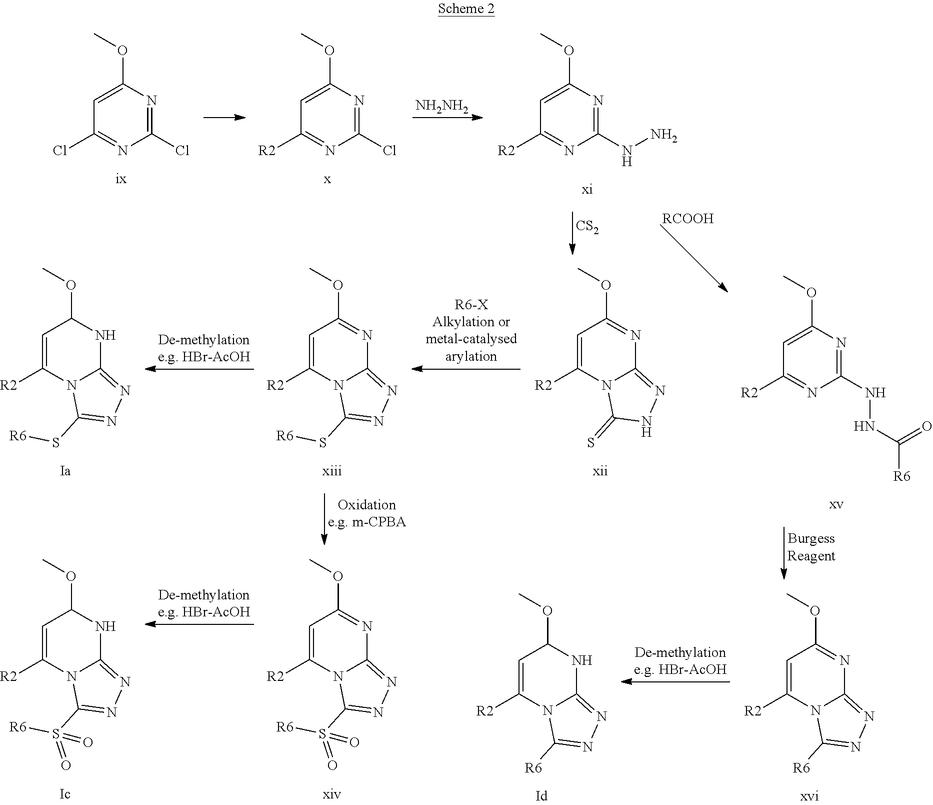

2,4-dichloro-6-methoxypyrimidine (ix) undergoes regioselective alkylation with metal alkyls such as Grignard reagents to yield functionalized 2-chloro-6-methoxyparimidines (x), which in turn can undergo nucleophilic aromatic substitution with hydrazine to give 2-hydrazino-6-methoxy-pyrimidine intermediates (xi). Cyclization of (xi) with carbondisulfide gives 7-methoxy-2H-[1,2,4]triazolo[4,3-a]pyrimidine-3-thiones (xii), which can undergo further functionalization on the thiogroup via alkylation or metal mediated arylation to give 7-methoxy-3-sulfanyl-[1,2,4]triazolo[4,3-a]pyrimidines (xiii) substituted at the 3 position. Intermediates (xiii) can undergo acid-mediated demethylation at the 7 position e.g. via hydrobromic acid to give final compounds of Formula (I), in particular of Formula (Ia); alternatively compounds (xiii) can undergo further functionalization such as meta-chloroperoxybenzoic acid (m-CPBA) mediated oxidation to 7-methoxy-3-sulfonyl-[1,2,4]triazolo[4,3-a]pyrimidines (xiv), which can then in turn undergo acid-mediated demethylation at the 7-position to give final compounds of Formula (I), in particular of Formula (Ic).

Intermediate (xi) can form acetohydride intermediates (xv) via reaction with carboxylic acids under amide coupling conditions (e.g. EDC/HOBt), which can then by cyclized via dehydration mediated via e.g. Burgess reagent to 7-methoxy-3-alkyl-[1,2,4]triazolo[4,3-a]pyrimidine or 7-methoxy-3-aryl-[1,2,4]triazolo[4,3-a]pyrimidine intermediates (xvi), which can then in turn undergo acid mediated demethylation at the 7-position to give final compounds of Formula (I) in particular of Formula (Id).

Further functionalization or functional group manipulation of sidechains in the final compounds or intermediates can be realized by those skilled in the art, broadening the scope of final compounds of Formula (I) accessible via scheme 1 and scheme 2.

### Compositions

The invention provides pharmaceutical or therapeutic agents as compositions and methods for treating a patient, preferably a mammalian patient, and most preferably a human patient who is suffering from a solid tumor cancer presenting or susceptible to present a resistance to immunotherapy.

Pharmaceutical compositions of the invention can contain one or more compound in any form described herein. Compositions of this invention may further comprise one or more pharmaceutically acceptable additional ingredient(s), such as alum, solubilizers, stabilizers, antimicrobial agents, buffers, coloring agents, flavoring agents, adjuvants, and the like.

The compounds of the invention, together with a conventionally employed adjuvant, carrier, diluent or excipient may be placed into the form of pharmaceutical compositions and unit dosages thereof, and in such form may be employed as solids, such as powder in sachets, tablets or filled capsules, or liquids such as solutions, suspensions, emulsions, elixirs, nasal spray, or capsules filled with the same, all for oral use, or in the form of sterile injectable solutions for parenteral (including subcutaneous) use. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed. Compositions according to the invention are preferably oral, sublingual, nasal and subcutaneous.

Compositions of this invention may also be liquid formulations, including, but not limited to, aqueous or oily suspensions, solutions, emulsions, syrups, spray and elixirs. Liquid forms suitable for oral administration may include a suitable aqueous or non-aqueous vehicle with buffers, suspending and dispensing agents, colorants, flavors and the like. The compositions may also be formulated as a dry product for reconstitution with water or other suitable vehicle before use. Such liquid preparations may contain additives, including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Non aqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid. Further materials as well as processing techniques and the like are set out in Remington: The Science & Practice of Pharmacy, 23^{rd} Edition, 2020, Ed. Adeboye Adejare, Academic Press, which is incorporated herein by reference.

Solid compositions of this invention may be in the form of powder in sachets, tablets or lozenges formulated in a conventional manner. For example, sachets, tablets and capsules for oral or sublingual administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

Injectable compositions are typically based upon injectable sterile saline or phosphate-buffered saline or other injectable carriers known in the art.

Compositions of this invention may also be formulated for parenteral administration, including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions of this invention may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

The compounds of this invention can also be administered in sustained release forms or from sustained release drug delivery systems. A description of representative sustained release materials can also be found in the incorporated materials in Remington's Pharmaceutical Sciences.

### Mode of Administration

Compositions of this invention may be administered in any manner, including, but not limited to, topically, orally, parenterally, sublingually, via buccal administration, nasally, intralesionally, in cerebral ventriculesor combinations thereof. Parenteral administration includes, but is not limited to subcutaneous and intramuscular. The compositions of this invention may also be administered in the form of an implant, which allows slow release of the compositions as well as a slow controlled i.v. infusion. In a particular embodiment, one or more compound of the invention is administered orally.

The dosage administered, as single or multiple doses, to an individual will vary depending upon a variety of factors, including pharmacokinetic properties, patient conditions and characteristics (age, body weight, health, body size), extent of symptoms, frequency of treatment and the effect desired.

### Combination

According to one embodiment of the invention, a compound according to the invention and pharmaceutical formulations thereof is to be administered or used in combination with an anti-cancer immunotherapeutic agent, in particular an anticancer vaccine or at least one immune check point inhibitor such as at least one PD-1, PD-L1 or CTLA4 inhibitor.

The invention encompasses the administration of a compound of the invention or a pharmaceutical formulation thereof, wherein said compound of the invention or a pharmaceutical formulation thereof is administered to an individual prior to, or simultaneously with an anti-cancer immunotherapeutic agent, for example concomitantly through the same formulation or separately through different formulations, in particular through different formulation routes.

According to a particular aspect of the invention, a compound according to the invention and pharmaceutical formulations thereof is to be administered chronically (e.g. daily or weekly) for the duration of treatment and prior to the administration of an anti-cancer immunotherapeutic agent or the anti-angiogenic treatment.

According to another particular aspect of the invention, a compound according to the invention and pharmaceutical formulations thereof is to be administered concomitantly with an anti-cancer immunotherapeutic agent.

According to another particular aspect of the invention, the anti-cancer immunotherapeutic agent can be administered in combination with other therapeutic regimens or co-agents useful in the treatment of cancer (e.g. multiple drug regimens), in a therapeutically effective amount, such as in combination with substances useful for treating, stabilizing, preventing, and/or delaying cancer such as substances used in conventional chemotherapy directed against solid tumors and for control of establishment of metastases or any other molecule that act by triggering programmed cell death. In particular, according to another particular aspect of the invention, the anti-cancer immunotherapeutic agent can be administered in combination with other therapeutic regimens or co-agents useful in the treatment of cancer (e.g. multiple drug regimens), in a therapeutically effective amount.

Compounds of the invention or the pharmaceutical formulations thereof that are administered simultaneously with said anti-cancer immunotherapeutic agent can be administered in or within the same or different composition(s) and by the same or different route(s) of administration.

### Patients

In one embodiment, subjects according to the invention are subjects suffering from a solid tumor cancer, in particular a poorly responsive solid tumor cancer presenting or susceptible to present a resistance to immunotherapy.

In a particular embodiment, subjects according to the invention are subjects suffering from a solid tumor cancer selected from lung cancer (small cell and non-small cell), breast cancer, ovarian cancer, cervical cancer, uterus cancer, head and neck cancer, melanoma, hepatocellular carcinoma, colon cancer, rectal cancer, colorectal carcinoma, kidney cancer, prostate cancer, gastric cancer, bronchus cancer, pancreatic cancer, urinary bladder cancer, hepatic cancer and brain cancer, in particular glioblastoma.

In another particular embodiment, subjects according to the invention are subjects suffering from head and neck tumors.

In another particular embodiment, subjects according to the invention are subjects suffering from melanoma.

In another particular embodiment, subjects according to the invention are subjects suffering from colon cancer.

In another particular embodiment, subjects according to the invention are subjects suffering from lung carcinoma.

In another particular embodiment, subjects according to the invention are subjects suffering from breast cancer.

In another particular embodiment, subjects according to the invention are subjects suffering from hepatocellular carcinoma or hepatic cancer.

In another particular embodiment, subjects according to the invention are subjects suffering from rectal cancer or colorectal carcinoma.

In another particular embodiment, subjects according to the invention are subjects suffering from kidney cancer.

In another particular embodiment, subjects according to the invention are subjects suffering from pancreatic cancer.

In another particular embodiment, subjects according to the invention are subjects suffering from brain cancer, in particular glioblastoma.

In another particular embodiment, subjects according to the invention are subjects with solid tumor cancer who are at risk of developing resistance or partial resistance to anti-cancer immunotherapy due to another concomitant treatment or a genetic pre-disposition.

In another particular embodiment, subjects according to the invention are subjects suffering from a metabolic diseases such as type 2 diabetes.

In another particular embodiment, subjects according to the invention are subjects suffering from a fibrotic disease such as pulmonary fibrosis or non alcoholic steatohepatitis (NASH).

In another particular embodiment, subjects according to the invention are subjects suffering from an auto-immune disease such as multiple sclerosis.

In another particular embodiment, subjects according to the invention are subjects suffering from an hair loss disorder such as alopecia.

In another particular embodiment, subjects according to the invention are subjects suffering from a neurodegenerative disorder such as Parkinson or Alzheimer's disease.

In another particular embodiment, subjects according to the invention are subjects suffering from a skin or tissue injury such a skin wound or a burn.

In another particular embodiment, subjects according to the invention are subjects suffering from an acute pathology of the brain such as stroke or brain trauma.

### Use According to the Invention

In a particular embodiment, the invention provides compounds, methods, uses and compositions useful for the prevention and/or treatment of a hair loss disorders.

In another particular embodiment, the invention provides compounds, methods, uses and compositions useful for the prevention and/or treatment of a skin or tissue injury such a skin wound or a burn. Typically, the compounds of the invention are to be topically applied to the skin or tissue.

Typically, according to a particular embodiment, the compounds of the invention are to be topically applied to the skin.

In another particular embodiment, the invention provides compounds, methods, uses and compositions useful for the prevention and/or treatment of an auto-immune disease such as multiple sclerosis.

In another particular embodiment, the invention provides compounds, methods, uses and compositions useful for the prevention and/or treatment of a neurodegenerative disorder such as Parkinson or Alzheimer's disease.

In another particular embodiment, the invention provides compounds, methods, uses and compositions useful for the prevention and/or treatment of a fibrotic disease such as such as pulmonary fibrosis or non alcoholic steatohepatitis (NASH).

In another particular embodiment, the invention provides compounds, methods, uses and compositions useful for the prevention and/or treatment of an acute pathology of the brain such as stroke or brain trauma.

In another particular embodiment, the invention provides compounds, methods, uses and compositions useful for the prevention and/or treatment of a metabolic diseases such as type 2 diabetes.

Typically, according to a particular embodiment, the compounds of the invention are to be administered orally.

According to a particular aspect, is provided for eliciting or increasing an immune response to immunotherapy, in particular anti-cancer immunotherapy, or to vaccinotherapy, said method comprising administering an effective amount of one or more compound of the invention or a pharmaceutical formulation thereof in combination with an immunotherapeutic agent in a subject in need thereof.

In a particular embodiment, the invention provides compounds, methods, uses and compositions are useful for the treatment of a solid tumor cancer in the form of a combination wherein at least one compound of the invention is to be administered in combination with a vaccine, in particular an anti-cancer vaccine such as an oncolytic vaccine or anti-Herpes simplex virus vaccines.

According to a particular aspect, is provided a method for treating a subject suffering from a cancer, said method comprising administering an effective amount of one or more compound of the invention in combination with an anti-cancer immunotherapeutic agent in a subject in need thereof.

In a particular embodiment, the invention provides compounds, methods, uses and compositions are useful for the treatment of a solid tumor cancer in the form of a combination wherein at least one compound of the invention is to be administered in combination with at least one anti-cancer immunotherapeutic agent.

According to a particular aspect, is provided a method for treating a subject suffering from a cancer, said method comprising administering an effective amount of one or more compound of the invention in combination with an anti-cancer immunotherapeutic agent in a subject in need thereof.

According to a particular aspect, is provided an in vitro method for obtaining and/or maintaining T-cells with a memory phenotype, said method comprising the following steps:
Providing at least one T-cell for which has the ability to differentiate into a memory phenotype, such as CD8+ or CD4+ T cell;
Contacting said at least one T-cell with at least one compound according to the invention or a mixture thereof;
CuIturing the cells in a T-cell culture medium;
Isolating the obtained T-cells.

According to a particular embodiment, the said at least one compound from the invention is comprised in the medium used for culturing the T-cells and the T-cells are then cultured in the presence of the said at least one compound of the invention.

In another embodiment, the said at least one compound of the invention is added to the culture after the T-cell culture is initiated, e.g. after or during the cells are seeded or incubated, but ideally shortly after culture starts.

According to a particular aspect, the T-cells are contacted with the compound of the invention at least during activation, preferably from the beginning of the culture and/or activation, for example for the first 3 or 4 days.

According to another particular aspect, the T-cells may be contacted with the compound of the invention during the entire culture period.

According to another particular aspect, the compound of the invention is washed away after the initial activation phase (priming phase), and the culture is continued in absence of the compound of the invention, e.g. with a medium comprising IL-2 and IL-7.

It is preferred that the compound of the invention is present in the culture medium from the beginning of the culture. Moreover, preferably, the T-cells are at least contacted with the compound of the invention, although it is not strictly necessary that the compound of the invention is present during the entire activation phase. Hence, the compounds of the invention are used for culturing T-cells, as described herein, in particular for generating and/or maintaining T-cells with a memory phenotype.

Activated T-cells according to the invention, may then be used to improve commonly practiced anti-cancer T-cell immunotherapies.

References cited herein are hereby incorporated by reference in their entirety. The invention having been described, the following examples are presented by way of illustration, and not limitation.

### EXAMPLES

Several methods for preparing the compounds of this invention are illustrated in the following Examples. Unless otherwise noted, all starting materials were obtained from commercial suppliers and used without further purification. Specifically, the following abbreviations may be used in the examples and throughout the specification.

The following abbreviations refer respectively to the definitions below:
AMU (Atomic Mass Unit); CDI (Carbonyldiimidazole); DCC (dicyclohexyl carbodiimide); DCM (dichloromethane); DIPEA (Di-isopropylethylamine); DMF (Dimethylformamide); DMSO (Dimethyl Sulfoxide); EDC (1-[3-(Dimethylamino) propyl]-3-ethylcarbodiimide methiodide); HOBt (1-hydroxybenotriazole); IPA (Isopropyl alcohol); HMBC (Heteronuclear Multiple Bond Correlation); m-CPBA (meta-chloroperoxybenzoic acid); TEA (Triethylamine).

### Example 1: Synthesis of Compounds of the Invention

Compounds of the invention have been synthesized as described herein.

### a) Compounds of Formula (I) Wherein R7 is an Optionally Substituted Phenyl

Compounds 1-28 were synthesized according to Scheme 1 from the description.

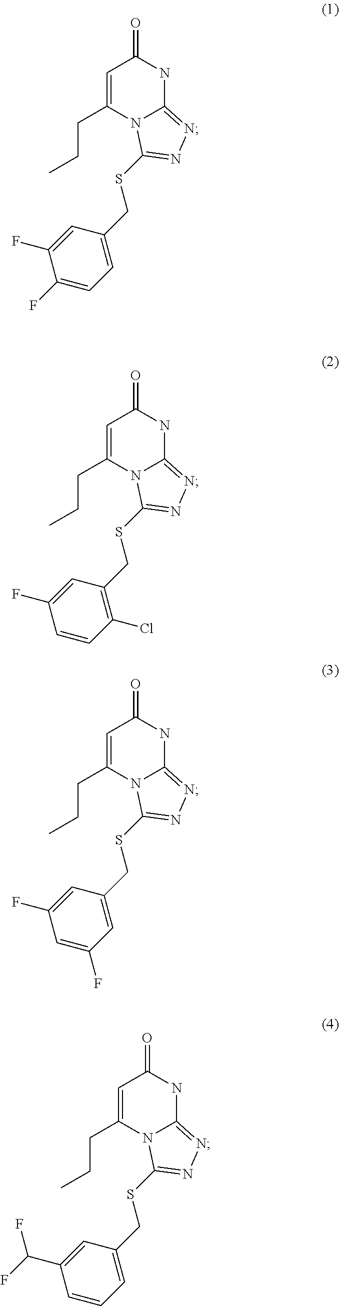 [SMILES: CCCc1cc(=O)[nH]c2nnc(SCc3ccc(F)c(F)c3)n12 ; CCCc1cc(=O)[nH]c2nnc(SCc3cc(F)ccc3Cl)n12 ; CCCc1cc(=O)[nH]c2nnc(SCc3cc(F)cc(F)c3)n12 ; CCCc1cc(=O)[nH]c2nnc(SCc3cccc(C(F)F)c3)n12]

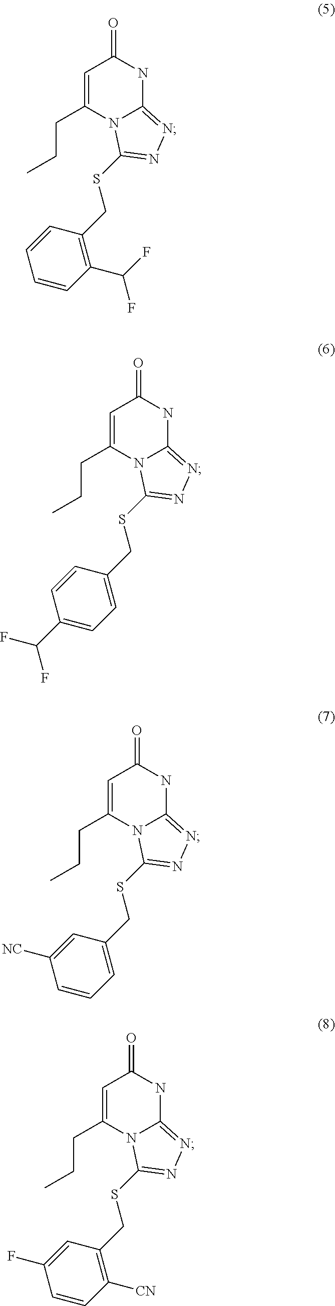 [SMILES: CCCc1cc(=O)[nH]c2nnc(SCc3ccccc3C(F)F)n12 ; CCCc1cc(=O)[nH]c2nnc(SCc3ccc(C(F)F)cc3)n12 ; CCCc1cc(=O)[nH]c2nnc(SCc3cccc(C#N)c3)n12 ; CCCc1cc(=O)[nH]c2nnc(SCc3cc(F)ccc3C#N)n12]

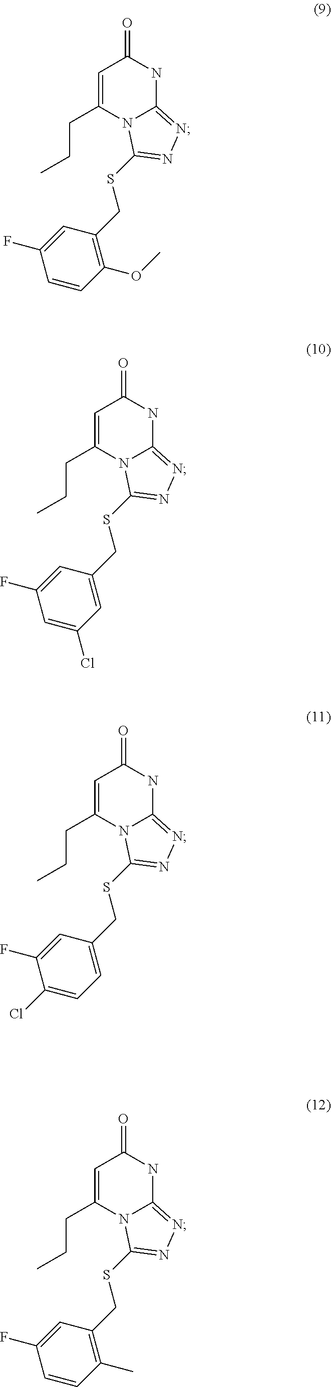 [SMILES: CCCc1cc(=O)[nH]c2nnc(SCc3cc(F)ccc3OC)n12 ; CCCc1cc(=O)[nH]c2nnc(SCc3cc(F)cc(Cl)c3)n12 ; CCCc1cc(=O)[nH]c2nnc(SCc3ccc(Cl)c(F)c3)n12 ; CCCc1cc(=O)[nH]c2nnc(SCc3cc(F)ccc3C)n12]

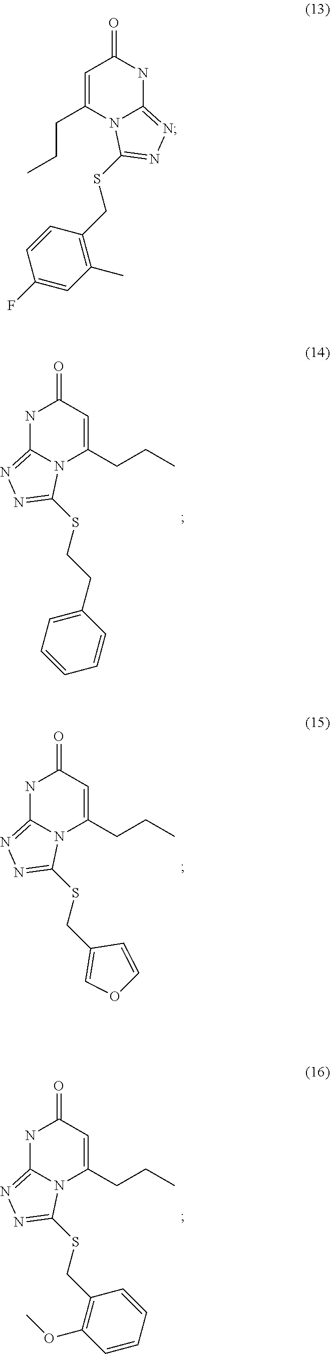 [SMILES: CCCc1cc(=O)[nH]c2nnc(SCc3ccc(F)cc3C)n12 ; CCCc1cc(=O)[nH]c2nnc(SCCc3ccccc3)n12 ; CCCc1cc(=O)[nH]c2nnc(SCc3ccoc3)n12 ; CCCc1cc(=O)[nH]c2nnc(SCc3ccccc3OC)n12]

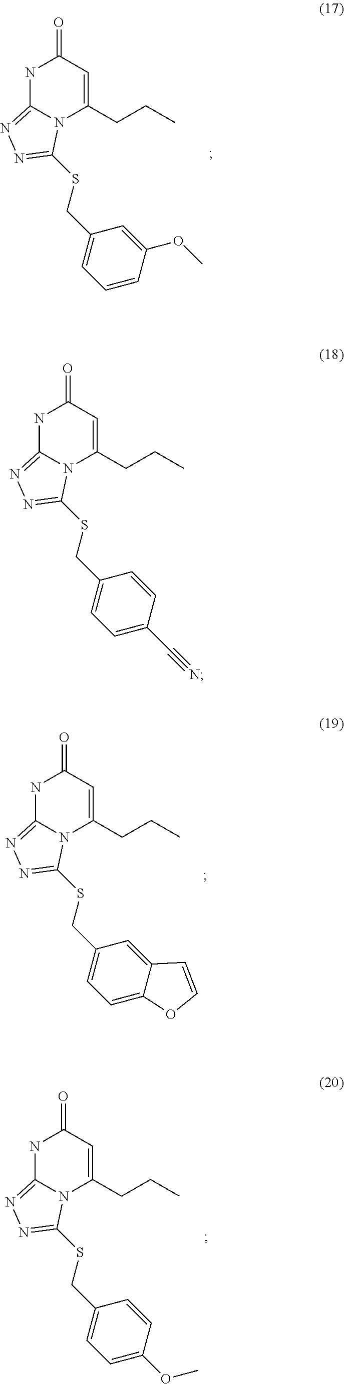 [SMILES: CCCc1cc(=O)[nH]c2nnc(SCc3cccc(OC)c3)n12 ; CCCc1cc(=O)[nH]c2nnc(SCc3ccc(C#N)cc3)n12 ; CCCc1cc(=O)[nH]c2nnc(SCc3ccc4occc4c3)n12 ; CCCc1cc(=O)[nH]c2nnc(SCc3ccc(OC)cc3)n12]

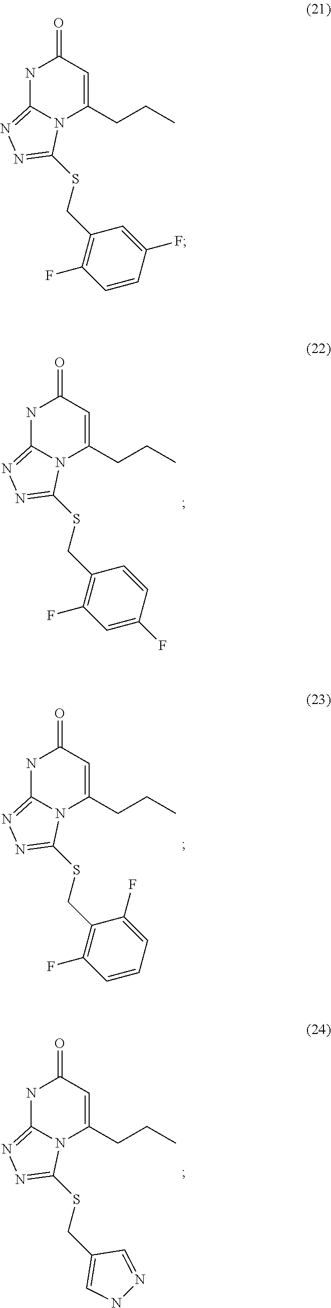 [SMILES: CCCc1cc(=O)[nH]c2nnc(SCc3cc(F)ccc3F)n12 ; CCCc1cc(=O)[nH]c2nnc(SCc3ccc(F)cc3F)n12 ; CCCc1cc(=O)[nH]c2nnc(SCc3c(F)cccc3F)n12 ; CCCc1cc(=O)[nH]c2nnc(SCc3cn[nH]c3)n12]

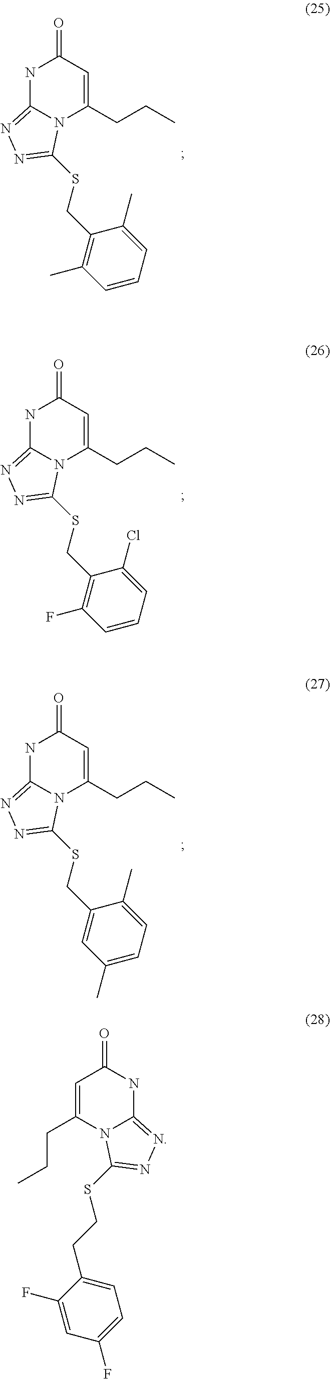

In particular, Scheme 3 below was followed wherein R is selected from a phenyl or benzyl group substituted with one or more halogen (fluoro, chloro), cyano, halogeno C₁-C₆ alkyl, C₁-C₆ alkyl; or from heteroaromatic groups such as benzofuran, furan, pyrazole.

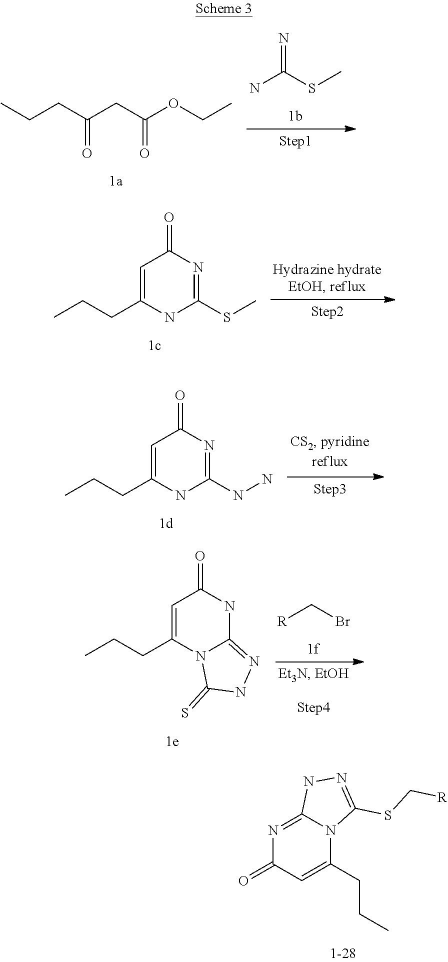

### Scheme 3, Step 1: Preparation of Intermediate (1c) (2-(methylthio)-6-propylpyrimidin-4 (1H)-one)

To a stirring solution of ethyl butyryacetate (1a) (2.5 g, 15.803 mmol) and 2-methyl-2-thiopseudourea hemisulphate (1b) (2.42 g, 17.38 mmol) in water (20 mL), was added Na₂CO₃ (2.68 g, 25.28 mmol) and stirred at room temperature for 48 h. A white precipitate was formed, which was filtered and triturated with ether to afford desired intermediate (1c) as white solid in 48.08% yield, 1.4 g. 1H NMR (400 MHz, DMSO) δ: 0.88 (t, 3H), 1.52-1.65 (m, 2H), 2.37 (t, 2H), 2.46 (s, 3H), 5.91 (s, 1H), 12.47 (brs, 1H); LCMS (Method-1): Rₜ=2.47 min; [M+H]=185 (5 min run time).

### Scheme 3, Step 2: Preparation of Intermediate (1d)

To a stirred solution of (1c) (1.4 g, 7.59 mmole) and Hydrazine hydrate (3.79 g, 75.98 mmole) in Ethanol (10 ml) was added and refluxed for 6 hours. The reaction mixture was evaporated in vacuo and triturated with EtOH to afford intermediate (1d) as white solid in 62.6% yield, 800 mg, ¹H NMR (400 MHZ, DMSO) δ: 0.86 (t, 3H), 1.50-1.59 (m, 2H), 2.22 (t, 2H), 4.48 (brs, 2H), 5.35 (s, 1H), 8.45 (brs, 1H), 9.91 (brs, 1H); LCMS (Method-1): Rₜ=1.24 min; [M+H]=169 (5 min run time).

### Scheme 3, Step 3: Preparation of Intermediate (1e)

To a stirred solution of (1d) (2 g, 11.90 mmol) in pyridine (55 mL), was added CS₂ (11 mL) and refluxed for 6 h. starting material The reaction mixture reaction mixture was evaporated in vacuo, azeotroped with toluene and finally triturated with EtOH to afford intermediate (1e) as an off white solid in 31.96% yield, 800 mg, ¹H NMR (400 MHZ, DMSO) δ: 0.95 (t, 3H), 1.56-1.73 (m, 2H), 3.36 (t, 2H), 5.82 (s, 1H), 12.54 (brs, 1H), 13.74 (brs, 1H); LCMS (Method-1): Rₜ=1.77 min; [M+H]=211 (5 min run time).

### Preparation of Compounds of the Invention (1) to (28) by Scheme 3, Step 4

To a stirred solution of intermediate (1e) (1 equiv.) and TEA (2.5 equiv.) in EtOH (20 mL/mmol), substituted benzyl bromide (1 equiv.) was added and stirred at room temperature for 18 h. The reaction mixture was evaporated in vacuo, partitioned between 10% MeOH-DCM and water. The organic phase was dried, concentrated, initially purified with Combi-flash (0.1-0.2% MeOH-DCM) and finally by prep TLC to afford the corresponding compound of the invention.

The Compounds were Characterized with the Methods Detailed Below:

Compound (1): 42 mg, White solid (26% yield). 1H NMR (400 MHZ, DMSO) δ: 0.94 (t, 3H), 1.56 (m, 2H), 2.85 (t, 2H), 4.45 (s, 2H), 5.88 (s, 1H), 7.21 (m, 1H), 7.38 (m, 1H), 7.45 (m, 1H); 12.68 (br s, 1H), LCMS (Method-2): Rₜ=2.48 min; [M+H]=353 (3 min run time) HPLC-99.60%.

Compound (2): 80 mg, White solid (32% yield). 1H NMR (400 MHZ, DMSO) δ: 0.93 (t, 3H), 1.52-1.57 (t, 2H), 2.82 (t, 2H), 4.45 (s, 2H), 5.88 (s, 1H), 7.18-7.22 (t, 1H), 7.27 (d, 1H), 7.50-7.53 (m, 1H); 12.73 (s, 1H). LCMS (Method-1): Rₜ=2.86 min; [M+H]=337 (5 min run time). HPLC=99.64%.

Compound (3): 70 mg, White solid (29% yield). 1H NMR (400 MHZ, DMSO) δ: 0.95 (t, 3H), 1.54-1.60 (t, 2H), 2.86 (t, 2H), 4.45 (s, 2H), 5.87 (s, 1H), 7.09-7.16 (t, 3H), 7.27 (d, 1H), 12.69 (s, 1H). LCMS (Method-1): Rₜ=2.37 min; [M+H]=337. HPLC=98.97%.

Compound (4): 10 5 mg, White solid (42% yield). 1H NMR (400 MHZ, DMSO) δ:0.94 (t, 3H), 1.50-1.55 (t, 2H), 2.80 (t, 2H), 4.50 (s, 2H), 5.83 (s, 1H), 6.84 (s, 1H), 6.98 (s, 1H), 7.12 (s, 1H), 7.45 (brs, 2H), 7.50 (brs, 2H), 12.68 (s, 1H). LCMS (Method-1): Rₜ=1.54 min; [M+H]=351. HPLC=98.18%.

Compound (5): 105 mg, White solid (42% yield). 1H NMR (400 MHZ, DMSO) δ:0.90 (t, 3H), 1.49-1.54 (t, 2H), 2.77 (t, 2H), 4.58 (s, 2H), 5.85 (s, 1H), 7.17-7.39 (m, 2H), 7.45 (brs, 2H), 7.60 (d, 1H), 7.45 (brs, 2H), 12.69 (s, 1H). LCMS (Method-1): Rₜ=1.56 min; [M+H]=351. HPLC=97.96%.

Compound (6): 105 mg, White solid (42% yield). 1H NMR (400 MHZ, DMSO) δ:0.92 (t, 3H), 1.49-1.57 (t, 2H), 2.82 (t, 2H), 4.50 (s, 2H), 5.85 (s, 1H), 6.99 (m, 2H), 7.49 (brs, 4H), 12.67 (s, 1H). LCMS (Method-1): Rₜ=1.55 min; [M+H]=351. HPLC-99.65%.

Compound (7): 48 mg, Off white solid (31% yield). 1H NMR (400 MHZ, DMSO) δ:0.94 (t, 3H), 1.56 (m, 2H), 2.84 (t, 2H), 4.48 (s, 2H), 5.85 (s, 1H), 7.52 (t, 1H), 7.71 (m, 2H), 7.82 (s, 1H), 12.61 (br s, 1H). LCMS (Method-5): Rₜ=2.21 min; [M+H]=326.2 (5 min run time). HPLC=99.26%.

Compound (8): 30 mg, White solid (18% yield). 1H NMR (400 MHZ, DMSO) δ:0.96 (t, 3H), 1.56-1.62 (t, 2H), 2.90 (t, 2H), 4.51 (s, 2H), 5.90 (s, 1H), 7.34-7.47 (m, 2H), 7.93 (m, 1H), 12.74 (s, 1H). LCMS (Method-1): Rₜ=1.50 min; [M+H]-344. HPLC=99.36%.

Compound (9): 60 mg, White solid (24% yield). 1H NMR (400 MHZ, DMSO) δ:0.90 (t, 3H), 1.49-1.56 (t, 2H), 2.80 (t, 2H), 3.71 (s, 3H) 4.29 (s, 2H), 5.86 (s, 1H), 6.96-7.11 (m, 3H), 12.74 (s, 1H). LCMS (Method-1): Rₜ=1.55 min; [M+H]=349. HPLC=99.64%.

Compound (10): 50 mg, White solid (20% yield). 1H NMR (400 MHZ, DMSO) δ:0.93 (t, 3H), 1.54-1.59 (t, 2H), 2.85 (t, 2H), 4.44 (s, 2H), 5.87 (s, 1H), 7.21-7.34 (m, 3H), 12.69 (s, 1H). LCMS (Method-1): Rₜ=2.48 min; [M+H]=353. HPLC=99.10%.

Compound (11): 50 mg, White solid (20% yield). 1H NMR (400 MHZ, DMSO) δ:0.92 (t, 3H), 1.53-1.59 (t, 2H), 2.85 (t, 2H), 4.45 (s, 2H), 5.86 (s, 1H), 7.21-7.54 (m, 3H), 12.68 (s, 1H). LCMS (Method-1): R=2.47 min; [M+H]=353. HPLC=99.17%.

Compound (12): 36 mg, White solid (15% yield). 1H NMR (400 MHZ, DMSO) δ:0.92 (t, 3H), 1.51-1.56 (t, 2H), 2.30 (s, 3H) 2.80 (t, 2H), 4.42 (s, 2H), 5.85 (s, 1H), 7.00-7.24 (m, 3H), 12.71 (s, 1H). LCMS (Method-1): Rₜ=1.58 min; [M+H]=333. HPLC=99.24%.

Compound (13): 60 mg, White solid (25% yield). 1H NMR (400 MHZ, DMSO) δ:0.91 (t, 3H), 1.49-1.55 (t, 2H), 2.35 (s, 3H) 2.79 (t, 2H), 4.39 (s, 2H), 5.79 (s, 1H), 6.89-7.19 (m, 3H), 12.69 (s, 1H). LCMS (Method-1): Rₜ=1.60 min; [M+H]=333. HPLC=97.64%.

Compound (14): 80 mg, White solid (27% yield). 1H NMR (400 MHZ, DMSO) &: 0.93 (t, 3H), 1.56-1.61 (m, 2H), 2.86 (t, 2H), 3.01 (t, 2H), 3.48 (t, 2H), 5.86 (s, 1H), 7.18-7.30 (m, 5H), 12.65 (brs, 1H); LCMS (Method-1): Rₜ=1.56 min; [M+H]-315 (3 min run time). HPLC-99.51%.

Compound (15): 27 mg, White solid (20% yield). 1H NMR (400 MHZ, DMSO) δ:0.95 (t, 3H), 1.56-1.62 (m, 2H), 2.88 (t, 2H), 4.30 (s, 2H), 5.87 (s, 1H), 6.44 (s, 1H), 7.59 (d, 2H), 12.66 (brs, 1H); LCMS (Method-1): Rₜ=1.48 min; [M+H]=291 (3 min run time). HPLC=97.01%.

Compound (16): 40 mg, White solid (25% yield). 1H NMR (400 MHZ, DMSO) &: 0.88 (t, 3H), 1.47-1.52 (m, 2H), 2.76 (t, 2H), 3.73 (s, 3H), 4.31 (s, 2H), 5.82 (s, 1H), 6.82 (d, 1H), 6.97 (t, 1H), 7.14 (d, 1H), 7.27 (t, 1H), 12.69 (brs, 1H); LCMS (Method-2): R1=1.60 min; [M+H]=331 (3 min run time). HPLC-96.61%.

Compound (17): 80 mg, White solid (25% yield). 1H NMR (400 MHZ, DMSO) δ:0.85 (t, 3H), 1.48-1.56 (m, 2H), 2.80 (t, 2H), 3.69 (s, 3H), 4.39 (s, 2H), 5.83 (s, 1H), 6.82-6.88 (m, 3H), 7.21 (t, 1H), 12.68 (brs, 1H); LCMS (Method-1): Rₜ=1.52 min; [M+H]=331 (3 min run time). HPLC=99.38%.

Compound (18): 36 mg, White solid (23% yield). 1H NMR (400 MHZ, DMSO) δ:0.94 (t, 3H), 1.51-1.58 (m, 2H), 2.84 (t, 2H), 4.53 (s, 2H), 5.87 (s, 1H), 7.55 (d, 2H), 7.77 (d, 2H), 12.67 (brs, 1H); LCMS (Method-2): Rₜ=1.53 min; [M+H]=326 (3 min run time). HPLC=99.56%.

Compound (19): 60 mg, White solid (25% yield) 1H NMR (400 MHZ, DMSO) δ:0.90 (t, 3H), 1.49-1.55 (m, 2H), 2.81 (t, 2H), 4.55 (s, 2H), 5.82 (s, 1H), 6.92 (s, 1H), 7.27 (d, 1H), 7.52 (d, 1H), 7.61 (s, 1H), 7.98 (d, 1H), 12.65 (brs, 1H); LCMS (Method-3): Rₜ=2.87 min; [M+H]=341 (5 min run time). HPLC=97.65%.

Compound (20): 40 mg, White solid (25% yield) 1H NMR (400 MHZ, DMSO) δ:0.92 (t, 3H), 1.50-1.59 (m, 2H), 2.82 (t, 2H), 3.71 (s, 3H), 4.39 (s, 2H), 5.84 (s, 1H), 6.85 (d, 2H), 7.24 (d, 2H), 12.66 (brs, 1H). LCMS (Method-3): Rₜ=2.81 min; [M+H]-331 (5 min run time). HPLC=98.85%.

Compound (21): 30 mg, White solid (19% yield). 1H NMR (400 MHz, DMSO) δ:0.94 (t, 3H), 1.53-1.59 (m, 2H), 2.86 (t, 2H), 4.42 (s, 2H), 5.89 (s, 1H), 7.16-7.28 (m, 3H), 12.73 (brs, 1H); LCMS (Method-1): Rₜ=1.52 min; [M+H]=337 (3 min run time). HPLC=99.75%.

Compound (22): 30 mg, White solid (19% yield). 1H NMR (400 MHZ, DMSO) δ:0.92 (t, 3H), 1.51-1.57 (m, 2H), 2.83 (t, 2H), 4.39 (s, 2H), 5.83 (s, 1H), 7.00-7.04 (m, 1H), 7.21-7.27 (m, 1H), 7.37-7.43 (m, 1H), 12.73 (brs, 1H); LCMS (Method-1): Rₜ=2.37 min; [M+H]=337 (3 min run time). HPLC-99.31%.

Compound (23): 35 mg, White solid (22% yield). 1H NMR (400 MHZ, DMSO) δ:0.92 (t, 3H), 1.50-1.57 (m, 2H), 2.86 (t, 2H), 4.36 (s, 2H), 5.89 (s, 1H), 7.09 (t, 2H) 7.37-7.43 (m, 1H), 12.72 (brs, 1H); LCMS (Method-1): Rₜ=2.32 min; [M+H]=337 (5 min run time). HPLC-98.74%.

Compound (24): 70 mg, White solid (17% yield). 1H NMR (400 MHZ, DMSO) δ:0.94 (t, 3H), 1.54-1.60 (m, 2H), 2.85 (t, 2H), 4.35 (s, 2H), 5.85 (s, 1H), 7.55 (s, 2H), 12.71 (brs, 1H) LCMS (Method-1): Rₜ=1.829 min; [M+H]=291 (5 min run time). HPLC=98.45%.

Compound (25): 30 mg, White solid (19% yield). 1H NMR (400 MHZ, DMSO) δ:0.91 (t, 3H), 1.52-1.62 (m, 2H), 2.33 (s, 6H), 2.82 (t, 2H), 4.49 (s, 2H), 5.87 (s, 1H), 7.04-7.13 (m, 3H), 12.69 (brs, 1H); LCMS (Method-2): Rₜ=1.69 min; [M+H]=329 (3 min run time). HPLC=99.6%.

Compound (26) 230 mg, White solid (27.38% yield). 1H NMR (400 MHZ, DMSO) δ:0.91 (t, 3H), 1.50-1.55 (m, 2H), 2.82 (t, 2H), 4.41 (s, 2H), 5.88 (s, 1H), 7.20-7.24 (m, 1H), 7.33-7.40 (m, 2H), 12.77 (brs, 1H); LCMS (Method-1): Rₜ=1.58 min; [M+H]=353 (3 min run time). HPLC=99.82%.

Compound (27) 234 mg, White solid (29.9% yield) ¹H NMR (400 MHZ, DMSO) δ:0.88 (t, 3H), 1.49-1.54 (m, 2H), 2.15 (s, 3H), 2.29 (s, 3H), 2.74 (t, 2H), 4.36 (s, 2H), 5.81 (s, 1H), 6.86 (s, 1H), 6.98-6.99 (m, 1H), 7.06-7.08 (m, 2H), 12.77 (brs, 1H); LCMS (Method-1): Rₜ=1.63 min; [M+H]=329 (3 min run time). HPLC=99.79%.

Compound (28) 40 mg, White solid (12% yield). 1H NMR (400 MHZ, DMSO) δ:0.93 (t, 3H), 1.55-1.61 (t, 2H), 2.83 (t, 2H) 3.03 (t, 2H), 3.47 (t, 2H), 5.87 (s, 1H), 7.00-7.42 (m, 3H), 12.69 (s, 1H). LCMS (Method-1): Rₜ=1.67 min; [M+H]=351. HPLC=98.34%.

### b) Compounds of Formula (I) Wherein R2 is an Optionally Substituted Alkyl

Compounds 29-46 were synthesized according to Scheme 1 from the description.

[chemical structure]

In particular, Scheme 4 below was followed wherein R2 is selected from cycloalkyl, benzyl, optionally substituted alkyl such as 3,3,3-trifluoropropyl —CH₂OCH₃ or —CH₂CH₂OCH₂A wherein A is selected from methyl, or optionally substituted CH₂Ph.

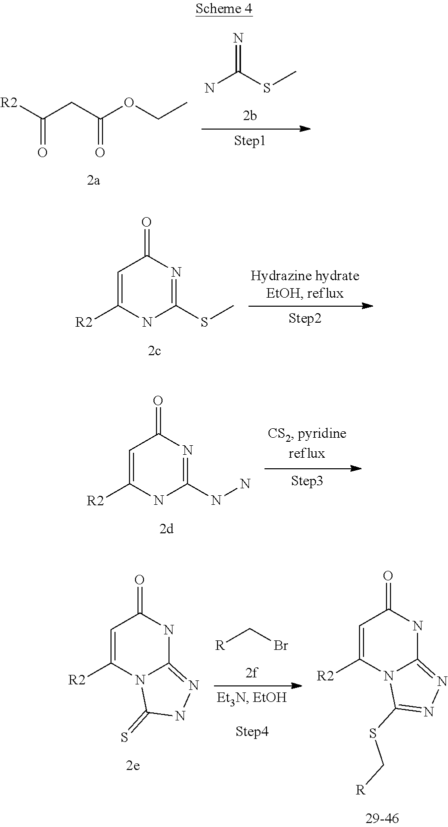

### Preparation of Compound (29)

### Scheme 4, Step 1: Representative Preparation of Intermediate (2c) with R2=Me

To a stirred solution of Ethylacetoacetate (2a, R2=Me) [3 g, 1.0 eq] in water [24 mL, 8 Vol] was added 2-methyl-2-thiourea (2b) [7.0 g, 1.1 eq], followed by sodium carbonate [3.9 g, 1.6 eq]. The reaction was stirred at room temperature for 48 hr. A white solid was precipitated, filtered and washed with diethyl ether (2 volume) and dried under reduced pressure to afford intermediate (2c R2=Me) as a white solid (1.8 gm, 50% yield). 1H NMR (400 MHZ, DMSO-d6): δ 2.11 (s, 3H), 2.41 (s, 3H), 5.84 (s, 1H). LCMS (Method-2): product: RT=1.57 min, m/z=157 (M+H).

### Scheme 4, Step 2: Representative Preparation of Intermediate (2d) with R2=Me

To a stirred solution of intermediate (2c, R2=Me) [800 mg, 1.0 eq] in Ethanol [8 mL, 10 Vol] was added 100% Hydrazine hydrate [4.9 mL, 30 eq], at room temperature. The reaction mass was heated at 80° C. for 24 hr. Reaction mass was monitored by TLC/LCMS. After complete consumption of starting material, the reaction mixture was distilled under reduced pressure, co-distilled with toluene. The solid was recrystallized with ethanol, white solid was precipitated. Filtered the solid, washed the solid with ethanol and dried under reduced pressure to afford intermediate (2d, R2=Me) as a white solid (420 mg, 58% yield). LCMS (Method-2): Product: RT=0.8 min, m/z=141 (M+H).

### Scheme 4, Step 3: Representative Preparation of Intermediate (2e) with R2=Me

To a stirred solution of intermediate (2d, R2=Me) [400 mg, 1.0 eq] in pyridine [11 mL, 27 Vol] was added carbon disulfide [2.2 mL, 5.5 Vol], at room temperature. The reaction mass was heated at 100° C. for 24 hr. Reaction mass was monitored by TLC/LCMS. After completion of starting material the reaction mixture was distilled under reduced pressure and co-distilled with toluene. The solid was recrystallized with ethanol, white solid was precipitated. Filtered the solid, washed the solid with ethanol and dried under reduced pressure to afford intermediate (2e, R2=Me) as a white solid (150 mg, 29% of yield). 1H NMR (400 MHZ, DMSO-d6): δ 2.11 (s, 3H), 5.28 (s, 1H), 5.83 (s, 1H). LCMS (Method-2): product: RT=0.52 min, m/=183.2 (M+H).

### Preparation of Compound (29) by Scheme 4, Step 4

To a stirred solution of intermediate (2e, R2=Me) [350 mg, 1.0 eq] in ethanol [15 mL, 50 Vol] was added Triethyl amine [0.3 mL, 1.1 eq], followed by 2, 5-diflurobenzyl bromide [397 mg, 1.0 eq] at room temperature. The reaction mass was stirred at RT for 24 h. Reaction mass was monitored by TLC/LCMS. After completion of starting material the reaction mixture was distilled under reduced pressure. Then water was added and extracted with 20% MeOH/DCM 3×10 Vol. The organic layer was washed with brine solution and dried over Na₂SO₄ and concentrated under reduced pressure. The crude was purified by column chromatography 100-200 mesh silica gel eluting with Ethyl acetate/Hexane. The compound was again purified by Preparative TLC and affords compound (29) the solid (35 mg, 6% yield) (R═CH₃): 1H NMR (400 MHZ, DMSO-d6): δ 2.66 (s, 3H), 4.36 (s, 2H), 5.94 (s, 1H), 7.18-7.25 (m, 3H). LCMS (Method-2): product: RT=1.44 min, m/z=309.3 (M+H)

Compounds 30-35 were prepared similarly to compound 29 via Scheme 4, steps 1˜4 starting from various substituted intermediates 2a:

Compound (30), starting from intermediate (2a, (R2=CH₂CH₂OCH₂Ph): 13% yield. 1H NMR (400 MHZ, DMSO-d6): δ 3.24-3.27 (t, 2H), 3.71-3.73 (t, 2H), 4.37 (s, 2H), 4.48 (s, 2H), 5.96 (s, 1H), 7.17-7.31 (m, 8H), 12.75 (bs, 1H). LCMS (Method-5): product: RT=2.53 min, m/z=429.1 (M+H). HPLC: 99.18%.

Compound (31) starting from intermediate (2a, R2=CH₂OCH₃): 12% yield. 1H NMR (400 MHz, DMSO-d6): δ 3.31 (s, 3H), 4.27 (s, 2H), 4.62 (s, 2H), 6.06 (s, 1H), 7.12-7.19 (m, 3H). LCMS (Method-5): product: RT=2.14 min, m/z=339.1 (M+H).

Compound (32) starting from intermediate (2a, (R2=CH₂CH₂OCH₂ (4-Cl-Ph)): 5% yield. 1H NMR (400 MHZ, DMSO-d6): δ 3.24-3.27 (t, 2H), 3.70-3.73 (t, 2H), 4.37 (s, 2H), 4.47 (d, 2H), 5.94 (s, 1H), 7.17-7.24 (m, 3H), 7.28-7.30 (d, 2H), 7.36-7.38 (d, 2H), 12.75 (s, 1H). LCMS (Method-5): product: RT=2.66 min, m/z=463.1 (M+H). HPLC: 96.47%.

Compound (33), starting from intermediate (2a, (R2=CH₂CH₂OCH₂ (2-Cl-Ph)): 25% yield. 1H NMR (400 MHZ, DMSO-d6): δ 3.27-3.31 (t, 2H), 3.78-3.81 (t, 2H), 4.38 (s, 2H), 4.55 (s, 2H), 5.98 (s, 1H), 7.14-7.23 (m, 3H), 7.24-7.31 (d, 2H), 7.40-7.42 (t, 2H), 12.75 (bs, 1H). LCMS (Method-5): product: RT=2.63 min, m/z=463.1 (M+H). HPLC: 99.64%.

Compound (34), starting from intermediate (2a, (R2=CH₂CH₂OCH₂ (3-Cl-Ph)): 15% yield. 1H NMR (400 MHz, DMSO-d6): δ 3.26-3.29 (t, 2H), 3.72-3.75 (t, 2H), 4.38 (s, 2H), 4.49 (s, 2H), 5.97 (s, 1H), 7.15-7.37 (m, 7H), 12.76 (bs, 1H). LCMS (Method-5): product: RT=2.65 min, m/z=463 (M+H). HPLC: 99.11%.

Compound (35), starting from intermediate (2a, (R2=CH₂CH₂OCH₃): 13% yield. 1H NMR (400 MHZ, DMSO-d6): δ 3.24-3.27 (t, 2H), 3.71-3.73 (t, 2H), 4.37 (s, 2H), 4.48 (s, 2H), 5.96 (s, 1H), 7.17-7.31 (m, 8H), 12.75 (bs, 1H). LCMS (Method-5): product: RT=2.53 min, m/z=429.1 (M+H). HPLC: 99.18%.

### Preparation of Compound (36)

### Scheme 4, Step 1: Representative Preparation of Intermediate (2c) with R2=Me

To a stirring solution of Ethylacetoacetate (2a, R2=Me) (5 g, 38.462 mmol) and 2-methyl-2-thiopseudourea sulphate (12.83 g, 46.15 mmol) in water (10 mL), Na₂CO₃ (10.19 g, 96.15 mmol) was added and stirred at room temperature for 48 h. A white precipitate was formed, which was filtered and triturated with diethyl ether to afford desired intermediate (2c, R2=Me) as white solid in 86% yield, 5.2 g. 1H NMR (400 MHZ, DMSO-d6) δ 12.50 (br s, 1H), 5.95 (s, 1H), 2.46 (s, 3H), 2.32 (s, 3H)

### Scheme 4, Step 2: Representative Preparation of Intermediate (2c) with R2=Me

Intermediate (2c, R2=Me) (1.3 g, 8.33 mmol) was taken in a sealed tube and to it was added 1(M) Hydrazine in THF (18 ml, 16.66 mmol) and the reaction mixture was heated at 100° C. for 16 hours. TLC showed generation of polar spot. Crude LCMS data confirmed the formation of product. The reaction mixture was evaporated in vacuo and triturated with diethyl ether to afford intermediate (2d, R═Me) as pink solid in 56% yield, 650 mg, 1H NMR (400 MHZ, DMSO-d6) δ 8.32 (br s, 2H), 5.36 (s, 1H), 4.54 (br, 2H), 1.99 (s, 3H)

### Scheme 4, Step 3: Representative Preparation of Intermediate (2c) with R2=Me

To a stirred solution of intermediate (2d, R═Me) (650 mg, 4.64 mmol) in pyridine (11 mL), CS₂ (7 mL, 1 ml/mmol) was added and refluxed for 16 h. The reaction mixture was evaporated in vacuum, azeotroped with toluene and finally triturated with Ethanol and filtered to afford intermediate (2e, R═Me) as a yellow solid in 53% yield, 450 mg, 1H NMR (400 MHZ, DMSO-d6) δ 13.69 (s, 1H), 12.49 (s, 1H), 5.83 (s, 1H), 2.85 (s, 3H)

### Preparation of Compound (36) by Scheme 4, Step 4

To a stirred solution of intermediate (2e, R═Me) (230 mg, 1.26 mmol) and TEA (0.46 mL, 3.16 mmol) in EtOH (12 mL), 2-chloro-6-fluoro benzyl bromide (22 5 mg, 1.011 mmol) was added at 0° C. and stirred at that temperature for an hour. TLC showed generation of non-polar spots (desired and di-substituted compounds). The reaction mixture was evaporated in vacuum, and then diluted with dichloromethane and extracted with water two times and then the organic layer was passed over anhydrous sodium sulphate. The organic layer was dried, concentrated and purified with Combi-flash chromatography (4% MeOH-DCM) to afford the compound 36 as a white solid in 22% yield, 91 mg. N.B. HMBC confirmed the right region-isomer formation. ¹H NMR (400 MHZ, DMSO-d6) δ 12.75 (brs, 1H), 7.33-7.40 (m, 2H), 7.20-7.29 (t, 1H), 5.98 (s, 1H), 4.4 (s, 2H), 2.54 (S, 3H). LCMS (Method-4): Rₜ=2.16 min; [M+H]-325 (5 min run time). HPLC=99.82%.

Compounds 37-46 were prepared similarly to compound 36 via Scheme 4, steps 1˜4 starting from various substituted intermediates 2a:

Compound (37) starting from intermediate (2a, (R2=CH₂CH₃) 1H NMR (400 MHZ, DMSO-d6) δ 12.6 (brs, 1H), 7.34-7.41 (m, 2H), 7.21-7.25 (t, 1H), 5.88 (s, 1H), 4.43 (s, 2H), 2.93-2.98 (q, 2H), 1.12-1.16 (t, 3H). LCMS (Method-1): Rₜ=1.64 min; [M+H]-339 (3 min run time). HPLC=95.18%.

Compound (38) starting from intermediate (2a, (R2=CH₂CH₂CH₂CH₃) 1H NMR (400 MHZ, DMSO-d6) δ 12.76 (brs, 1H), 7.37 (m, 2H), 7.22 (t, 1H), 5.91 (s, 1H), 4.42 (s, 2H), 2.84 (t, 2H), 1.48 (m, 2H), 1.32 (m, 2H), 0.87 (3, 3H). LCMS (Method-1): Rₜ=1.60 min; [M+H]=367.3 (3 min run time). HPLC=95.68%.

Compound (39) starting from intermediate (2a, (R2=cyclopropyl) 1H NMR (400 MHZ, DMSO-d6) δ 12.6 (brs, 1H), 7.35-7.42 (m, 2H), 7.23-7.27 (t, 1H), 5.81 (s, 1H), 4.51 (s, 2H), 2.50 (s, 1H), 1.02-1.04 (d, 2H), 0.96 (brs, 1H). LCMS (Method-4): Rₜ=2.32 min; [M+H]=351 (3 min run time). HPLC=95.76%.

Compound (40) starting from intermediate (2a, (R2-CH₂CH(CH₃) 2) 1H NMR (400 MHZ, DMSO-d6) δ 12.81 (brs, 1H), 7.34-7.41 (m, 2H), 7.20-7.25 (t, 1H), 4.43 (s, 2H), 2.66-2.68 (d, 2H), 1.79-1.84 (m, 1H), 0.86-0.88 (d, 6H). LCMS (Method-5): Rₜ=1.62 min; [M+H]-367 (3 min run time). HPLC=97.66%.

Compound (41) starting from intermediate (2a, (R2-CH₂-cyclopropyl) 1H NMR (400 MHZ, DMSO-d6) δ 12.82 (brs, 1H), 7.34-7.41 (m, 2H), 7.21-7.25 (t, 1H), 6.08 (s, 1H), 4.39 (s, 2H), 2.80-2.82 (d, 2H), 0.95 (brs, 1H), 0.55-0.57 (d, 2H), 0.13-0.14 (d, 2H). LCMS (Method-4): Rₜ=2.42 min; [M+H]=365 (5 min run time). HPLC-97.46%.

Compound (42) starting from intermediate (2a, (R2-CH₂OCH₃) ¹H NMR (400 MHZ, DMSO-d6) δ 12.9 (brs, 1H), 7.34-7.42 (m, 2H), 7.20-7.24 (t, 1H), 6.1 (s, 1H), 4.67 (s, 2H), 4.37 (s, 2H), 3.32 (s, 3H). LCMS (Method-4): Rₜ=2.19 min; [M+H]=355 (5 min run time). HPLC=96.03%.

Compound (43) starting from intermediate (2a, (R2=CH₂CH₂OCH₂Ph) ¹H NMR (400 MHZ, DMSO-d6) δ 12.82 (brs, 1H), 7.34 (m, 8H), 5.98 (s, 1H), 4.47 (s, 2H), 4.39 (s, 2H), 3.71 (t, 2H), 3.29 (t, 2H). LCMS (Method-6): Rₜ=1.82 min; [M+H]=445.3 (3 min run time). HPLC=98.23%.

Compound (44) starting from intermediate (2a, (R2=CH₂CH₂CF₃) ¹H NMR (400 MHZ, DMSO-d6) δ 7.33-7.40 (m, 2H), 7.20-7.24 (t, 1H), 6.04 (s, 1H), 4.39 (s, 2H), 3.16-3.19 (t, 2H), 2.6-2.67 (m, 2H). LCMS (Method-4): Rₜ=2.43 min; [M+H]=407 (5 min run time). HPLC=99.06%.

Compound (45) starting from intermediate (2a, (R2-CH₂Ph) ¹H NMR (400 MHZ, DMSO-d6) δ 12.90 (brs, 1H), 7.34-7.43 (m, 5H), 7.19-7.31 (m, 3H), 5.44 (s, 1H), 4.34 (s, 2H), 4.26 (s, 2H). LCMS (Method-5): Rₜ=1.60 min; [M+H]=401 (3 min run time). HPLC=99.46%.

Compound (46) starting from intermediate (2a, (R2-CH₂Ph) and replacing 2-chloro-6-fluoro benzyl bromide with 4-cyanobenzylbromide in step 4:60 mg, White solid (25% yield). 1H NMR (400 MHZ, DMSO) δ:4.34 (s, 2H), 4.41 (s, 1H), 5.39 (s, 1H), 7.22-7.37 (m, 5H), 7.47 (d, 2H), 7.75 (d, 2H), 12.69 (s, 1H). LCMS (Method-1): Rₜ=1.56 min; [M+H]=374. HPLC=98.93%.

When not available commercially the respective intermediates 2a for compounds 30-46 were sourced as followed:

Intermediates 2a for compounds for compounds 30, 32-34 and 43 were synthesized according to Scheme 5.

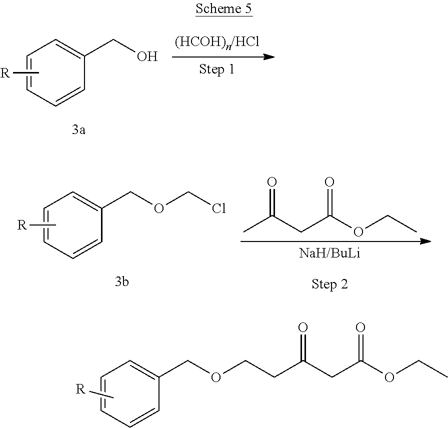

### Scheme 5, Step 1 Preparation of Intermediates (3b)

4-Chloro benzyl alcohol intermediate (3a, R═4-CI) [10 g, 1.0 eq.] in dry Hexane [100 ml, 10 Vol.] was stirred at room temperature for 10 minutes then para formaldehyde [2.7 g, 1.0 eq.] was added and stirred at RT for 10 minutes. The reaction mass was cooled to 0° C. and dry HCl gas was purging for 1. Then the reaction mixture was continued for 24 h at 5° C. The reaction mass was monitored by 1H NMR. The reaction mixture was filtered through sintered funnel dried over Na₂SO₄ and concentrated under reduced pressure to afford intermediate (3b, R═4-Cl) as a liquid (10.9 g, 81% yield). 1H NMR (400 MHZ, DMSO-d6): δ 4.69 (s, 2H), 5.50 (s, 2H), 7.27-7.34 (m, 4H).

3b wherein R is H: purchased from commercial vendor.

For 3b wherein R is 2-chloro, 2-Chloro benzyl alcohol intermediate (3a, R═2-Cl) was used and led to the corresponding intermediate (3b R═2-Cl): 82% yield. 1H NMR (400 MHZ, DMSO-d6): δ 4.84 (s, 2H), 5.57 (s, 2H), 7.24-7.28 (m, 2H), 7.36-7.39 (t, 1H), 7.43-7.45 (t, 1H).

For 3b wherein R is 3-chloro, 3-Chloro benzyl alcohol intermediate (3a, R═3-Cl) was used and led to the corresponding intermediate compound (3b R═3-Cl): 85% yield. 1H NMR (400 MHZ, DMSO-d6): δ 4.70 (s, 2H), 5.51 (s, 2H), 7.21-7.29 (m, 1H), 7.29-7.30 (d, 2H), 7.35 (s, 1H).

### Scheme 5, Step 1 Preparation of Intermediates (2a, R2=CH₂CH₂OCH₂Ar)

NaH [1.6 g, 1.05 eq] was taken under nitrogen atmosphere and dry THF [50 mL, 10 Vol] was added slowly. The reaction mixture was cooled to 0° C. then ethyl acetoacetate [5 g, 1.0 eq] in dry THF added drop wise. After stirring for 10 minutes, nBuLi 2.5 M [17 ml, 1.1 eq] was added drop wise at 0° C. The reaction mixture was stirred for 15 minutes then 3a (R═4-Cl) [6.5 g, 0.9 eq] was added drop wise. The reaction mixture was brought to room temperature and stirred for 1-1.5 hr then quenched with ice cold water at 0° C. followed by 6N HCl, pH=2-3. The reaction mixture was extracted with ethyl acetate (3×10 Volume). The combined organic layer was washed with brine solution and dried over Na₂SO₄ and concentrated under reduced pressure. The crude material was purified by column chromatography 100-200 mesh silica gel eluting with hexane. At 20% EA/Hex pure fraction were collected and distilled under reduced pressure to afford pure intermediate 2a (R2=CH₂CH₂OCH₂-(4-Cl-Ph)) as liquid (3.5 g, 32% yield). 1H NMR (400 MHZ, DMSO-d6): δ 1.21-1.27 (t, 3H), 1.71-1.74 (t, 1H), 2.80-2.83 (t, 2H), 3.46 (s, 1H), 3.71-3.74 (t, 2H), 4.14-4.20 (q, 2H), 4.45 (s, 2H), 4.65-4.66 (d, 2H), 7.22-7.33 (m, 4H), The following intermediates 2a were synthesized using the same approach from 3b as described above.

For intermediate 2a (R2=CH₂CH₂OCH₂-(2-Cl-Ph)) 39% yield. 1H NMR (400 MHZ, DMSO-d6): δ 1.21-1.29 (t, 3H), 1.90-93-1.74 (t, 1H), 2.26 (s, 1H), 3.43 (s, 1H), 3.71-3.74 (t, 2H), 4.15-4.22 (q, 1H), 4.77-4.79 (d, 2H), 7.23-7.29 (m, 2H), 7.34-7.36 (d, 1H), 7.46-7.48 (d, 1H).

For intermediate 2a (R2=CH₂CH₂OCH₂Ph) 30% yield. 1H NMR (400 MHZ, DMSO-d6): δ 1.21-1.24 (t, 3H), 2.02 (s, 2H), 4.11-4.20 (m, 2H), 4.68-4.69 (d, 1H), 4.73 (s, 1H), 5.21 (s, 2H), 7.24-7.36 (m, 5H).

For intermediate 2a (R2=CH₂CH₂OCH₂-(3-Cl-Ph)) 21% yield. 1H NMR (400 MHZ, DMSO-d6): δ 1.21-1.27 (t, 3H), 2.24 (s, 2H), 3.49-3.54 (t, 2H), 4.11-4.20 (q, 2H), 4.58 (s, 1H), 4.77 (s, 1H), 5.28 (s, 2H), 7.19-7.22 (m, 1H), 7.24 (s, 1H), 7.32-7.36 (t, 1H), 7.41-7.48dd, 1H).

Intermediate 2a (R2=CH₂CH₂OMe) for producing compound 35 was prepared as follows: To a stirred solution of Potassium ethyl malonate [12.5 g, 1.0 eq] in DCM [10 Vol] was added TEA [12.0 ml, 1.1 eq], The reaction mixture as cooled to 10° C. under N₂ atmosphere and stirred for 30 min. magnesium chloride [1.2 eq] was added and stirred at RT for 2.5 hrs. The reaction mixture was cooled to 0° C. and 3-methoxy propyl chloride (0.5 eq) slowly added and stirred at RT for 18 hrs. The reaction mixture was distilled under reduced pressure, then 15% HCl was added and extracted with DCM (3×10 Vol). The organic layer was washed with brine solution and dried over Na₂SO₄ and concentrated under reduced pressure. The crude compound was purified by column chromatography 100-200 mesh silica gel eluting with EA/Hex. At 10-20% EA/Hexane pure fraction were collected and distilled under reduced pressure to afford intermediate 2a (R═CH₂CH₂OMe) (2.9 g, 23% yield). 1H NMR (400 MHZ, DMSO-d6): δ 1.16-1.20 (t, 3H), 2.72-2.75 (t, 2H), 3.22 (s, 3H), 3.51-3.54 (t, 2H), 3.59 (s, 2H), 4.06-4.11 (q, 2H). Intermediate 2a (R2=CH₂CH₂CF₃) for producing compound 44 was prepared as follows: To a stirred solution of 4,4,4-trifluorobutyric acid (1.0 g, 6.94 mmol) in 15 ml DCM was added meldrum's acid (0.986 g, 6.94 mmol) and stirred at room temperature for 10 minutes. To it was added DCC and DMAP and the reaction mixture was stirred at room temperature for 16 hours. After the completion of the reaction the reaction mixture was filtered through celite bed and it was extracted with 1 (N) HCl. The combined organic part was passed over anhydrous sodium sulphate and was evaporated under vacuum and the crude was taken into next step without further purification. The crude was then dissolved in methanol and catalytic p-TSA was added and it was refluxed for 1.5 hours. The reaction mixture was evaporated to dryness and the crude material used directly without further purification.

### c) Compounds of Formula (I) Wherein R5 is S, and n=0; where R2 and R3 are Both Alkyl; where R5 is NH

Compounds 47-49 were synthesized according to Scheme 1 from the description.

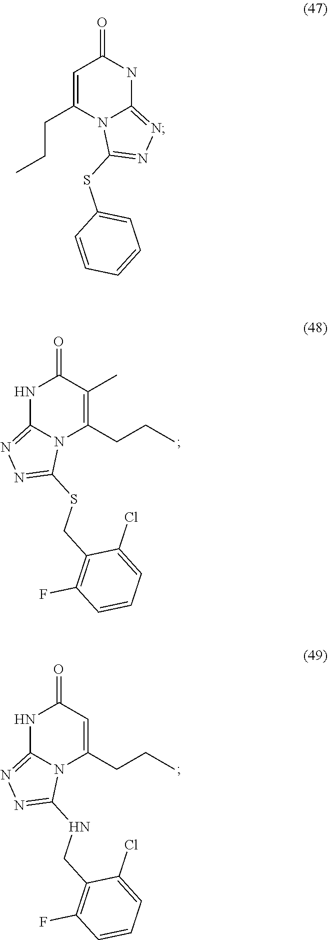 [SMILES: CCCc1cc(=O)[nH]c2nnc(Sc3ccccc3)n12 ; CCCc1c(C)c(=O)[nH]c2nnc(SCc3c(F)cccc3Cl)n12 ; CCCc1cc(=O)[nH]c2nnc(NCc3c(F)cccc3Cl)n12]

Compound 47 was prepared according to the following scheme 6:

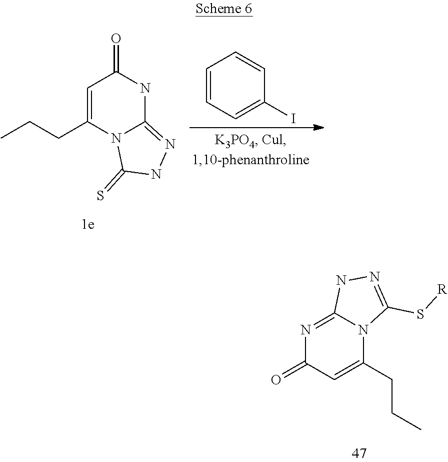

### Scheme 6 to Prepare Compound (47)

To a stirred solution of 5-propyl-3-thioxo-2,8-dihydro-[1,2,4]triazolo[4,3-a]pyrimidin-7 (3H)-one (intermediate 1e from scheme 3, 500 mg, 2.38 mmol) and Iodobenzene (728 mg, 3.57 mmol) in DMSO (5 mL), K3PO₄ (1.51 g, 7.14 mmol), was added and in a sealed tube, then the reaction mixture was degassed for 10 min under Argon atmosphere. Then CuI (45 mg, 0.23 mmol) and 1, 10-phenanthroline (41 mg, 0.23 mmol) were added and reaction mixture was heated for 72 h at 120° C. The reaction mixture was evaporated in gene-vacuo and purified by Combi-flash (0.5% MeOH-DCM) to afford compound 47 as white solid (200 mg, 29% yield). 1H NMR (400 MHZ, DMSO) δ:0.93 (t, 3H), 1.54-1.61 (t, 2H), 2.96 (t, 2H), 5.98 (s, 1H), 7.23-7.38 (m, 5H), 12.90 (s, 1H). LCMS (Method-1): R═2.16 min; [M+H]=287. HPLC=99.75%. Compound 48 was prepared according to the following scheme 7:

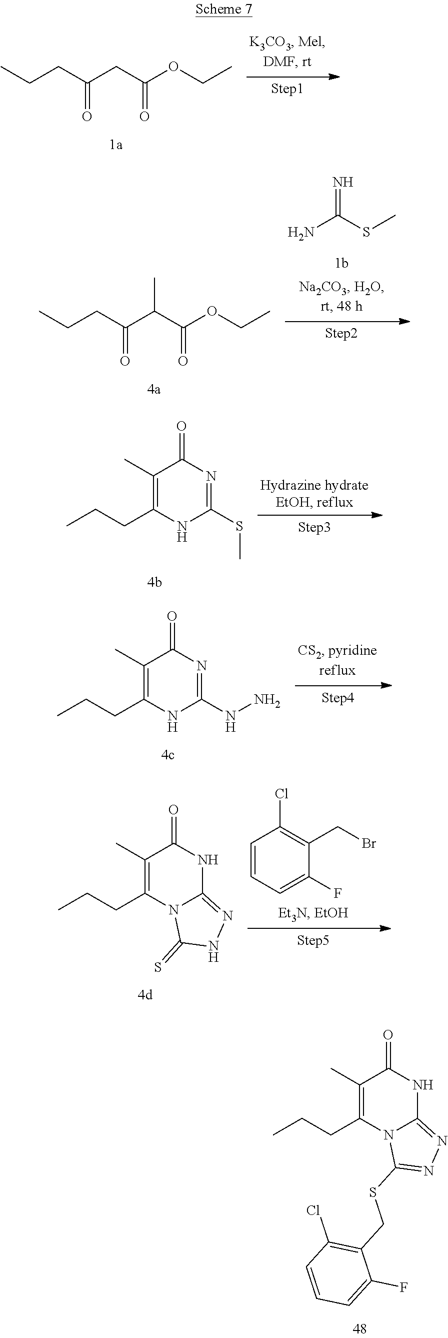

### Scheme 7, Step 1: Intermediate (4a)

To a stirred solution of Ethyl butyrate (5 g, 31.62 mmol) in DMF (100 mL) at 0° C., K₂CO₃ (10.91 g, 79.06 mmol) and Mel (1.97 mL, 31.62 mmol) were added and stir at rt for 48 h. The reaction mixture was partitioned between ethyl acetate and aq. NH4Cl solution. The organic part was dried, concentrated and purified by Combi-flash (eluted with 2% EA-Hex) to afford desired intermediate 4a as colorless liquid in 22.03% yield, 1.2 g. 1H NMR (400 MHZ, DMSO) δ:0.82 (t, 3H), 1.17 (d, 3H), 1.43-1.52 (m, 2H), 2.45 (s, 3H), 3.66-3.71 (m, 1H), 4.07-4.12 (m, 2H);

### Scheme 7, Step 2: Intermediate (4b)

To a stirred solution of intermediate 4a (1.2 g, 6.96 mmol) and 2-Methyl-2-Thiopseudourea hemisulphate (1.06 g, 7.66 mmol) in water (20 mL), Na₂CO₃ (1.18 g, 11.15 mmol) was added and stirred at rt for 48 h. A white precipitate was phase out, and attempted filtration was not successful as white material sticks on the wall of sinterd funnel. So the reaction mixture was partitioned between 10% IPA-MeOH and water. The organic part was dried, concentrated and triturated with ether to afford desired intermediate 4b as white solid in 57.9% yield, 800 mg. 1H NMR (400 MHZ, DMSO) δ:0.90 (t, 3H), 1.54-1.63 (m, 2H), 1.87 (s, 3H), 2.26 (s, 3H), 2.42 (t, 2H), 8.71 (brs, 1H);

### Scheme 7, Step 3: Intermediate (4c)

To a stirring solution of intermediate 4b (800 mg, 4.03 mmol) in Ethanol (10 mL), was added NH₂NH₂·H₂O (2.01 mL, 40.35 mmol) and refluxed for 24 h. The reaction mixture was evaporated to dryness. Neat NH₂NH₂·H₂O (5 mL) was added and refluxed for 16 h. LCMS showed major product formation. The reaction mixture was evaporated to complete dryness and triturated with EtOH and ether to afford the desired intermediate 4c as an off white solid (crude). ¹H NMR (400 MHZ, DMSO) δ:0.85 (t, 3H), 1.49-1.57 (m, 2H), 1.79 (s, 3H), 2.34 (t, 2H), 3.91 (brs, 2H), 7.09 (brs, 1H); LCMS (Method-2): Rₜ=1.32 min; [M+H]=183 (3 min run time, NH₄OAc+MeCN).

### Scheme 7, Step 4: Intermediate (4d)

To a stirring solution of crude 2-hydrazinyl-5-methyl-6-propylpyrimidin-4 (1H)-one (400 mg, 2.19 mmol) in pyridine (10 mL), CS2 (2.2 mL) was added and refluxed for 6 h. The reaction mixture was evaporated in vacuo, azeotroped with toluene and finally triturated with EtOH to afford the crude target compound as off white solid. This crude material (intermediate 4d, 180 mg) was used for the next step with out further purification.

### Scheme 7, Step 5 to Prepare Compound (48)

To a stirred solution of crude intermediate 4d (180 mg, 0.80 mmol) and TEA (167 μL, 1.20 mmol) in EtOH (10 mL), 2-chloro, 6-fluoro benzyl bromide (99 μL, 0.72 mmol) was added and stirred at rt for 18 h. The reaction mixture was evaporated to dryness and partitioned between 10% IPA-DCM and water. The organic part was dried over anhydrous Na₂SO₄, initially purified by Combi-flash (1% MeOH-DCM) and finally by Prep TLC (mobile phase 5% MeOH-DCM) to afford the desired compound (48) as off white solid (20 mg) in 6.79% yield. ¹H NMR (400 MHZ, DMSO) δ:0.85 (t, 3H), 1.46-1.48 (m, 2H), 1.93 (s, 3H), 2.94 (t, 2H), 4.38 (s, 2H), 7.22-7.38 (m, 3H), 12.87 (brs, 1H); LCMS (Method-1): Rₜ=1.58 min; [M+H]=367 (3 min run time, HCOOH+MeCN). HPLC-98.60%.

Compound 49 was prepared according to the following scheme 8:

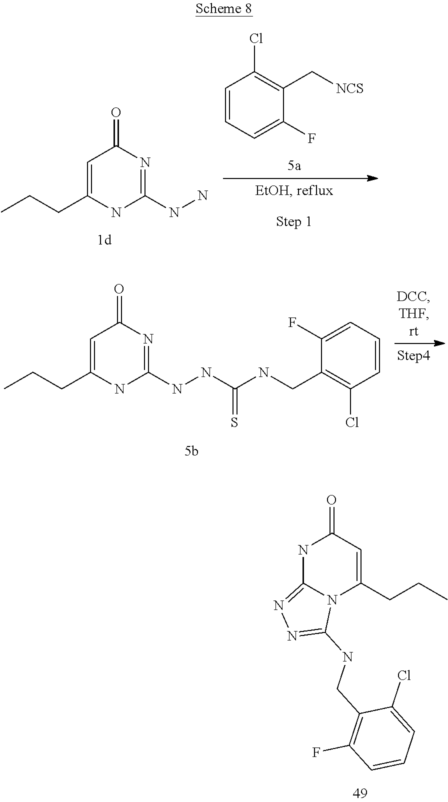

### Scheme 8, Step 1: Intermediate (5b)

To a stirred solution of intermediate 1d (Scheme 3, 700 mg, 4.16 mmol) in EtOH (25 mL), 1-chloro-3-fluoro-2-(isothiocyanatomethyl)benzene (5a, 1.25 g, 6.25 mmol) was added and refluxed for 18 h. The reaction mixture was evaporated to dryness and washed with EtOH. The white solid material was filtered, and concentrated to afford the crude intermediate 5b as off white solid (600 mg). LCMS (Method-3): R1=2.82 min; [M+H]=370 (5 min run time, NH₄OAc+MeCN).

### Scheme 8, Step 2 to Prepare Compound (49)

To a stirred solution of intermediate 5b (600 mg, 1.62 mmol) in Dioxane (25 mL), DCC (402 mg, 1.95 mmol) was added and refluxed for 18 h. TLC showed complete consumption of starting material and generation of two polar spots. The reaction mixture was evaporated to dryness and partitioned between 10% IPA-DCM and saturated NaHCO₃ solution. The organic part was dried over anhydrous Na₂SO₄, initially purified by Combi-flash (1% MeOH-DCM) to afford (49) as off white solid (60 mg) in 11% yield. ¹H NMR (400 MHZ, DMSO) δ:0.79 (t, 3H), 1.53-1.58 (m, 2H), 2.79 (t, 2H), 4.49 (s, 2H), 5.70 (s, 1H), 6.12 (brs, 1H), 7.23-7.40 (m, 3H), 12.22 (s, 1H); LCMS (Method-1): R+=1.46 min; [M+H]=336 (3 min run time, HCOOH+MeCN). HPLC=99.18%.

### d) Compounds of Formula (I) wherein R5 is SO₂ or a Bond

Compounds 50-51 were synthesized according to Scheme 2 from the description.

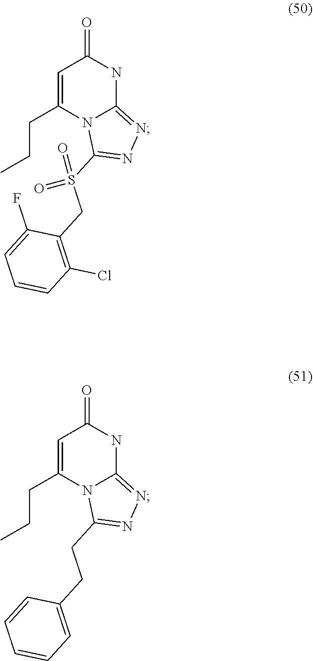 [SMILES: CCCc1cc(=O)[nH]c2nnc(S(=O)(=O)Cc3c(F)cccc3Cl)n12 ; CCCc1cc(=O)[nH]c2nnc(CCc3ccccc3)n12]

Compound 50 was prepared according to the following Scheme 9.

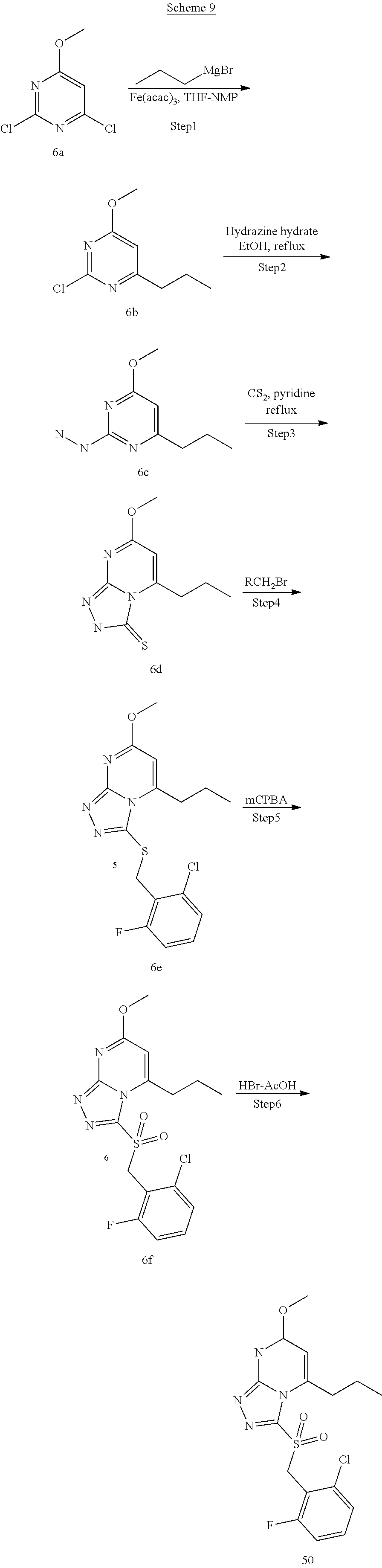

### Scheme 9, Step 1: Intermediate (6b)

To a stirred solution of 2,4-dichloro-6-methoxypyrimidine (6a, 500 mg, 2.81 mmol) and Fe (acac)3 (99.22 mg, 0.28 mmol) in THF (25 mL) at 0° C., PrMgBr (0.41 ml, 2.81 mmol) was added and stir at rt for 48 h. The reaction mixture was partitioned between Ethyl acetate and aq. NH4Cl solution. The organic part was dried, concentrated and purified by Combi-flash (eluted with 2% EA-Hex) to afford desired Intermediate 6b as colorless sticky liquid in 55% yield, 300 mg, 1H NMR (400 MHZ, DMSO) δ:0.88 (t, 3H), 1.61-1.66 (m, 2H), 2.60 (t, 2H), 3.91 (s, 3H), 6.84 (s, 1H); Positive nOe was observed between aromatic proton (at 6.84 ppm) and adjacent-CH₂ (2.60 ppm) proton. LCMS (Method-3): Rₜ=3.41 min; [M+H]=187 (5 min run time, NH40Ac+MeCN).

### Scheme 9, Step 2: Intermediate (6c)

To a stirring solution of Intermediate 6b (300 mg, 1.61 mmol) in dioxane (20 mL), NH2NH2 (4.03 mL, 40.30 mmol, 1M in THF) was added and refluxed for 24 h. The reaction mixture was evaporated to dryness and triturated with ether to afford the desired Intermediate 6c as white solid (150 mg, crude). 1H NMR (400 MHZ, DMSO) δ:0.88 (t, 3H), 1.57-1.66 (m, 2H), 2.40 (t, 2H), 3.81 (s, 3H), 4.10 (brs, 2H), 5.90 (s, 1H), 7.92 (s, 1H).

### Scheme 9, Step 3: Intermediate (6d)

To a stirring solution of crude intermediate 6c (1 g, 5.49 mmol) in pyridine (25 mL), CS2 (5.5 mL) was added and refluxed for 6 h. The reaction mixture was evaporated in vacuo, azeotroped with toluene and finally triturated with EtOH to afford the crude target Intermediate 6d as pale yellow solid (600 mg). This crude material (600 mg) was used for the next step with out further purification. 1H NMR (400 MHZ, DMSO) δ:0.95 (t, 3H), 1.70-1.76 (m, 2H), 3.55 (t, 2H), 3.92 (s, 3H), 6.35 (s, 1H), 14.09 (s, 1H).

### Scheme 9, Step 4: Intermediate (6e)

To a stirred solution of crude Intermediate 6d (100 mg, 0.44 mmol) and TEA (93 μL, 0.67 mmol) in EtOH (10 mL), 2-chloro, 6-fluoro benzyl bromide (71 μL, 0.44 mmol) was added and stirred at rt for 18 h. The reaction mixture was evaporated to dryness and partitioned between 10% IPA-DCM and water. The organic part was dried over anhydrous Na2SO4, initially purified by Combi-flash (1% MeOH-DCM) and finally by Prep TLC (mobile phase 5% MeOH-DCM) to afford intermediate 6e as off white solid (80 mg) in 42.76% yield. 1H NMR (400 MHZ, DMSO) δ:0.91 (t, 3H), 1.56-1.58 (m, 2H), 3.02 (t, 3H), 3.97 (s, 3H), 4.41 (s, 2H), 6.51 (s, 1H), 7.15-7.38 (m, 3H); HMBC confirmed the formation of desired isomer. LCMS (Method-2): Rₜ=1.76 min; [M+H]=367 (3 min run time, NH₄OAc+MeCN). HPLC=99.83%

### Scheme 9, Step 5: Intermediate (6f)

To a stirred solution of 3-((2-chloro-6-fluorobenzyl)thio)-7-methoxy-5-propyl-[1,2,4]triazolo[4,3-a]pyrimidine intermediate 6e (200 mg, 0.54 mmol) in DCM (25 mL), m-CPBA (312 mg, 1.09 mmol, 60%), was added and stirred at rt for 18 hr. The reaction mixture was partitioned between saturated NaHCO₃ solution and DCM. The organic layer was dried, concentrated to afford 80 mg of crude intermediate 6f. LCMS (Method-1): Rₜ=2.18 min; [M+H]=399.

### Scheme 9, Step 6 to Prepare Compound (50)

HBr—AcOH (10 mL) was added to intermediate 6f (80 mg, 0.20 mmol) and heated at 80° C. for 18 h. TLC showed complete consumption of starting material and generation of polar spots. The reaction mixture was evaporated to dryness and partitioned between 10% IPA-DCM and saturated NaHCO₃ solution. The organic part was dried over anhydrous Na₂SO₄, initially purified by Combi-flash (1% MeOH-DCM) to afford compound (50) as a white solid (10 mg) in 13% yield. 1H NMR (400 MHZ, DMSO) δ:0.92 (t, 3H), 1.62-1.66 (m, 2H), 2.90 (t, 2H), 5.41 (s, 2H), 6.06 (s, 1H), 7.37-7.57 (m, 3H). LCMS (Method-1): Rₜ=3.72 min; [M+H]=385. HPLC=94.78%.

Compound 51 was prepared according to the following Scheme 10.

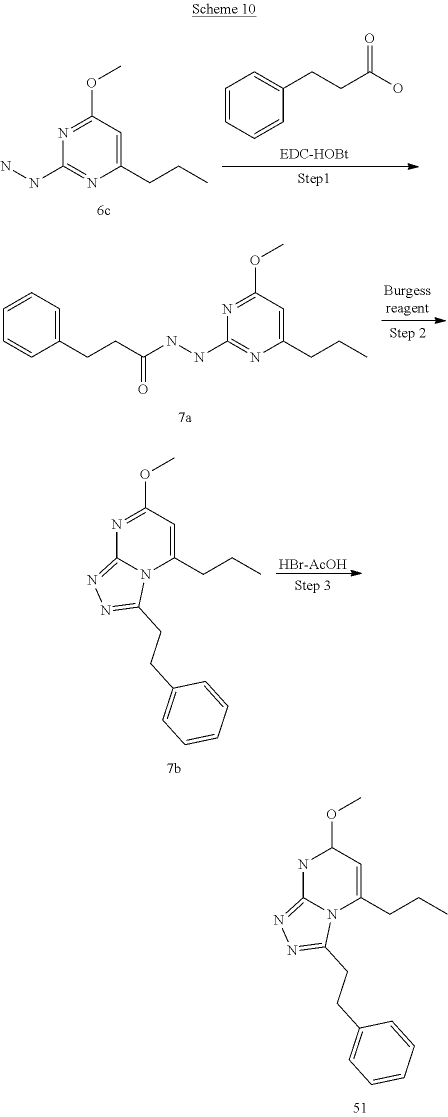 [SMILES: CCCc1cc(OC)nc(NN)n1 ; O=C(O)CCc1ccccc1 ; CCCc1cc(OC)nc(NNC(=O)CCc2ccccc2)n1 ; CCCc1cc(OC)nc2nnc(CCc3ccccc3)n12 ; CCCC1=CC(OC)Nc2nnc(CCc3ccccc3)n21 ; "HBr-AcOH"]

### Scheme 10, Step 1: Intermediate (7a)

To a stirred solution of intermediate 6c (from scheme 9, 700 mg, 3.84 mmol) and 3-phenylpropanoic acid (577 mg, 3.84 mmol) in DMF (25 mL), were added EDC.HCl (1.1 g, 5.76 mmol), HOBt (796 mg, 5.76 mmol) and DIPEA (1.48 g, 11.53 mmol) and stirred for 18 h. The reaction mixture was evaporated to dryness and partitioned between DCM and saturated NaHCO3 solution. The organic part was dried over anhydrous Na₂SO₄, initially purified by Combi-flash (70% EA-Hex) to afford the desired intermediate 7a as off white solid (500 mg) in 42% yield. LCMS (Method-2): Rₜ=1.77 min; [M+H]=315 (3 min run time, NH4OAc+MeCN).

### Scheme 10, Step 1: Intermediate (7b)

To a stirred solution of intermediate 7a (500 mg, 1.59 mmol) in THF (25 ml), Burgess reagent (758 mg, 3.18 mmol) was added and stirred for 48 h. LCMS showed formation of desired product and some unreacted SM. The reaction mixture was evaporated to dryness and partitioned between DCM and saturated NaHCO₃ solution. The organic part was dried over anhydrous Na₂SO₄, initially purified by Combi-flash (60% EA-Hex) to afford the desired intermediate 7b as off white solid (200 mg) in 42% yield. LCMS (Method-2): Rₜ=1.75 min; [M+H]=297 (3 min run time, NH₄OAc+MeCN).

### Scheme 10, Step 3 to Prepare Compound (51)

HBr—AcOH (10 mL) was added to intermediate 7b (200 mg, 0.67 mmol) and heated at 80° C. for 18 h. TLC showed complete consumption of starting material and generation of polar spots. The reaction mixture was evaporated to dryness and partitioned between 10% IPA-DCM and saturated NaHCO₃ solution. The organic part was dried over anhydrous Na₂SO₄, initially purified by Combi-flash (1% MeOH-DCM) to afford the desired compound (51) as off white solid (50 mg) in 26% yield. 1H NMR (400 MHZ, DMSO) δ:0.93 (t, 3H), 1.58-1.64 (m, 2H), 2.86 (t, 2H), 3.12 (t, 2H), 5.83 (s, 1H), 7.20-7.31 (m, 5H), 12.64 (s, 1H); LCMS (Method-2): Rₜ=1.58 min; [M+H]=283 (3 min run time, NH4OAc+MeCN). HPLC=94.22%.

Compounds 52-100 (Table 1) can be easily obtained by a skilled in the art from Schemes 1 and 2, in particular via alkylation of intermediate 1e with an appropriate electophilic reagent such as a benzyl bromide, as per step 4 of Scheme 3. The compounds were acquired for test via commercial sources.

TABLE 1

| Structure | IUPAC Name |
|---|---|
| 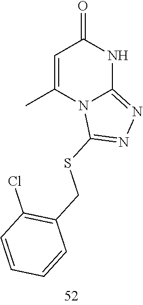 [SMILES: Cc1cc(=O)[nH]c2nnc(SCc3ccccc3Cl)n12] | 3-[(2-chlorobenzyl) sulfanyl]-5-methyl[1,2,4] triazolo[4,3-a] pyrimidin- 7(8H)-one |
| 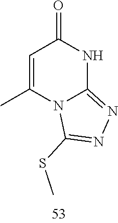 | 5-methyl-3-(methylsulfanyl) [1,2,4]triazolo[4,3-a] pyrimidin-7(8H)-one |
| 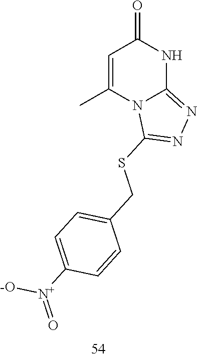 | 5-methyl-3-[(4- nitrobenzyl) sulfanyl][1,2,4]triazolo [4,3-a] pyrimidin- 7(8H)-one |
| 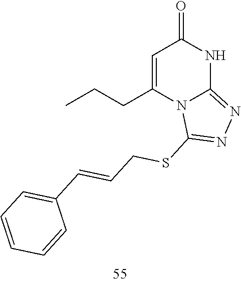 [SMILES: CCCc1cc(=O)[nH]c2nnc(SC/C=C/c3ccccc3)n12] | 3-{[(2E)-3-phenylprop- 2-en-1-yl]sulfanyl}-5- propyl[1,2,4]triazolo[4,3- a]pyrimidin-7(8H)-one |
| 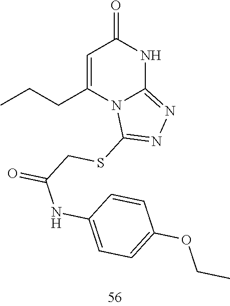 [SMILES: CCCc1cc(=O)[nH]c2nnc(SCC(=O)Nc3ccc(OCC)cc3)n12] | N-(4-ethoxyphenyl)-2- [(7-oxo-5-propyl-7,8- dihydro[1,2,4]triazolo [4,3-a]pyrimidin-3-yl) sulfanyl]acetamide |
| 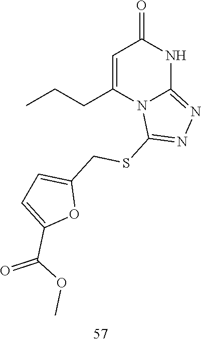 [SMILES: CCCc1cc(=O)[nH]c2nnc(SCc3ccc(C(=O)OC)o3)n12] | methyl 5-{[(7-oxo-5- propyl-7,8-dihydro[1, 2,4]triazolo[4,3- a]pyrimidin- 3-yl)sulfanyl]methyl} furan-2-carboxylate |
| 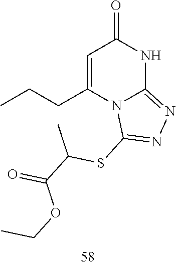 [SMILES: CCCc1cc(=O)[nH]c2nnc(SC(C)C(=O)OCC)n12] | ethyl 2-[(7-oxo-5- propyl-7,8-dihydro [1,2,4]triazolo[4,3- a]pyrimidin-3- yl)sulfanyl] propanoate |
| 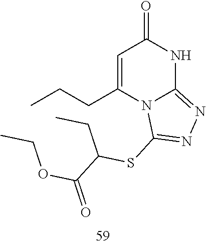 [SMILES: CCCc1cc(=O)[nH]c2nnc(SC(CC)C(=O)OCC)n12] | ethyl 2-[(7-oxo-5- propyl-7,8-dihydro [1,2,4]triazolo[4,3- a]pyrimidin-3- yl)sulfanyl]butanoate |
| 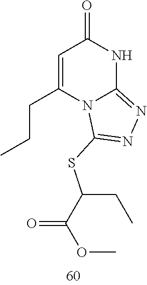 [SMILES: CCCc1cc(=O)[nH]c2nnc(SC(CC)C(=O)OC)n12] | methyl 2-[(7-oxo-5- propyl-7,8-dihydro [1,2,4]triazolo[4,3- a]pyrimidin- 3-yl)sulfanyl]butanoate |
| 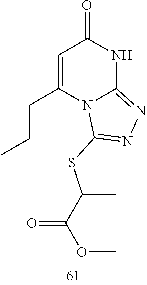 [SMILES: CCCc1cc(=O)[nH]c2nnc(SC(C)C(=O)OC)n12] | methyl 2-[(7-oxo-5- propyl-7,8-dihydro [1,2,4]triazolo[4,3- a]pyrimidin- 3-yl)sulfanyl] propanoate |
| 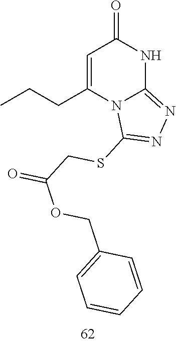 [SMILES: CCCc1cc(=O)[nH]c2nnc(SCC(=O)OCc3ccccc3)n12] | benzyl[(7-oxo-5-propyl- 7,8-dihydro[1,2,4] triazolo[4,3-a]pyrimidin-3- yl)sulfanyl]acetate |
| 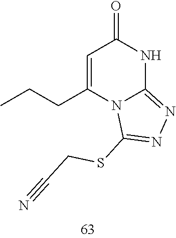 [SMILES: CCCc1cc(=O)[nH]c2nnc(SCC#N)n12] | [(7-oxo-5-propyl-7,8- dihydro[1,2,4]triazolo [4,3-a]pyrimidin-3-yl) sulfanyl]acetonitrile |
| 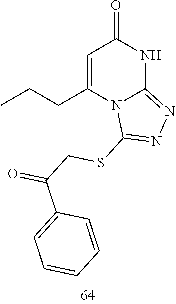 [SMILES: CCCc1cc(=O)[nH]c2nnc(SCC(=O)c3ccccc3)n12] | 3-[(2-oxo-2-phenylethyl) sulfanyl]-5-propyl [1,2,4]triazolo[4,3-a] pyrimidin-7(8H)-one |
| 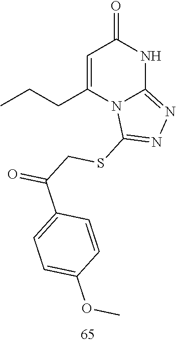 [SMILES: CCCc1cc(=O)[nH]c2nnc(SCC(=O)c3ccc(OC)cc3)n12] | 3-{[2-(4-methoxyphenyl)- 2-oxoethyl]sulfanyl}- 5-propyl[1,2,4]triazolo [4,3-a]pyrimidin-7(8H)- one |
| 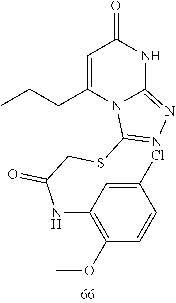 [SMILES: CCCc1cc(=O)[nH]c2nnc(SCC(=O)Nc3cc(Cl)ccc3OC)n12] | N-(5-chloro-2- methoxyphenyl)-2- [(7-oxo-5-propyl- 7,8-dihydro [1,2,4]triazolo[4,3-a] pyrimidin-3-yl) sulfanyl]acetamide |
| 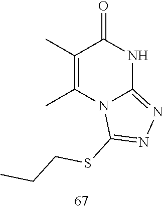 [SMILES: CCCSc1nnc2[nH]c(=O)c(C)c(C)n12] | 5,6-dimethyl-3- (propylsulfanyl)[1,2,4] triazolo[4,3-a] pyrimidin-7(8H)- one |
| 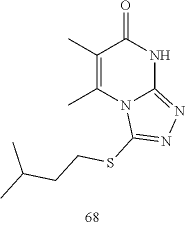 [SMILES: Cc1c(C)n2c(SCCC(C)C)nnc2[nH]c1=O] | 5,6-dimethyl-3-[(3- methylbutyl)sulfanyl] [1,2,4]triazolo[4,3- a]pyrimidin-7(8H)- one |
| 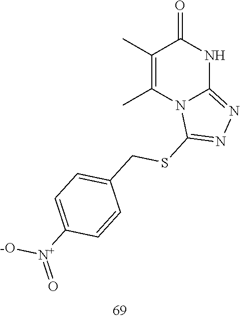 | 5,6-dimethyl-3-[(4- nitrobenzyl)sulfanyl] [1,2,4]triazolo[4,3-a] pyrimidin- 7(8H)-one |
| 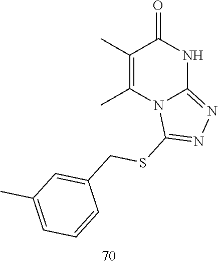 [SMILES: Cc1cccc(CSc2nnc3[nH]c(=O)c(C)c(C)n23)c1] | 5,6-dimethyl-3-[(3- methylbenzyl)sulfanyl] [1,2,4]triazolo[4,3-a] pyrimidin-7(8H)-one |
| 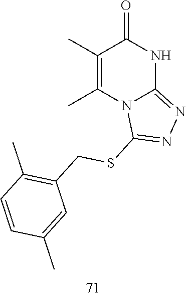 [SMILES: Cc1ccc(C)c(CSc2nnc3[nH]c(=O)c(C)c(C)n23)c1] | 3-[(2,5-dimethylbenzyl) sulfanyl]-5,6-dimethyl [1,2,4]triazolo[4,3-a] pyrimidin-7(8H)-one |
| 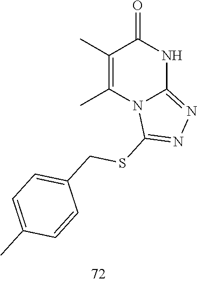 [SMILES: Cc1ccc(CSc2nnc3[nH]c(=O)c(C)c(C)n23)cc1] | 5,6-dimethyl-3-[(4- methylbenzyl)sulfanyl] [1,2,4]triazolo[4,3-a] pyrimidin-7(8H)-one |
| 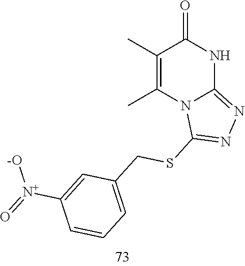 | 5,6-dimethyl-3-[(3- nitrobenzyl)sulfanyl] [1,2,4]triazolo[4,3- a]pyrimidin- 7(8H)-one |
| 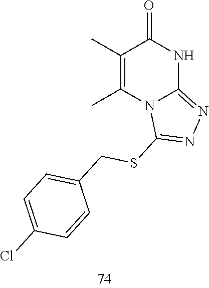 [SMILES: Cc1c(C)n2c(SCc3ccc(Cl)cc3)nnc2[nH]c1=O] | 3-[(4-chlorobenzyl) sulfanyl]-5,6-dimethyl [1,2,4]triazolo[4,3- a]pyrimidin-7(8H)-one |
| 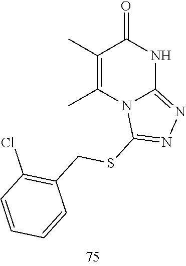 [SMILES: Cc1c(C)n2c(SCc3ccccc3Cl)nnc2[nH]c1=O] | 3-[(2-chlorobenzyl) sulfanyl]-5,6- dimethyl[1,2,4] triazolo[4,3- a]pyrimidin-7 (8H)-one |
| 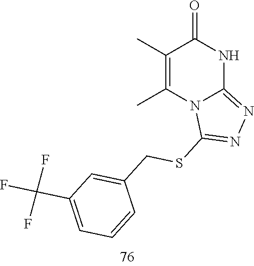 [SMILES: Cc1c(C)n2c(SCc3cccc(C(F)(F)F)c3)nnc2[nH]c1=O] | 5,6-dimethyl-3-{[3- (trifluoromethyl) benzyl]sulfanyl} [1,2,4]triazolo[4,3- a]pyrimidin-7(8H)- one |
| 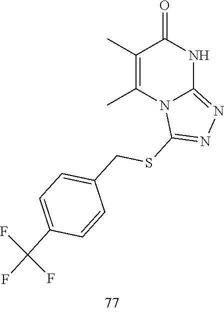 [SMILES: Cc1c(C)n2c(SCc3ccc(C(F)(F)F)cc3)nnc2[nH]c1=O] | 5,6-dimethyl-3-{[4- (trifluoromethyl)benzyl] sulfanyl} [1,2,4]triazolo [4,3-a]pyrimidin-7(8H)- one |
| 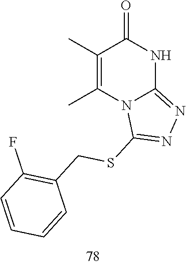 [SMILES: Cc1c(C)n2c(SCc3ccccc3F)nnc2[nH]c1=O] | 3-[(2-fluorobenzyl) sulfanyl]-5,6-dimethyl [1,2,4]triazolo[4,3- a]pyrimidin-7(8H)-one |
| 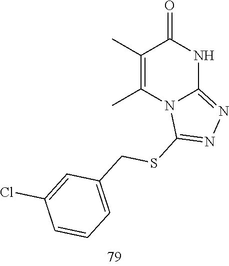 [SMILES: Cc1c(C)n2c(SCc3cccc(Cl)c3)nnc2[nH]c1=O] | 3-[(3-chlorobenzyl) sulfanyl]-5,6-dimethyl [1,2,4]triazolo[4,3- a]pyrimidin-7(8H)- one |
| 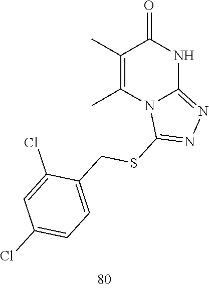 [SMILES: Cc1c(C)n2c(SCc3ccc(Cl)cc3Cl)nnc2[nH]c1=O] | 3-[(2,4-dichlorobenzyl) sulfanyl]-5,6-dimethyl [1,2,4]triazolo[4,3-a] pyrimidin-7(8H)-one |
| 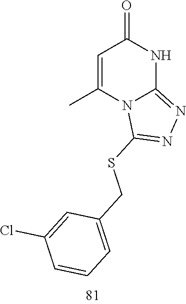 [SMILES: Cc1cc(=O)[nH]c2nnc(SCc3cccc(Cl)c3)n12] | 3-[(3-chlorobenzyl) sulfanyl]-5-methyl[1, 2,4]triazolo[4,3-a] pyrimidin- 7(8H)-one |
| 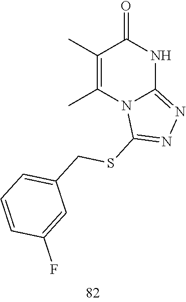 [SMILES: Cc1c(C)n2c(SCc3cccc(F)c3)nnc2[nH]c1=O] | 3-[(3-fluorobenzyl) sulfanyl]-5,6-dimethyl [1,2,4]triazolo[4,3- a]pyrimidin-7(8H)-one |
| 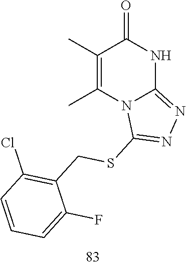 [SMILES: Cc1c(C)n2c(SCc3c(F)cccc3Cl)nnc2[nH]c1=O] | 3-[(2-chloro-6- fluorobenzyl)sulfanyl]- 5,6-dimethyl[1,2, 4]triazolo[4,3- a]pyrimidin-7(8H)-one |
| 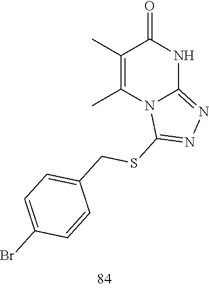 [SMILES: Cc1c(C)n2c(SCc3ccc(Br)cc3)nnc2[nH]c1=O] | 3-[(4-bromobenzyl) sulfanyl]-5,6-dimethyl [1,2,4]triazolo[4,3-a] pyrimidin-7(8H)-one |
| 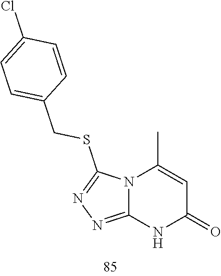 [SMILES: Cc1cc(=O)[nH]c2nnc(SCc3ccc(Cl)cc3)n12] | 3-[(4-chlorobenzyl) sulfanyl]-5-methyl[1,2, 4]triazolo[4,3- a]pyrimidin- 7(8H)-one |
| 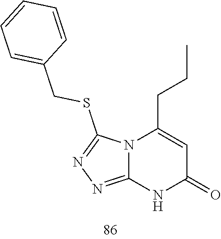 [SMILES: CCCc1cc(=O)[nH]c2nnc(SCc3ccccc3)n12] | 3-(benzylsulfanyl)-5- propyl[1,2,4]triazolo [4,3-a]pyrimidin-7(8H)- one |
| 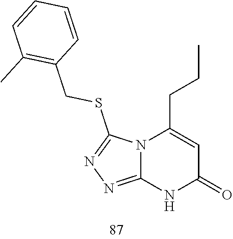 [SMILES: CCCc1cc(=O)[nH]c2nnc(SCc3ccccc3C)n12] | 3-[(2-methylbenzyl) sulfanyl]-5-propyl[1,2, 4]triazolo[4,3- a]pyrimidin- 7(8H)-one |
| 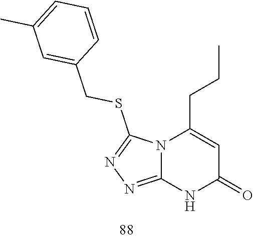 [SMILES: CCCc1cc(=O)[nH]c2nnc(SCc3cccc(C)c3)n12] | 3-[(3-methylbenzyl) sulfanyl]-5-propyl[1,2,4] triazolo[4,3-a]pyrimidin- 7(8H)-one |
| 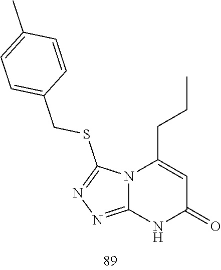 [SMILES: CCCc1cc(=O)[nH]c2nnc(SCc3ccc(C)cc3)n12] | 3-[(4-methylbenzyl) sulfanyl]-5-propyl[1,2,4] triazolo[4,3-a]pyrimidin- 7(8H)-one |
| 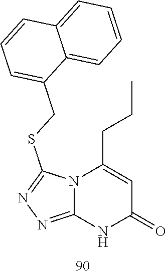 [SMILES: CCCc1cc(=O)[nH]c2nnc(SCc3cccc4ccccc34)n12] | 3-[(naphthalen-1-ylmethyl) sulfanyl]-5-propyl[1,2, 4]triazolo[4,3-a] pyrimidin-7(8H)-one |
| 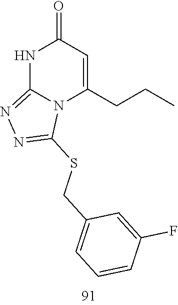 [SMILES: CCCc1cc(=O)[nH]c2nnc(SCc3cccc(F)c3)n12] | 3-[(3-fluorobenzyl) sulfanyl]-5-propyl[1,2, 4]triazolo[4,3-a]pyrimidin- 7(8H)-one |
| 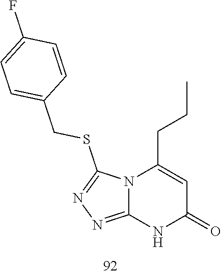 [SMILES: CCCc1cc(=O)[nH]c2nnc(SCc3ccc(F)cc3)n12] | 3-[(4-fluorobenzyl) sulfanyl]-5-propyl[1,2,4] triazolo[4,3-a]pyrimidin- 7(8H)-one |
| 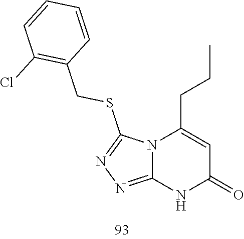 [SMILES: CCCc1cc(=O)[nH]c2nnc(SCc3ccccc3Cl)n12] | 3-[(2-chlorobenzyl) sulfanyl]-5-propyl [1,2,4]triazolo[4,3- a]pyrimidin- 7(8H)-one |
| 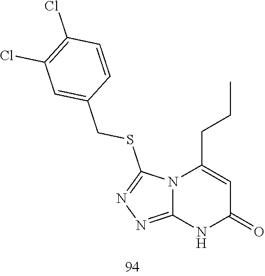 [SMILES: CCCc1cc(=O)[nH]c2nnc(SCc3ccc(Cl)c(Cl)c3)n12] | 3-[(3,4-dichlorobenzyl) sulfanyl]-5-propyl [1,2,4]triazolo[4,3- a]pyrimidin-7(8H)-one |
| 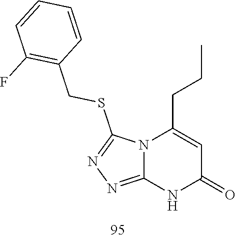 [SMILES: CCCc1cc(=O)[nH]c2nnc(SCc3ccccc3F)n12] | 3-[(2-fluorobenzyl) sulfanyl]-5-propyl [1,2,4]triazolo[4,3-a] pyrimidin- 7(8H)-one |
| 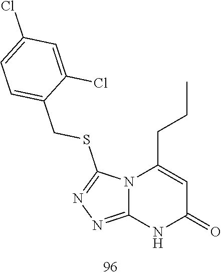 [SMILES: CCCc1cc(=O)[nH]c2nnc(SCc3ccc(Cl)cc3Cl)n12] | 3-[(2,4-dichlorobenzyl) sulfanyl]-5-propyl [1,2,4]triazolo[4,3- a]pyrimidin-7(8H)- one |
| 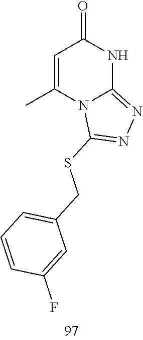 [SMILES: Cc1cc(=O)[nH]c2nnc(SCc3cccc(F)c3)n12] | 3-[(3-fluorobenzyl) sulfanyl]-5-methyl[1,2,4] triazolo[4,3-a]pyrimidin- 7(8H)-one |
| 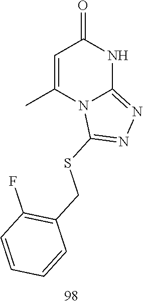 [SMILES: Cc1cc(=O)[nH]c2nnc(SCc3ccccc3F)n12] | 3-[(2-fluorobenzyl) sulfanyl]-5-methyl [1,2,4]triazolo [4,3-a]pyrimidin- 7(8H)-one |
| 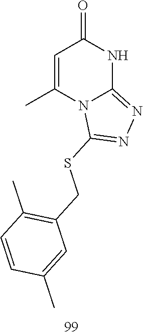 [SMILES: Cc1ccc(C)c(CSc2nnc3[nH]c(=O)cc(C)n23)c1] | 3-[(2,5-dimethylbenzyl) sulfanyl]-5-methyl [1,2,4]triazolo[4,3-a] pyrimidin-7(8H)-one |
| 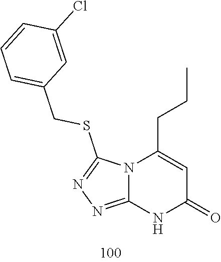 [SMILES: CCCc1cc(=O)[nH]c2nnc(SCc3cccc(Cl)c3)n12] | 3-[(3-chlorobenzyl) sulfanyl]-5-propyl [1,2,4]triazolo [4,3-a]pyrimidin- 7(8H)-one |

### LCMS Methods

### LC-MS Method 1:

Column-YMC-Triat C₁₈ (33×2.1 mm, 3 mm), (mobile phase: 98% [0.05% HCOOH in water] and 2% [CH₃CN] held for 0.75 min, then to 90% [0.05% HCOOH in water] and 10% [CH₃CN] in 1.0 min, further to 2% [0.05% HCOOH in water] and 98% [CH₃CN] in 2.0 min, held this mobile phase composition up to 2.5 min and finally back to initial condition in 3.0 min). Flow=1.0 ml/min.

### LC-MS Method 2:

LCMS/MS-API 2000/Q trap

### Monitoring Method

API 2000 Mass Spectrometer from Applied Biosystems (Single quadrupole mass spectrometer) Ionisation method: Electrospray; Polarity: Positive ions.

Capillary (kV) 5.5, DP (V) 50.00, Entrance Potential (V) 10, Focusing Potential (V) 400, Source Temperature 200° C., Ion Source Gas1 (Psi) 40, Ion Source Gas 2 (Psi) 50, Curtain Gas (Psi) 40 Mass range: 100 to 800 AMU; UV Wavelength range: 220 to 260 nm; Method (Shimadzu Prominance system) with the following HPLC gradient conditions (Solvent A: 10 Mm NH4OAc in Water and Solvent B: Acetonitrile). Flow rate: 1.2 ml/min.

| TIME | MODULE | % A (Buffer) | % B (CH₃CN) |
|---|---|---|---|
| 0.01 | Pumps | 90 | 10 |
| 1.50 | Pumps | 70 | 30 |
| 3.00 | Pumps | 10 | 90 |
| 4.00 | Pumps | 10 | 90 |
| 5.00 | Pumps | 90 | 10 |
| 5.10 | System Controller | Stop | |

Type of column: Zorbax Extend C₁₈; Column length: 50 mm; Internal diameter of column: 4.6 mm; Particle Size: 5 mm

Mass conditions: Ionization technique: ESI (Electron Spray Ionization) using API (Atmospheric pressure Ionization) source; Declustering Potential: 10-70 V depending on the ionization of compound. Mass range: 100-800 amu; Scan type: Q1; Polarity: +ve; Ion Source: Turbo spray; Ion spray voltage: +5500; Mass Source temperature: 200° C.

### LC-MS Method 3:

LCMS/MS-API 2000/Q trap-Monitoring Method

API 2000 Mass Spectrometer from Applied Biosystems (Single quadrupole mass spectrometer) Ionization method: Electrospray; Polarity: Positive ions. Capillary (kV) 5.5, DP (V) 50.00, Entrance Potential (V) 10, Focusing Potential (V) 400, Source Temperature 200° C., Ion Source Gas1 (Psi) 40, Ion Source Gas 2 (Psi) 50, Curtain Gas (Psi) 40; Mass range: 100 to 800 amu; UV Wavelength range: 220 to 260 nm; Method Shimadzu Prominence with the following HPLC gradient conditions (Solvent A: 10 Mm NH4OAc in Water and Solvent B: Acetonitrile). Flow rate: 1.2 ml/min.

| TIME | MODULE | % A (Buffer) | % B (CH₃CN) |
|---|---|---|---|
| 0.01 | Pumps | 90 | 10 |
| 1.50 | Pumps | 70 | 30 |
| 3.00 | Pumps | 10 | 90 |
| 4.00 | Pumps | 10 | 90 |
| 5.00 | Pumps | 90 | 10 |
| 5.10 | System Controller | Stop | |

Type of column: Xbridge C₁₈; Column length: 50 mm; Internal diameter of column: 4.6 mm; Particle Size: 5 micron.

Mass conditions: Ionization technique: ESI (Electron Spray Ionization) using API (Atmospheric pressure Ionization) source; Declustering Potential: 10-70 V depending on the ionization of compound. Mass range: 100-800 amu; Scan type: Q1; Polarity: +ve; Ion Source: Turbo spray; Ion spray voltage: +5500; Mass Source temperature: 200° C.

### LC-MS Method 4: UPLC-MS Method:

Column: XBRIDGE C₁₈; Column length: 50 mm; Internal diameter of column: 4.6 mm; Particle Size: 5 mm.

Gradient condition: Flow rate: 1.5 ml/min; Column Temperature: 50° C.; Injection Volume: 0.5 μL; Waters ACQUITY UPLC with the following HPLC gradient conditions (Solvent A: 5 mM NH₄OAc in Water and Solvent B: 5 mM NH₄OAc in Acetonitrile:Water-90:10).

| TIME | MODULE | % A | % B |
|---|---|---|---|
| 0.01 | Pumps | 98 | 2 |
| 0.75 | Pumps | 98 | 2 |
| 1.25 | Pumps | 85 | 15 |
| 2.50 | Pumps | 30 | 70 |
| 3.75 | Pumps | 2 | 98 |
| 4.50 | Pumps | 2 | 98 |
| 5.00 | Pumps | 98 | 2 |
| 5.10 | Pumps | 98 | 2 |

### Mass Conditions:

ACQUITY SQD Mass Spectrometer from Waters (Single quadruple mass spectrometer, Waters ACQUITY SQD2); Ionization method: Electro spray; Polarity: positive ions; Capillary (kV) 3.50, Cone (V) 40.00, Source Temperature 150° C., Desolvation Temperature 450° C., Cone Gas Flow (L/Hr) 50, Desolvation Gas Flow (L/Hr) 750; Mass range: 100 to 800 Da; DAD (Diode Array Detection); Wavelength range (nm): 210 to 400 nm; HPLC: Waters AQUITY UPLC.

LC-MS method 5: Agilent UPLC-MS:

Column-YMC Triart C₁₈ (2.1×33 mm, 3 mm)

Gradient condition: Flow rate: 1.2 ml/min; Column Temperature: 50° C.; Injection Volume: 0.4 μL Solvent A: 0.01% HCOOH in water and Solvent B: 0.01% HCOOH in CH₃CN, (mobile phase: 95% [0.01% HCOOH in water] and 5% [0.01% HCOOH in CH₃CN] held for 0.50 min then to 1% [0.01% HCOOH in water] and 99% [0.01% HCOOH in CH₃CN] in 3.0 min, held this composition up to 4.00 min and finally back to initial condition in 4.10 min, held for 4.50 min).

| TIME | MODULE | % A | % B |
|---|---|---|---|
| 0.00 | Pumps | 95 | 5 |
| 0.5 | Pumps | 95 | 5 |
| 3.00 | Pumps | 1 | 99 |
| 4.00 | Pumps | 1 | 99 |
| 4.10 | Pumps | 95 | 5 |
| 4.50 | Pumps | 95 | 5 |

Mass conditions: SQD Mass Spectrometer from Agilent (Single quadrupole mass spectrometer); Ionisation method: Electro spray; Polarity: Negative ions Capillary (kV) 4.00, Fragmentor (V) 150.00, Threshold-200, Drying gas Temperature 350° C., Drying Gas Flow (L/min) 12, Nebulizer pressure (Psi) 50.

### Example 2: Inhibitory Activity Against MPC

The inhibition of mitochondrial pyruvate carrier (MPC) by compounds of the invention have been tested as follows and their inhibitory activity was expressed as IC₅₀ₛ values.

### Reagents

Seahorse Xfe24 Flux Analyser [Agilent]

| | |
|---|---|
| Assay Medium: | PBS/CaCl₂/MgCl₂ (Eurobio |
| | CS1PBS00-01) containing 1 mM Pyruvate. |

Following working reagents made up in Assay
Medium at 10x final concentration:

| | |
|---|---|
| Oligomycin A | Stock: 10 mM in DMSO. |
| | Working solution: 20 μM (4 μl/2 ml) |
| fCCP (Carbonyl cyanide 4- | Stock: 10 mM in DMSO |
| (trifluoromethoxy)phenyl- | Working solution: 10 μM (2 μl/2 ml) |
| hydrazone | |
| Rotenone | 10 mM in DMSO |
| | Working solution: 10 μM |
| Antimycin | Stock: 10 mM in EtOH |
| | Working solution: 10 uM |
| | (The rotenone + antimycin |
| | reagents are pooled: |
| | 2 μl rotenone + 2 μl antimycin |
| | in 2 ml Assay Medium) |

### The Day Before

Cells are plated in the customized Seahorse plates. For HeLa cells, seeding is at 4×10⁴ cells/well in 500 ml DMEM, 10% FBS, 1% pen/strep/glutamine. 3 wells are left blank (medium alone) for internal calibration. The assay cartridge is hydrated overnight at 37° C. in 1 ml/well XF Calibrant Solution.

### Assay Day

### Reagent Plate

### Samples/Dilutions Prepared in Assay Medium.

Since 50 μl of compound of the invention will be injected into 450 μl washed cells, compounds are made up in assay medium at 10× the final desired concentration

For dose response analyses, compounds are tested at final concentrations 30, 5, 0.83, 0.14, 0.023 μM (1:6 serial dilutions from 30 mM)

Positive control: 30 mM (the parent hit compound which gave rise to the actual series)

Negative control: Assay medium alone.

The seahorse reagent plate is prepared as follows and transfered to the Xfe24 analyser for the calibration step:

| | |
|---|---|
| Port A | Compound (50 μl) |
| Port B | Oligomycin A (55 μl) |
| Port C | fCCP [(Carbonyl cyanide 4- |
| | (trifluoromethoxy)phenylhydrazone] |
| | (62 μl) |
| Port D | Rotenone + Antimycin (68 μl). |

### Cell Plate

The medium is almost completely removed from the well without allowing cells to dry Cells are washed twice with 300 μl Assay Medium. 450 μl fresh Assay Medium is added to each well. The plate is transferred to the Xfe24 analyser for measurement of pyruvate-dependent OCR. All values are normalized to the initial basal respiration rate in each well which is measured prior to addition of compounds and designated as 100%. IC₅₀ determination is based on the average of the 3 measurements made in the presence of fCCP, taking 30 μM parent hit compound as maximal inhibition and PBS/Ca/Mg as zero inhibition.

Table 2 below presents the IC50s of the compounds of the invention.

TABLE 2

| Compound no | Bret IC₅₀ (nM) | SeaHorse IC₅₀ (nM) |
|---|---|---|
| 3 | 2199 | |
| 7 | 10200 | |
| 8 | 5393 | |
| 9 | 878 | 185 |
| 12 | 561 | 158 |
| 14 | 1752 | |
| 15 | 2090 | |
| 17 | 2030 | |
| 18 | 4210 | |
| 19 | 3484 | |
| 25 | 593 | 171 |
| 29 | 5812 | |
| 30 | 943 | 1646 |
| 31 | 5659 | |
| 33 | 1319 | 1044 |
| 35 | 5925 | |
| 39 | 593 | |
| 40 | 391 | 218 |
| 41 | 129 | |
| 43 | 814 | 289 |
| 45 | 117 | 64 |
| 46 | 4686 | |
| 47 | 167 | 184 |
| 48 | 873 | |
| 50 | 11177 | |
| 51 | 3647 | |
| 85 | 12500 | |
| 90 | 2500 | |

### Example 3: Effet of MPC Inhibition During In Vitro Priming of CD8 T Cells on Memory Marker Expression

OT1 splenocytes were cultured for 3 days at a concentration of 106 cells per mL in RPMI medium (Gibco 61870-01) supplemented with 10% FBS, (Gibco 10270-106), 1% Penicillin/Streptomycin (Gibco 15070-063), 50 μM ß-mercaptoethanol, 1% HEPES (Gibco 15630-080), 1× Non-essential amino acids (Gibco 11140-035), 1% L-glutamine (Gibco 25030-081), 1 mM Sodium Pyruvate (Gibco 11360-039). Cells were additionally supplemented with hIL-2 100 U/ml (Glaxo-IMB), ovalbumin N4 peptide (257-264) 1 μg/ml (model peptide antigen from ovalbumin) and either different concentrations of Compound 45 or 47 or with their solvent DMSO as a control. At day 3, splenocytes were collected, washed and split, and cells were cultured for 4 additional days with 100 U/ml hIL-2 and hIL-7 (Peprotech 200-07) supplemented either with Compound 45 or 47 or DMSO. At day 7, flow cytometry analyses were performed for surface marker expression.

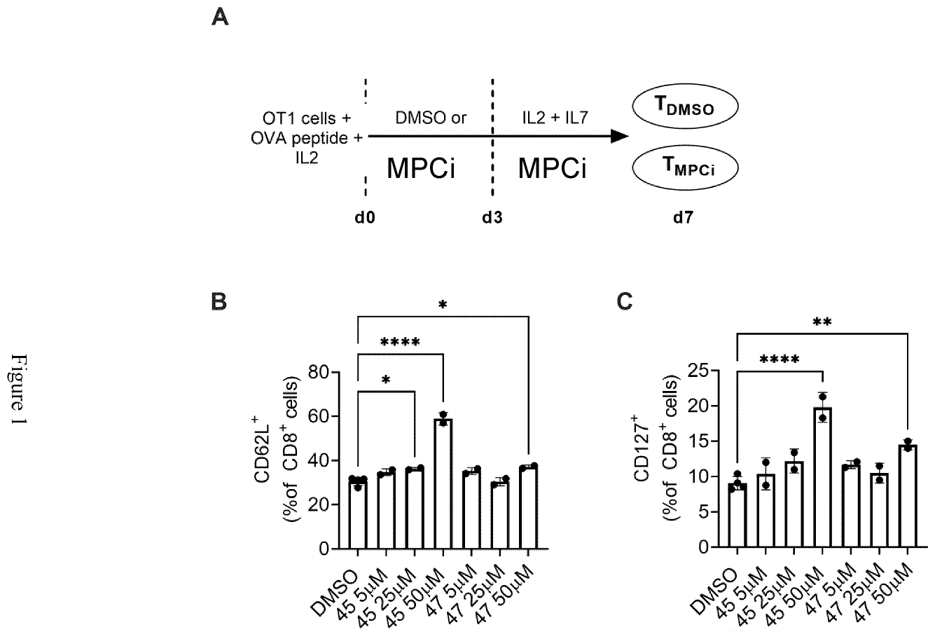

To evaluate the effects of MPC inhibition on T cell differentiation, OTI splenocytes were cultured in the presence of 1 μg/ml ovalbumin-derived N4 peptide, 100 IU/ml recombinant human IL-2 (rhIL-2) and the MPC inhibitors Compound 45 or 47 or control DMSO for 3 days. The cells were cultured for 4 more days in the presence of 100 IU/ml IL-2 and 10 ng/ml rhIL-7 (FIG. 1A) and the inhibitors or DMSO. When analyzing the surface expression of the central memory marker CD62L (FIG. 1B) and the pro-survival receptor CD127 (FIG. 1C) by flow cytometry on day 7, an increase in cells treated with the highest doses of compounds of the invention was observed compared to DMSO.

Therefore, those data support that treating CD8 T cells with a compound of the invention results in enhanced memory characteristics.

### Example 4: Adoptive Cell Transfer Therapy CD8 T Cells Treated with a Compound of the Invention

The effects of the compounds of the invention are tested in an adoptive cell transfer therapy model with CD8 T cells treated as follows and in a melanoma tumor model.

### Adoptive Cell Transfer

Activated CD45.1+OT-1 splenocytes were culture in vitro for 7 days as described above, collected and purified on a Ficoll gradient, allowing to separate dead and live splenocytes. Live splenocytes were counted with Trypan blue stain 0.4%. 100′000 or 2′000′000 live splenocytes were transferred into CD45.2+ host mice by tail vein injection.

To assess the functional capacity of the generated memory T cells, the Compound 45-treated cells were further tested in a mouse model of melanoma.

### Melanoma Tumor Model

B16-OVA cells were cultured in DMEM (GIBCO) with 10% FBS and 1% P/S before their subcutaneous injection into the mouse flank. Each mouse received 100′000 cells in a volume of 200 μl of PBS. 6 days after B16-OVA cells injection, tumors were measured, mice were randomized and lymphodepleted by irradiation (5 Gray). 7 days after B16-Ova injection, mice were adoptively transferred with the ACT protocol described previously. Following the ACT, mice received a vaccination of CpG (50 μg/mouse) and N4 Ova peptide (10 μg/mouse) diluted in PBS to obtain a total volume of 100 μl/mouse, injected subcutaneously at the tail base. Tumors were measured every 2 days and the tumor volume was calculated according to the formula: V=π×[d2×D]/6, where d is the minor tumor axis and D is the major tumor axis. At day 26 days post-tumor engraftment, tumors and spleens were dissected were then stained for flow cytometry analyses.

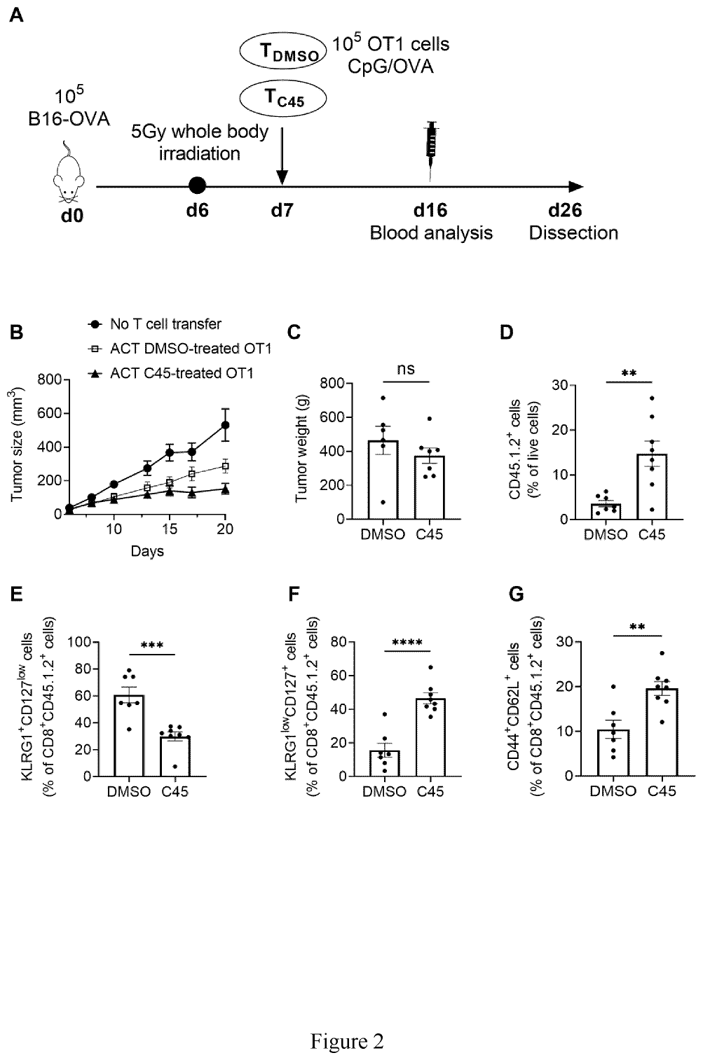

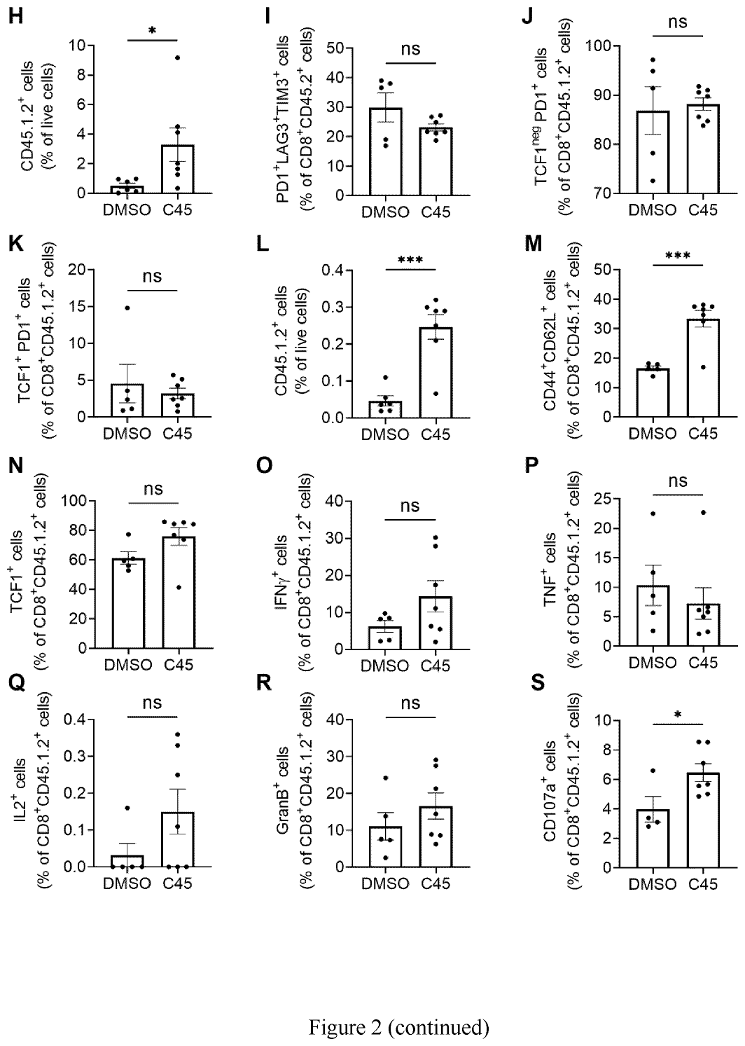

Briefly, 105 ovalbumin-expressing B16 melanoma cells were injected subcutaneously in 6-week-old mice. At day 6 post-engraftment, when a palpable tumor was present, mice were irradiated with 5Gy. The next day 105 Compound 45- or DMSO-treated OT1 cells were intravenously injected, followed by a subcutaneous vaccination of 50 μg CpG and 10 μg N4 Ova peptide (FIG. 2A). It has been shown before that memory CD8+ T cells are more potent in controlling tumor growth, and indeed, B16 tumor growth was reduced in mice that received OT1 CD8+ T cells pre-treated with Compound 45 as compared to DMSO (FIG. 2B). Tumor weight was however not significantly reduced due to high variability (FIG. 2C). When analysing the blood 9 days post-adoptive cell transfer, one could observe an increase in number of transferred cells when treated in vitro with Compound 45 as compared to DMSO (FIG. 2D). The phenotype of the Compound 45-treated cells was biased towards memory, as there was a reduced percentage of short-lived effector T cells (FIG. 2E) and an increase in memory-precursor effector T cells and cells with a central memory phenotype (FIGS. 2F and 2G). When analysing the tumor upon dissection, more Compound 45-treated T cells were infiltrating the tumor (FIG. 2H). Tumor infiltrating Compound 45-treated T cells were however not significantly more or less exhausted as compared to DMSO-treated T cells (FIG. 2I). There was neither a difference in terminally exhausted or progenitor exhausted T cells (FIGS. 2J and 2K). In the spleen, the number of Compound 45-treated T cells was also increased (FIG. 2L) and they displayed an increased central memory phenotype (FIG. 2M), while TCF1 expression was not significantly altered (FIG. 2N). When restimulating the single cell population of the tumor in vitro with the OVA peptide, OT1 T cells treated with Compound 45 did not show a significant increase in IFNy, TNF, IL2 or Granzyme B expression (FIGS. 2O-R), but had increased expression of CD107a (LAMP1) on the cells surface membrane, indicating increased degranulation, which has been correlated with improved cytotoxic potential (FIG. 2S).

Those data supports that CD8 T cells treated with a compound of the invention show enhanced anti-tumoral activity.

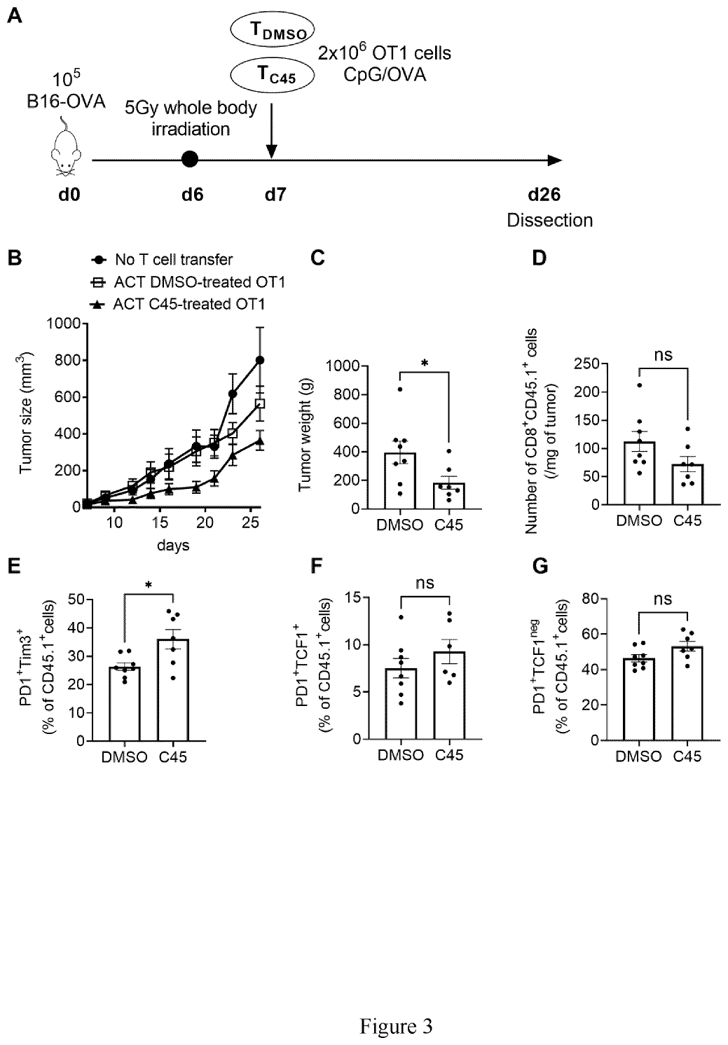

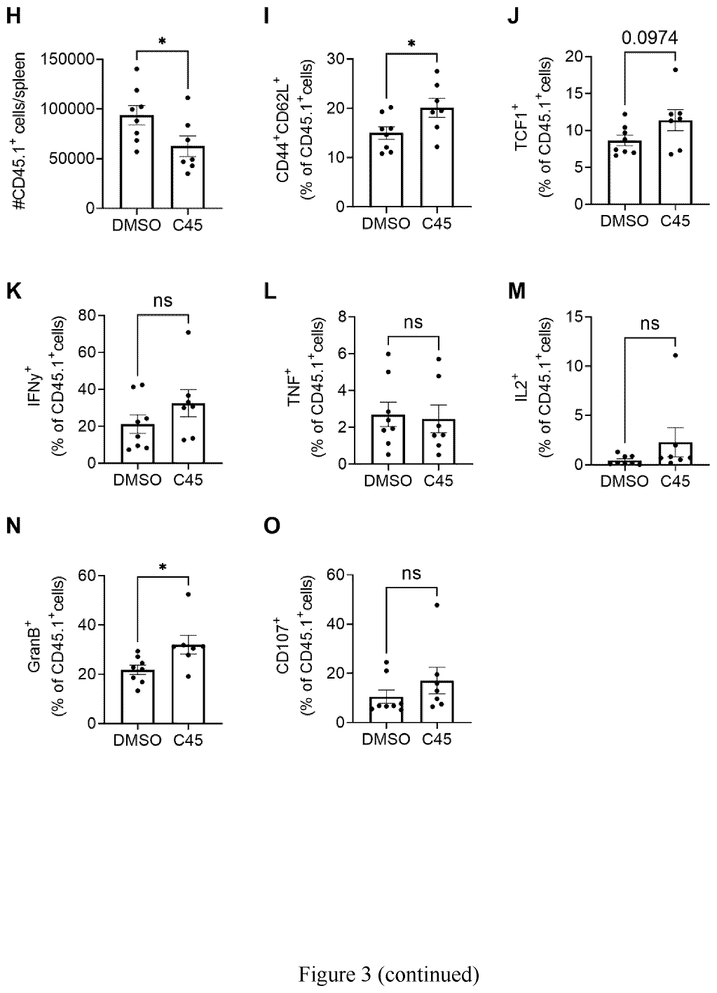

In order to observe a stronger anti-tumor response of the transferred OT1 T cells, the previous experiment was repeated by transferring 2×10⁶ DMSO- or Compound 45-treated OT1 T cells, instead of only 10⁵ (FIG. 3A). With those settings, B16 tumor growth and weight was strongly reduced in mice that received OT1 CD8 T cells pre-treated with Compound 45 as compared to DMSO (FIGS. 3B and 3C). When analysing the tumor upon dissection, no difference in T cell infiltration was observed (FIG. 3D). Surprisingly, tumor infiltrating Compound 45-treated T cells were more exhausted as compared to DMSO-treated T cells (FIG. 3E). There was however no difference in terminally exhausted or progenitor exhausted T cells (FIGS. 3F and 3G). In the spleen, the number of Compound 45-treated T cells was decreased (FIG. 3H), but they displayed an increased central memory phenotype (FIG. 3I), and a trend towards increased TCF1 expression (FIG. 3J). When restimulating the single cell population of the tumor in vitro with the OVA peptide, OT1 T cells treated with Compound 45 did not show difference in IFNy, TNF or IL2 expression (FIGS. 3K-3M), but showed increased Granzyme B expression (FIG. 3N). There was no difference in CD107a expression (FIG. 3O).

Those data support that Adoptive cell transfer therapy CD8 T cells treated with a compound of the invention is better able to control tumor growth.

### Example 5: Effects of MPC Inhibition During the Production of Murine CAR T Cells Upon Adoptive Cell Transfer Therapy

The above data have been obtained in mice using CD8 T cells isolated from transgenic OT1 mice, which are designed to express one unique T cell receptor recognising a peptide sequence of the chicken ovalbumin protein (N4 peptide) when presented on MHC class I molecules.

It was further investigated if compound of the invention could be useful when applied during the production of mouse CAR T cells by usding a CAR construct recognising human HER2, an oncogene frequently involved in human breast cancer, containing a 4-1BB costimulatory domain.

Retrovirus preparation (protocol modified from Tschumi et al., 2018, J Immunother Cancer.; 6 (1): 71)

For each retroviral preparation, 8×10⁶ Phoenix ECO cells (ATCC, CRL-3214) were plated in a T150 tissue culture flask in RPMI medium supplemented with 10% FCS, 10 mM HEPES and 50 U/ml Penicillin-Streptomycin. On the next day, cells were transfected with 21 μg of the retroviral construct with Turbofect transfection reagent (Thermo Fischer Scientific), according to the manufacturer protocol. The medium was changed daily and collected at 48 h and 72 h post transfection. 48 h and 72 h virus supernatants were pooled and sedimented at 22′000 rcf for 2 h at 4° C. Finally, retrovirus pellets were resuspended in 2 ml of full RPMI medium and divided in 8 aliquots of 250 μl each, which were snap-frozen on dry ice and stored at −80° C.

T Cell Transduction (Protocol Modified from Tschumi et al., 2018, Supra)

Spleens from wild type CD45.1.2 mice were smashed on a 70 μm cell strainer. CD8 T cells were purified using the Easy SepTM Mouse CD8+ T Cell Isolation Kit (StemCell) according to the manufacturer protocol. 0.5×106 CD8 T cells were plated in 48 well plates in 0.5 ml of complete RPMI 1640 medium supplemented with 10% FCS, antibiotics and 50 IU/ml of recombinant human IL-2, and exposed to either DMSO or 20 μM Compound 45. Mouse T-cells were activated with Activator CD3/CD28 Dynabeads (Gibco) at a ratio of 2 beads per cell. Retroviral infection was conducted at 37° C. for 24 h. Untreated 48 well plates were coated for 24 h with 20 μg/ml of recombinant human fibronectin (Takara Clontech) at 4° C., followed by PBS 2% BSA for 30 min at RT and finally washed with PBS. One aliquot of concentrated retroviruses was plated in each fibronectin-coated 48 well plates and centrifuged for 90 min at 2′000 rcf and 32° C. Then, 0.5×10⁶ of 24 h-activated CD8 T cells were added on top of the viruses and spun for 10 min at 400 rcf and 32° C. On day 3, the medium was replaced with 10 IU/ml recombinant human IL-2, 10 ng/ml recombinant human IL-7 and 10 ng/ml recombinant human IL-15, containing either DMSO or 20 μM Compound 45. Cells were then split every second day.

Adoptive Cell Transfer (Protocol Modified from Tschumi et al., 2018, Supra)

CD45.2 C57BL/6 mice were engrafted subcutaneously with 4×10⁵ B16F10 tumors modified to express HER2. Six days later, mice were lymphodepleted with 100 mg/kg cyclophosphamide (Sigma Aldrich, C7397) injected i.p., and homogeneous groups were constituted with regard to tumor volume. T cells (5×10⁶) were adoptively transferred i.v. on the next day. Tumor volumes were measured three times a week with a caliper and calculated using the formula: V=π×[d2×D]/6, where d is the minor tumor axis and D is the major tumor axis. Tumors were collected and separated from skin. Single cell suspensions were obtained with the Mouse Tumor Dissociation Kit (Miltenyi, 130-096-730) according to the manufacturer protocol. Spleen and draining lymph node were smashed on a 70 μm cell strainer. Single cell suspensions were stained with antibodies before flow cytometry analysis.

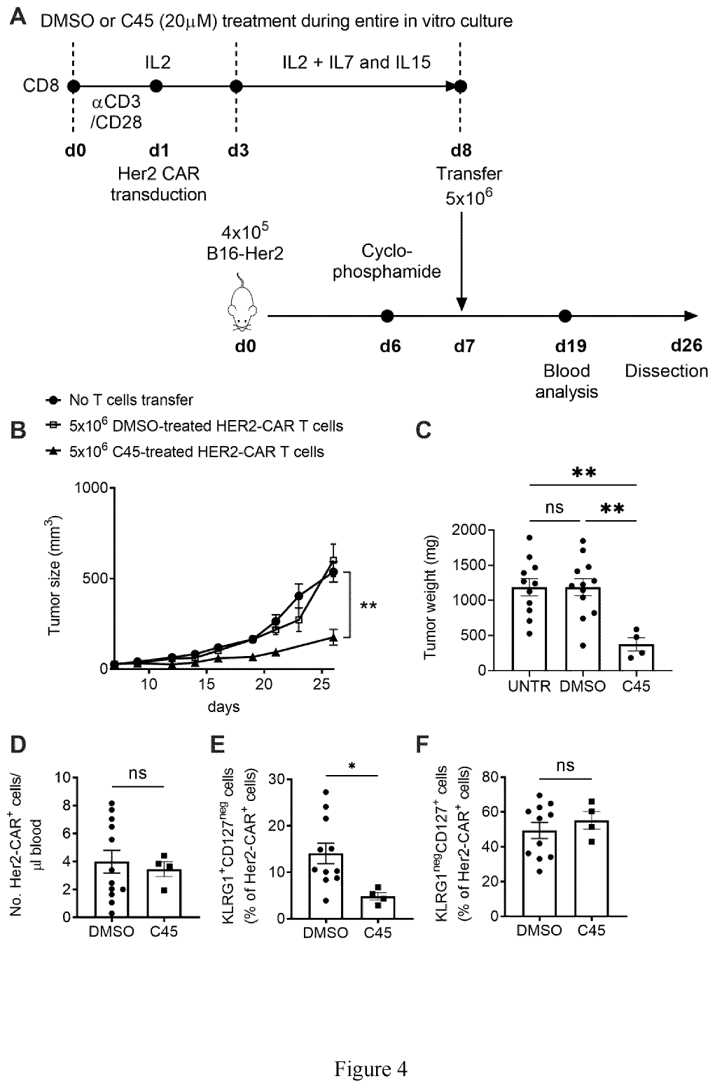

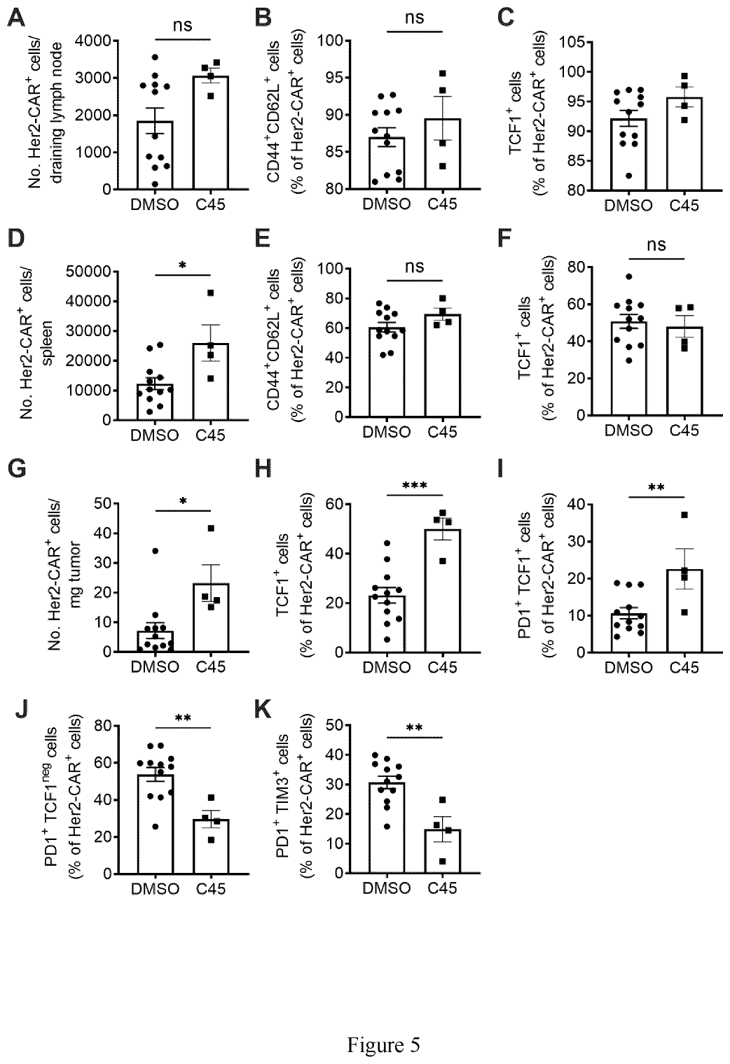

Polyclonal CD8 T cells from wild type mice were activated in the presence of DMSO or 20 μM Compound 45 and then retrovirally transduced with a HER2-CAR construct (FIG. 4A). When adoptively transferring (ACT) the T cells in mice bearing B16 melanoma tumors expressing HER2, only Compound 45-treated HER2CAR T cell ACT was able to significantly suppress tumor growth (FIGS. 4B and 4C). When analysing the blood 12 days after ACT, no difference in the number of CAR T cells was observed (FIG. 4D), however, the percentage of Compound 45-HER2CAR T cells forming short-lived effector T cells was significantly reduced (FIG. 4E). No difference in percentages of memory precursor effector T cells could be detected (FIG. 4F) and TCF1-expressing T cells (FIG. 4G). A trend towards an increased engraftment of Compound 45-HER2CAR T cells was observed in the tumor-draining lymph nodes, while this increase was significant in the spleen (FIGS. 5A and 5D). Neither in the draining lymph node nor in the spleen could be observed a difference in central memory T cell differentiation (FIGS. 5B and 5E) or in HER2CAR T cells expressing the memory-specific transcription factor TCF1 (FIGS. 5C and 5F). Tumors of Compound 45-HER2CAR-treated mice contained significantly more CAR T cells (FIG. 5G). More tumor-infiltrating Compound 45-treated CAR T cells expressed TCF1 (FIG. 5H). Interestingly, when looking at the co-expression of TCF1 with PD1, Compound 45-treated CAR T cells formed more progenitor exhausted T cells (TCF1-positive) and less terminally differentiated exhausted T cells (TCF1-negative) (FIGS. 51 and 5J), indicative of an increased stem cell-like phenotype which might benefit from combination therapy with checkpoint blockade immunotherapy. Finally, fewer Compound 45-treated CAR T cells were expressing the inhibitory molecules PD1 and TIM3, indicating a less exhausted phenotype (FIG. 5K).

Altogether, those data support that the compounds of the invention are useful during the production of CAR T cells by improving their memory phenotype and antitumor function upon adoptive cell transfer therapy.

1-19. (canceled)

20. A method for treating a subject suffering from a disease or disorder, wherein said disease or disorder is selected from a cancer, an auto-immune disease such as multiple sclerosis, a metabolic diseases such as type 2 diabetes, an hair loss disorder such as alopecia, a neurodegenerative disorder such as Parkinson or Alzheimer's disease, a fibrotic disease such as pulmonary fibrosis or non alcoholic steatohepatitis (NASH), a skin or tissue injury such a skin wound or a burn, and an acute pathology of the brain such as stroke or brain trauma, said method comprising administering an effective amount of one or more compound of Formula (I) in a subject in need thereof 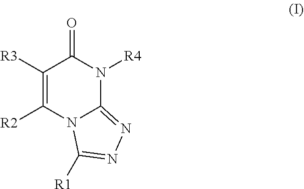
wherein R1 is a moiety R5-R6; R2 is selected from H, optionally substituted C₁-C₆ alkyl, optionally substituted aryl, optionally substituted heteroaryl, optionally substituted aryl C₁-C₆ alkyl, optionally substituted heteroaryl C₁-C₆ alkyl, optionally substituted C₁-C₆ alkoxy such as optionally substituted C₁-C₆ alkoxy substituted C₁-C₆ alkyl, optionally substituted C₃-C₈ heterocycloalkyl and optionally substituted C₃-C₈ cycloalkyl such as optionally substituted cyclopropyl; R3 is selected from H and optionally substituted C₁-C₆ alkyl and wherein at least one of R2 and R3 is not H; R4 is H; R5 is selected from a bond, S, SO₂, NR9 and O; R6 is a group —(CR10R11)ₙ—R7 wherein n is an integer between 0 and 2; R7 is selected from optionally substituted C₁-C₆ alkyl, optionally substituted C₂-C₆ alkenyl, optionally substituted heterocycle, optionally substituted aryl such as optionally substituted phenyl, optionally substituted heteroaryl, cyano and —C(O)—R8; R8 is selected from optionally substituted amino, optionally substituted alkoxy and optionally substituted aryl; R9 is H or optionally substituted C₁-C₆; R10 and R11 are independently selected from is H and optionally substituted C₁-C₆ alkyl; as well as tautomers, geometrical isomers, optically active forms and pharmaceutically acceptable salts and pharmaceutically active derivative thereof.

21. The method according to claim 20, wherein R1 is a moiety S—R6.

22. The method according to claim 20, wherein R1 is a moiety NR9-R6.

23. The method according to claim 20, wherein R1 is a moiety R6.

24. The method according to claim 20, wherein R2 is optionally substituted C₁-C₆ alkyl.

25. The method according to claim 20, wherein R3 is optionally substituted C₁-C₆ alkyl.

26. The method according to claim 20, wherein R3 is H.

27. The method according to claim 20, wherein R4 is H.

28. The method for use according to claim 20, wherein R7 is optionally substituted C₁-C₆ alkyl.

29. The method according to claim 20, wherein R7 is optionally substituted C₂-C₆ alkenyl.

30. The method according to claim 20, wherein R7 is an optionally substituted aryl.

31. The method according to claim 20, wherein n is an integer selected from 0 and 1, R5 is S and R7 is an optionally substituted aryl.

32. The method according to claim 20, wherein R7 is an optionally substituted heteroaryl.

33. The method according to claim 20, wherein said compounds are selected from the following group:
3-[(3,4-difluorobenzyl)sulfanyl]-5-propyl[1,2,4]triazolo[4,3-a]pyrimidin-7 (8H)-one;
3-[(2-chloro-5-fluorobenzyl)sulfanyl]-5-propyl[1,2,4]triazolo[4,3-a]pyrimidin-7 (8H)-one;
3-[(3,5-difluorobenzyl)sulfanyl]-5-propyl[1,2,4]triazolo[4,3-a]pyrimidin-7 (8H)-one;
3-{[3-(difluoromethyl)benzyl]sulfanyl}-5-propyl[1,2,4]triazolo[4,3-a]pyrimidin-7 (8H)-one;
3-{[2-(difluoromethyl)benzyl]sulfanyl}-5-propyl[1,2,4]triazolo[4,3-a]pyrimidin-7 (8H)-one;
3-{[4-(difluoromethyl)benzyl]sulfanyl}-5-propyl[1,2,4]triazolo[4,3-a]pyrimidin-7 (8H)-one;
3-{[(7-oxo-5-propyl-7,8-dihydro[1,2,4]triazolo[4,3-a]pyrimidin-3-yl)sulfanyl]methyl}benzonitrile;
4-fluoro-2-{[(7-oxo-5-propyl-7,8-dihydro[1,2,4]triazolo[4,3-a]pyrimidin-3-yl)sulfanyl]methyl}benzonitrile;
3-[(5-fluoro-2-methoxybenzyl)sulfanyl]-5-propyl[1,2,4]triazolo[4,3-a]pyrimidin-7 (8H)-one;
3-[(3-chloro-5-fluorobenzyl)sulfanyl]-5-propyl[1,2,4]triazolo[4,3-a]pyrimidin-7 (8H)-one;
3-[(4-chloro-3-fluorobenzyl)sulfanyl]-5-propyl[1,2,4]triazolo[4,3-a]pyrimidin-7 (8H)-one;
3-[(5-fluoro-2-methylbenzyl)sulfanyl]-5-propyl[1,2,4]triazolo[4,3-a]pyrimidin-7 (8H)-one);
3-[(4-fluoro-2-methylbenzyl)sulfanyl]-5-propyl[1,2,4]triazolo[4,3-a]pyrimidin-7 (8H)-one1;
3-[(2-phenylethyl)sulfanyl]-5-propyl[1,2,4]triazolo[4,3-a]pyrimidin-7 (8H)-one;
3-[(furan-3-ylmethyl)sulfanyl]-5-propyl[1,2,4]triazolo[4,3-a]pyrimidin-7 (8H)-one;
3-[(2-methoxybenzyl)sulfanyl]-5-propyl[1,2,4]triazolo[4,3-a]pyrimidin-7 (8H)-one;
3-[(3-methoxybenzyl)sulfanyl]-5-propyl[1,2,4]triazolo[4,3-a]pyrimidin-7 (8H)-one;
4-{[(7-oxo-5-propyl-7,8-dihydro[1,2,4]triazolo[4,3-a]pyrimidin-3-yl)sulfanyl]methyl}benzonitrile;
3-[(1-benzofuran-5-ylmethyl)sulfanyl]-5-propyl[1,2,4]triazolo[4,3-a]pyrimidin-7 (8H)-one;
3-[(4-methoxybenzyl)sulfanyl]-5-propyl[1,2,4]triazolo[4,3-a]pyrimidin-7 (8H)-one;
3-[(2,5-difluorobenzyl)sulfanyl]-5-propyl[1,2,4]triazolo[4,3-a]pyrimidin-7 (8H)-one;
3-[(2,4-difluorobenzyl)sulfanyl]-5-propyl[1,2,4]triazolo[4,3-a]pyrimidin-7 (8H)-one;
3-[(2,6-difluorobenzyl)sulfanyl]-5-propyl[1,2,4]triazolo[4,3-a]pyrimidin-7 (8H)-one;
5-propyl-3-[(1H-pyrazol-4-ylmethyl)sulfanyl][1,2,4]triazolo[4,3-a]pyrimidin-7 (8H)-one;
3-[(2,6-dimethylbenzyl)sulfanyl]-5-propyl[1,2,4]triazolo[4,3-a]pyrimidin-7 (8H)-one;
3-[(2-chloro-6-fluorobenzyl)sulfanyl]-5-propyl[1,2,4]triazolo[4,3-a]pyrimidin-7 (8H)-one;
3-[(2,5-dimethylbenzyl)sulfanyl]-5-propyl[1,2,4]triazolo[4,3-a]pyrimidin-7 (8H)-one;
3-{[2-(2,4-difluorophenyl)ethyl]sulfanyl}-5-propyl[1,2,4]triazolo[4,3-a]pyrimidin-7 (8H)-one;
3-[(2,5-difluorobenzyl)sulfanyl]-5-methyl[1,2,4]triazolo[4,3-a]pyrimidin-7 (8H)-one;
5-[2-(benzyloxy)ethyl]-3-[(2,5-difluorobenzyl)sulfanyl][1,2,4]triazolo[4,3-a]pyrimidin-7 (8H)-one;
3-[(2,5-difluorobenzyl)sulfanyl]-5-(methoxymethyl) [1,2,4]triazolo[4,3-a]pyrimidin-7 (8H)-one;
5-{2-[(4-chlorobenzyl)oxy] ethyl}-3-[(2,5-difluorobenzyl)sulfanyl][1,2,4]triazolo[4,3-a]pyrimidin-7 (8H)-one;
5-{2-[(2-chlorobenzyl)oxy]ethyl}-3-[(2,5-difluorobenzyl)sulfanyl][1,2,4]triazolo[4,3-a]pyrimidin-7 (8H)-one;
5-{2-[(3-chlorobenzyl)oxy]ethyl}-3-[(2,5-difluorobenzyl)sulfanyl][1,2,4]triazolo[4,3-a]pyrimidin-7 (8H)-one;
3-[(2,5-difluorobenzyl)sulfanyl]-5-(2-methoxyethyl) [1,2,4]triazolo[4,3-a]pyrimidin-7 (8H)-one;
3-[(2-chloro-6-fluorobenzyl)sulfanyl]-5-methyl[1,2,4]triazolo[4,3-a]pyrimidin-7 (8H)-one;
3-[(2-chloro-6-fluorobenzyl)sulfanyl]-5-ethyl[1,2,4]triazolo[4,3-a]pyrimidin-7 (8H)-one;
5-butyl-3-[(2-chloro-6-fluorobenzyl)sulfanyl][1,2,4]triazolo[4,3-a]pyrimidin-7 (8H)-one;
3-[(2-chloro-6-fluorobenzyl)sulfanyl]-5-cyclopropyl[1,2,4]triazolo[4,3-a]pyrimidin-7 (8H)-one;
3-[(2-chloro-6-fluorobenzyl)sulfanyl]-5-(2-methylpropyl) [1,2,4]triazolo[4,3-a]pyrimidin-7 (8H)-one;
3-[(2-chloro-6-fluorobenzyl)sulfanyl]-5-(cyclopropylmethyl) [1,2,4]triazolo[4,3-a]pyrimidin-7 (8H)-one;
3-[(2-chloro-6-fluorobenzyl)sulfanyl]-5-(methoxymethyl) [1,2,4]triazolo[4,3-a]pyrimidin-7 (8H)-one;
5-[2-(benzyloxy)ethyl]-3-[(2-chloro-6-fluorobenzyl)sulfanyl][1,2,4]triazolo[4,3-a]pyrimidin-7 (1H)-one;
3-[(2-chloro-6-fluorobenzyl)sulfanyl]-5-(3,3,3-trifluoropropyl) [1,2,4]triazolo[4,3-a]pyrimidin-7 (8H)-one;
5-benzyl-3-[(2-chloro-6-fluorobenzyl)sulfanyl][1,2,4]triazolo[4,3-a]pyrimidin-7 (8H)-one;
4-{[(5-benzyl-7-oxo-7,8-dihydro[1,2,4]triazolo[4,3-a]pyrimidin-3-yl)sulfanyl]methyl}benzonitrile;
3-(phenylsulfanyl)-5-propyl[1,2,4]triazolo[4,3-a]pyrimidin-7 (8H)-one;
3-[(2-chloro-6-fluorobenzyl)sulfanyl]-6-methyl-5-propyl[1,2,4]triazolo[4,3-a]pyrimidin-7 (8H)-one;
3-[(2-chloro-6-fluorobenzyl)amino]-5-propyl[1,2,4]triazolo[4,3-a]pyrimidin-7 (8H)-one;
3-[(2-chloro-6-fluorobenzyl)sulfonyl]-5-propyl[1,2,4]triazolo[4,3-a]pyrimidin-7 (8H)-one;
3-(2-phenylethyl)-5-propyl[1,2,4]triazolo[4,3-a]pyrimidin-7 (8H)-one;
3-[(2-chlorobenzyl)sulfanyl]-5-methyl[1,2,4]triazolo[4,3-a]pyrimidin-7 (8H)-one;
5-methyl-3-(methylsulfanyl) [1,2,4]triazolo[4,3-a]pyrimidin-7 (8H)-one;
5-methyl-3-[(4-nitrobenzyl)sulfanyl][1,2,4]triazolo[4,3-a]pyrimidin-7 (8H)-one;
3-{[(2E)-3-phenylprop-2-en-1-yl]sulfanyl}-5-propyl[1,2,4]triazolo[4,3-a]pyrimidin-7 (8H)-one;
N-(4-ethoxyphenyl)-2-[(7-oxo-5-propyl-7,8-dihydro[1,2,4]triazolo[4,3-a]pyrimidin-3-yl) sulfanyl]acetamide;
methyl5-{[(7-oxo-5-propyl-7,8-dihydro[1,2,4]triazolo[4,3-a]pyrimidin-3-yl)sulfanyl]methyl}furan-2-carboxylate;
ethyl2-[(7-oxo-5-propyl-7,8-dihydro[1,2,4]triazolo[4,3-a]pyrimidin-3-yl)sulfanyl]propanoate;
ethyl2-[(7-oxo-5-propyl-7,8-dihydro[1,2,4]triazolo[4,3-a]pyrimidin-3-yl)sulfanyl]butanoate;
methyl2-[(7-oxo-5-propyl-7,8-dihydro[1,2,4]triazolo[4,3-a]pyrimidin-3-yl)sulfanyl]butanoate;
methyl2-[(7-oxo-5-propyl-7,8-dihydro[1,2,4]triazolo[4,3-a]pyrimidin-3-yl)sulfanyl]propanoate;
benzyl[(7-oxo-5-propyl-7,8-dihydro[1,2,4]triazolo[4,3-a]pyrimidin-3-yl)sulfanyl]acetate;
[(7-oxo-5-propyl-7,8-dihydro[1,2,4]triazolo[4,3-a]pyrimidin-3-yl)sulfanyl]acetonitrile;
3-[(2-oxo-2-phenylethyl)sulfanyl]-5-propyl[1,2,4]triazolo[4,3-a]pyrimidin-7 (8H)-one;
3-{[2-(4-methoxyphenyl)-2-oxoethyl]sulfanyl}-5-propyl[1,2,4]triazolo[4,3-a]pyrimidin-7 (8H)-one;
N-(5-chloro-2-methoxyphenyl)-2-[(7-oxo-5-propyl-7,8-dihydro[1,2,4]triazolo[4,3-a]pyrimidin-3-yl)sulfanyl]acetamide;
5,6-dimethyl-3-(propylsulfanyl) [1,2,4]triazolo[4,3-a]pyrimidin-7 (8H)-one;
5,6-dimethyl-3-[(3-methylbutyl)sulfanyl][1,2,4]triazolo[4,3-a]pyrimidin-7 (8H)-one;
5,6-dimethyl-3-[(4-nitrobenzyl)sulfanyl][1,2,4]triazolo[4,3-a]pyrimidin-7 (8H)-one;
5,6-dimethyl-3-[(3-methylbenzyl)sulfanyl][1,2,4]triazolo[4,3-a]pyrimidin-7 (8H)-one;
3-[(2,5-dimethylbenzyl)sulfanyl]-5,6-dimethyl[1,2,4]triazolo[4,3-a]pyrimidin-7 (8H)-one;
5,6-dimethyl-3-[(4-methylbenzyl)sulfanyl][1,2,4]triazolo[4,3-a]pyrimidin-7 (8H)-one;
5,6-dimethyl-3-[(3-nitrobenzyl)sulfanyl][1,2,4]triazolo[4,3-a]pyrimidin-7 (8H)-one;
3-[(4-chlorobenzyl)sulfanyl]-5,6-dimethyl[1,2,4]triazolo[4,3-a]pyrimidin-7 (8H)-one;
3-[(2-chlorobenzyl)sulfanyl]-5,6-dimethyl[1,2,4]triazolo[4,3-a]pyrimidin-7 (8H)-one;
5,6-dimethyl-3-{[3-(trifluoromethyl)benzyl]sulfanyl}[1,2,4]triazolo[4,3-a]pyrimidin-7 (8H)-one;
5,6-dimethyl-3-{[4-(trifluoromethyl)benzyl]sulfanyl}[1,2,4]triazolo[4,3-a]pyrimidin-7 (8H)-one;
3-[(2-fluorobenzyl)sulfanyl]-5,6-dimethyl[1,2,4]triazolo[4,3-a]pyrimidin-7 (8H)-one;
3-[(3-chlorobenzyl)sulfanyl]-5,6-dimethyl[1,2,4]triazolo[4,3-a]pyrimidin-7 (8H)-one;
3-[(2,4-dichlorobenzyl)sulfanyl]-5,6-dimethyl[1,2,4]triazolo[4,3-a]pyrimidin-7 (8H)-one;
3-[(3-chlorobenzyl)sulfanyl]-5-methyl[1,2,4]triazolo[4,3-a]pyrimidin-7 (8H)-one;
3-[(3-fluorobenzyl)sulfanyl]-5,6-dimethyl[1,2,4]triazolo[4,3-a]pyrimidin-7 (8H)-one;
3-[(2-chloro-6-fluorobenzyl)sulfanyl]-5,6-dimethyl[1,2,4]triazolo[4,3-a]pyrimidin-7 (8H)-one;
3-[(4-bromobenzyl)sulfanyl]-5,6-dimethyl[1,2,4]triazolo[4,3-a]pyrimidin-7 (8H)-one;
3-[(4-chlorobenzyl)sulfanyl]-5-methyl[1,2,4]triazolo[4,3-a]pyrimidin-7 (8H)-one;
3-(benzylsulfanyl)-5-propyl[1,2,4]triazolo[4,3-a]pyrimidin-7 (8H)-one;
3-[(2-methylbenzyl)sulfanyl]-5-propyl[1,2,4]triazolo[4,3-a]pyrimidin-7 (8H)-one;
3-[(3-methylbenzyl)sulfanyl]-5-propyl[1,2,4]triazolo[4,3-a]pyrimidin-7 (8H)-one;
3-[(4-methylbenzyl)sulfanyl]-5-propyl[1,2,4]triazolo[4,3-a]pyrimidin-7 (8H)-one;
3-[(naphthalen-1-ylmethyl)sulfanyl]-5-propyl[1,2,4]triazolo[4,3-a]pyrimidin-7 (8H)-one;
3-[(3-fluorobenzyl)sulfanyl]-5-propyl[1,2,4]triazolo[4,3-a]pyrimidin-7 (8H)-one;
3-[(4-fluorobenzyl)sulfanyl]-5-propyl[1,2,4]triazolo[4,3-a]pyrimidin-7 (8H)-one;
3-[(2-chlorobenzyl)sulfanyl]-5-propyl[1,2,4]triazolo[4,3-a]pyrimidin-7 (8H)-one;
3-[(3,4-dichlorobenzyl)sulfanyl]-5-propyl[1,2,4]triazolo[4,3-a]pyrimidin-7 (8H)-one;
3-[(2-fluorobenzyl)sulfanyl]-5-propyl[1,2,4]triazolo[4,3-a]pyrimidin-7 (8H)-one;
3-[(2,4-dichlorobenzyl)sulfanyl]-5-propyl[1,2,4]triazolo[4,3-a]pyrimidin-7 (8H)-one;
3-[(3-fluorobenzyl)sulfanyl]-5-methyl[1,2,4]triazolo[4,3-a]pyrimidin-7 (8H)-one;
3-[(2-fluorobenzyl)sulfanyl]-5-methyl[1,2,4]triazolo[4,3-a]pyrimidin-7 (8H)-one;
3-[(2,5-dimethylbenzyl)sulfanyl]-5-methyl[1,2,4]triazolo[4,3-a]pyrimidin-7 (8H)-one; and
3-[(3-chlorobenzyl)sulfanyl]-5-propyl[1,2,4]triazolo[4,3-a]pyrimidin-7 (8H)-one.

34. A compound selected from the following group:
3-[(3,4-difluorobenzyl)sulfanyl]-5-propyl[1,2,4]triazolo[4,3-a]pyrimidin-7 (8H)-one;
3-[(2-chloro-5-fluorobenzyl)sulfanyl]-5-propyl[1,2,4]triazolo[4,3-a]pyrimidin-7 (8H)-one;
3-[(3,5-difluorobenzyl)sulfanyl]-5-propyl[1,2,4]triazolo[4,3-a]pyrimidin-7 (8H)-one;
3-{[3-(difluoromethyl)benzyl]sulfanyl}-5-propyl[1,2,4]triazolo[4,3-a]pyrimidin-7 (8H)-one;
3-{[2-(difluoromethyl)benzyl]sulfanyl}-5-propyl[1,2,4]triazolo[4,3-a]pyrimidin-7 (8H)-one;
3-{[4-(difluoromethyl)benzyl]sulfanyl}-5-propyl[1,2,4]triazolo[4,3-a]pyrimidin-7 (8H)-one;
3-{[(7-oxo-5-propyl-7,8-dihydro[1,2,4]triazolo[4,3-a]pyrimidin-3-yl)sulfanyl]methyl}benzonitrile;
4-fluoro-2-{[(7-oxo-5-propyl-7,8-dihydro[1,2,4]triazolo[4,3-a]pyrimidin-3-yl)sulfanyl]methyl}benzonitrile;
3-[(5-fluoro-2-methoxybenzyl)sulfanyl]-5-propyl[1,2,4]triazolo[4,3-a]pyrimidin-7 (8H)-one;
3-[(3-chloro-5-fluorobenzyl)sulfanyl]-5-propyl[1,2,4]triazolo[4,3-a]pyrimidin-7 (8H)-one;
3-[(4-chloro-3-fluorobenzyl)sulfanyl]-5-propyl[1,2,4]triazolo[4,3-a]pyrimidin-7 (8H)-one;
3-[(5-fluoro-2-methylbenzyl)sulfanyl]-5-propyl[1,2,4]triazolo[4,3-a]pyrimidin-7 (8H)-one;
3-[(4-fluoro-2-methylbenzyl)sulfanyl]-5-propyl[1,2,4]triazolo[4,3-a]pyrimidin-7 (8H)-one;
3-[(2-phenylethyl)sulfanyl]-5-propyl[1,2,4]triazolo[4,3-a]pyrimidin-7 (8H)-one;
3-[(furan-3-ylmethyl)sulfanyl]-5-propyl[1,2,4]triazolo[4,3-a]pyrimidin-7 (8H)-one;
3-[(2-methoxybenzyl)sulfanyl]-5-propyl[1,2,4]triazolo[4,3-a]pyrimidin-7 (8H)-one;
3-[(3-methoxybenzyl)sulfanyl]-5-propyl[1,2,4]triazolo[4,3-a]pyrimidin-7 (8H)-one;
4-{[(7-oxo-5-propyl-7,8-dihydro[1,2,4]triazolo[4,3-a]pyrimidin-3-yl)sulfanyl]methyl}benzonitrile;
3-[(1-benzofuran-5-ylmethyl)sulfanyl]-5-propyl[1,2,4]triazolo[4,3-a]pyrimidin-7 (8H)-one;
3-[(4-methoxybenzyl)sulfanyl]-5-propyl[1,2,4]triazolo[4,3-a]pyrimidin-7 (8H)-one;
3-[(2,5-difluorobenzyl)sulfanyl]-5-propyl[1,2,4]triazolo[4,3-a]pyrimidin-7 (8H)-one;
3-[(2,4-difluorobenzyl)sulfanyl]-5-propyl[1,2,4]triazolo[4,3-a]pyrimidin-7 (8H)-one;
3-[(2,6-difluorobenzyl)sulfanyl]-5-propyl[1,2,4]triazolo[4,3-a]pyrimidin-7 (8H)-one;
5-propyl-3-[(1H-pyrazol-4-ylmethyl)sulfanyl][1,2,4]triazolo[4,3-a]pyrimidin-7 (8H)-one;
3-[(2,6-dimethylbenzyl)sulfanyl]-5-propyl[1,2,4]triazolo[4,3-a]pyrimidin-7 (8H)-one;
3-[(2-chloro-6-fluorobenzyl)sulfanyl]-5-propyl[1,2,4]triazolo[4,3-a]pyrimidin-7 (8H)-one;
3-[(2,5-dimethylbenzyl)sulfanyl]-5-propyl[1,2,4]triazolo[4,3-a]pyrimidin-7 (8H)-one;
3-{[2-(2,4-difluorophenyl)ethyl]sulfanyl}-5-propyl[1,2,4]triazolo[4,3-a]pyrimidin-7 (8H)-one;
3-[(2,5-difluorobenzyl)sulfanyl]-5-methyl[1,2,4]triazolo[4,3-a]pyrimidin-7 (8H)-one;
5-[2-(benzyloxy)ethyl]-3-[(2,5-difluorobenzyl)sulfanyl][1,2,4]triazolo[4,3-a]pyrimidin-7 (8H)-one;
3-[(2,5-difluorobenzyl)sulfanyl]-5-(methoxymethyl) [1,2,4]triazolo[4,3-a]pyrimidin-7 (8H)-one;
5-{2-[(4-chlorobenzyl)oxy]ethyl}-3-[(2,5-difluorobenzyl)sulfanyl][1,2,4]triazolo[4,3-a]pyrimidin-7 (8H)-one;
5-{2-[(2-chlorobenzyl)oxy]ethyl}-3-[(2,5-difluorobenzyl)sulfanyl][1,2,4]triazolo[4,3-a]pyrimidin-7 (8H)-one;
5-{2-[(3-chlorobenzyl)oxy]ethyl}-3-[(2,5-difluorobenzyl)sulfanyl][1,2,4]triazolo[4,3-a]pyrimidin-7 (8H)-one;
3-[(2,5-difluorobenzyl)sulfanyl]-5-(2-methoxyethyl) [1,2,4]triazolo[4,3-a]pyrimidin-7 (8H)-one;
3-[(2-chloro-6-fluorobenzyl)sulfanyl]-5-methyl[1,2,4]triazolo[4,3-a]pyrimidin-7 (8H)-one;
3-[(2-chloro-6-fluorobenzyl)sulfanyl]-5-ethyl[1,2,4]triazolo[4,3-a]pyrimidin-7 (8H)-one;
5-butyl-3-[(2-chloro-6-fluorobenzyl)sulfanyl][1,2,4]triazolo[4,3-a]pyrimidin-7 (8H)-one;
3-[(2-chloro-6-fluorobenzyl)sulfanyl]-5-cyclopropyl[1,2,4]triazolo[4,3-a]pyrimidin-7 (8H)-one;
3-[(2-chloro-6-fluorobenzyl)sulfanyl]-5-(2-methylpropyl) [1,2,4]triazolo[4,3-a]pyrimidin-7 (8H)-one;
3-[(2-chloro-6-fluorobenzyl)sulfanyl]-5-(cyclopropylmethyl) [1,2,4]triazolo[4,3-a]pyrimidin-7 (8H)-one;
3-[(2-chloro-6-fluorobenzyl)sulfanyl]-5-(methoxymethyl) [1,2,4]triazolo[4,3-a]pyrimidin-7 (8H)-one;
5-[2-(benzyloxy)ethyl]-3-[(2-chloro-6-fluorobenzyl)sulfanyl][1,2,4]triazolo[4,3-a]pyrimidin-7 (1H)-one;
3-[(2-chloro-6-fluorobenzyl)sulfanyl]-5-(3,3,3-trifluoropropyl) [1,2,4]triazolo[4,3-a]pyrimidin-7 (8H)-one;
5-benzyl-3-[(2-chloro-6-fluorobenzyl)sulfanyl][1,2,4]triazolo[4,3-a]pyrimidin-7 (8H)-one;
4-{[(5-benzyl-7-oxo-7,8-dihydro[1,2,4]triazolo[4,3-a]pyrimidin-3-yl)sulfanyl]methyl}benzonitrile;
3-(phenylsulfanyl)-5-propyl[1,2,4]triazolo[4,3-a]pyrimidin-7 (8H)-one;
3-[(2-chloro-6-fluorobenzyl)sulfanyl]-6-methyl-5-propyl[1,2,4]triazolo[4,3-a]pyrimidin-7 (8H)-one;
3-[(2-chloro-6-fluorobenzyl)amino]-5-propyl[1,2,4]triazolo[4,3-a]pyrimidin-7 (8H)-one;
3-[(2-chloro-6-fluorobenzyl)sulfonyl]-5-propyl[1,2,4]triazolo[4,3-a]pyrimidin-7 (8H)-one; and
3-(2-phenylethyl)-5-propyl[1,2,4]triazolo[4,3-a]pyrimidin-7 (8H)-one.

35. A pharmaceutical composition containing at least one compound as described in claim 20, as well as tautomers, geometrical isomers, optically active forms and pharmaceutically acceptable salts thereof combined with at least one anti-cancer immunotherapeutic agent such as CAR T-cells or an immune checkpoint inhibitor and at least one pharmaceutically acceptable carrier, diluent or excipient thereof.

36. A pharmaceutical composition containing at least one compound according to claim 34, and at least one pharmaceutically acceptable carrier, diluent or excipient thereof.

37. An in vitro method for obtaining and/or maintaining T-cells with a memory phenotype, said method comprising the following steps:
providing at least one T-cell that has the ability to differentiate into a memory phenotype, such as CD8+ or CD4+ T cell;
contacting said at least one T-cell with at least one compound as described in claim 20 or a mixture thereof;
culturing the cells in a T-cell culture medium;
isolating the obtained T-cells.

38. An in vitro method for obtaining and/or maintaining T-cells with a memory phenotype, said method comprising the following steps:
providing at least one T-cell that has the ability to differentiate into a memory phenotype, such as CD8+ or CD4+ T cell;
contacting said at least one T-cell with at least one compound as described in claim 34 or a mixture thereof;
culturing the cells in a T-cell culture medium;
isolating the obtained T-cells.
